(19) United States
(12) Patent Application Publication
(10) Pub. No.: US 2003/0124679 A1
(43) Pub. Date: Jul. 3, 2003

(54) Glycosylated kinamycins and methods of making and using them

(76) Inventors: Jay M. Short, Rancho Santa Fe, CA (US); Ashish Paradkar, San Diego, CA (US); Mustafa Varoglu, San Diego, CA (US); Eric J. Mathur, Carlsbad, CA (US)

Correspondence Address: GREGORY P. EINHORN, Fish & Richardson P.C., Suite 500, 4350 La Jolla Village Drive, San Diego, CA, 92122, US

(21) Appl. No.: 10/187,267

(22) Filed: Jun. 27, 2002

Related U.S. Application Data
(60) Provisional application No. 60/301,401, filed on Jun. 27, 2001.

(51) Int. Cl. C07H 17/08; C12P 19/62; C12N 9/10; C07H 21/04; C12Q 1/68; C12P 21/02; C12N 1/21; C12N 15/74

(52) U.S. Cl. 435/76; 435/69.1; 435/320.1; 435/252.3; 536/7.1; 536/23.2; 435/193; 514/28; 435/6

(57) ABSTRACT

The invention provides compositions, including pharmaceuticals, comprising glycosylated kinamycins. The compositions and methods of the invention can be used to treat infections, i.e., as antibiotics, and as anti-tumor agents. The compositions of the invention can also act as act as electrophilic azo-coupling agents in vitro or in vivo. The invention also provides enzymes capable of generating kinamycin, nucleic acids that encode them, antibodies that bind to them, and methods for making and using them.

### TECHNICAL FIELD

This invention generally pertains to the fields of medicine and bacteriology. Specifically, the compositions of the invention comprise glycosylated kinamycins. The compositions and methods of the invention can be used to treat infections, i.e., as antibiotics, and as anti-tumor agents. The compositions of the invention can also act as act as electrophilic azo-coupling agents in vitro or in vivo. The invention also provides enzymes capable of generating kinamycin, nucleic acids that encode them, antibodies that bind to them, and methods for making and using them.

### BACKGROUND

Kinamycins, a class of type II polyketides, are used to treat infections, as described, e.g., in “Structures and biological properties of Kinamycin A, B, C, and D,” Chem Pharm Bull (Tokyo), 1973 May;21(5):931-40; and, “A new antibiotic, kinamycin: fermentation, isolation, purification and properties,” J. Antibiot. (Tokyo). 1971 June, 24(6):353-9. The genes for most of the biosynthesis of kinamycin from Streptomyces murayamaensis have been cloned and heterologously expressed, see, e.g., Gould, et al., J. Antibiot. (Tokyo) 1998 January;51(1):50-7. It has been speculated that kinamycins owe their anti-tumor and antibiotic properties to their ability to act as electrophilic azo-coupling agents in vivo, see, e.g., Laufer, et al., J. Am. Chem. Soc. Mar. 6, 2002;124(9):1854-5.

### SUMMARY

The invention provides glycosylated kinamycins and polyketides comprising glycosylated kinamycins. The compositions and methods of the invention can be used to treat infections, i.e., as antibiotics, and as anti-tumor agents. The compositions of the invention can also act as act as electrophilic azo-coupling agents in vitro or in vivo.

These compositions can be made using in vivo systems (e.g., in bacteria or using bacterial extracts, or equivalents) and then isolated, or, they can be partly or entirely made by synthetic procedures. In one aspect, the isolated or synthetically made glycosylated polyketide comprises least one saccharide moiety. The saccharide can comprise one or more polysaccharides. In one aspect, the saccharide comprises a 2, 6 dideoxysugar, such as a digitose, e.g., an L-digitose. The saccharide can also comprise an olivose, a lactose, a galactose, a glucose or a fructose. The polyketide can comprise a type II polyketide, such as a kinamycin. The kinamycin can comprise an aglycone kinamycin. The invention also provides isolated kinamycin molecules, wherein the kinamycin molecule comprises at least one saccharide moiety.

The invention provides pharmaceutical compositions comprising a polyketide, wherein the polyketide comprises a kinamycin comprising at least one saccharide moiety, and a pharmaceutically acceptable carrier. The invention provides pharmaceutical compositions comprising a kinamycin, wherein the kinamycin comprises at least one saccharide moiety, and a pharmaceutically acceptable carrier. In the pharmaceutical compositions of the invention, any pharmaceutically acceptable carrier can be used, e.g., the pharmaceutically acceptable carrier and/or the pharmaceutical compositions can be solids or liquids. In one aspect of the pharmaceutical compositions, the saccharide comprises one or more polysaccharides. The saccharide can comprise a 2, 6 dideoxysugar, such as a digitose. The digitose can comprise an L-digitose. The saccharides can comprise an olivose, a lactose, a galactose, a glucose or a fructose. The polyketide can comprise a type II polyketide. The kinamycin can be an aglycone kinamycin.

The invention provides polyketides comprising a glycosylated kinamycin made by a process comprising the following steps: (a) providing a nucleic acid comprising a Streptococcus murayamaensis nucleic acid sequence comprising an insert deposited as ATCC accession no. ______; (b) providing (i) a Streptococcus sp. or a Dactylosporangium sp. bacillus, or (ii) an intracellular extract of a Streptococcus sp. or a Dactylosporangium sp.; and (c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated polyketide comprising a glycosylated kinamycin. The process can further comprise isolating the glycosylated kinamycin. The polyketides can comprise a type II polyketide. The glycosylation can comprise a saccharide. The saccharide can further comprise a polysaccharide. The saccharide can comprise a 2, 6 dideoxysugar, such as a digitose, e.g., an L-digitose. The saccharide can comprise an olivose, a lactose, a galactose, a glucose or a fructose. The kinamycin can be an aglycone kinamycin. In the process, the Streptococcus sp. of step (b) can be a S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus, a S. violaceoruber, or a S. diversa. The Dactylosporangium sp. of step (b) can be a Dactylosporangium sp. ATCC 53693.

The invention provides an isolated glycosylated kinamycin made by a process comprising the following steps: (a) providing a nucleic acid comprising a Streptococcus murayamaensis nucleic acid sequence comprising an insert deposited as ATCC accession no. ______; (b) providing (i) a Streptococcus sp. bacillus, or (ii) an intracellular extract of a Streptococcus sp.; and (c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated kinamycin. In the process, the Streptococcus sp. of step (b) can be a S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus, a S. violaceoruber, or a S. diversa. The.Dactylosporangium sp. of step (b) can be a Dactylosporangium sp. ATCC 53693.

The invention provides a method for making a composition comprising a glycosylated kinamycin comprising the following steps: (a) providing a nucleic acid comprising a Streptococcus murayamaensis nucleic acid sequence comprising an insert deposited as ATCC accession no. ______; (b) providing (i) a Streptococcus sp. or a Dactylosporangium sp. bacillus, or (ii) an intracellular extract of a Streptococcus sp. or a Dactylosporangium sp.; (c) inserting the nucleic acid into the Streptococcus sp. of step (b) or contacting the nucleic acid with the intracellular extract of step (b) under conditions wherein the nucleic acid is transcribed into transcription products and the transcription products are translated into polypeptides, thereby making a glycosylated kinamycin. In one aspect, the method further comprises isolating the glycosylated kinamycin. The polyketide can comprise a type II polyketide. The glycosylation can comprise a saccharide. The saccharide can further comprise a polysaccharide. The saccharide can comprise a 2, 6 dideoxysugar, such as a digitose, e.g., an L-digitose. The saccharide can comprise an olivose, a lactose, a galactose, a glucose or a fructose. The kinamycin can be an aglycone kinamycin. The Streptococcus sp. of step (b) can be a S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus or a S. violaceoruber, or extracts thereof, or equivalents thereof. The Dactylosporangium sp. of step (b) can be a Dactylosporangium sp. ATCC 53693, or extracts thereof, or equivalents thereof.

The invention provides an isolated composition comprising a compound having a general formula as set forth as DS2 in FIG. 3. The invention provides an isolated composition comprising a compound having a general formula as set forth as DS1a in FIG. 1. These compositions can further comprise a saccharide, such as a 2, 6 dideoxysugar, e.g., a digitose, such as an L-digitose. The saccharide also can comprise an olivose, a lactose, a galactose, a glucose or a fructose.

The invention provides an isolated composition comprising a compound having a general formula as set forth as DS1 in FIG. 1. The invention also provides compounds having a general formula as set forth as DS1 with different or more saccharide moieties, such as a 2, 6 dideoxysugar, e.g., a digitose, such as an L-digitose. The alternative saccharides also can comprise an olivose, a lactose, a galactose, a glucose or a fructose.

The invention provides an isolated or recombinant nucleic acid comprising a nucleic acid sequence having 95% sequence identity to SEQ ID NO:1, wherein the nucleic acid, when expressed in a Streptococcus, results in the synthesis of a kinamycin molecule in the Streptococcus. The invention provides an isolated or recombinant nucleic acid comprising a nucleic acid sequence as set forth in SEQ ID NO:1, wherein the nucleic acid, when expressed in a Streptococcus, results in the synthesis of a kinamycin molecule in the Streptococcus.

The invention provides an isolated or recombinant nucleic acid comprising a nucleic acid sequence (a) having a sequence identity to SEQ ID NO:2, wherein the sequence identity is at least 85%, 90%, 95%, or 98%, and the sequence identities are determined by analysis with a sequence comparison algorithm or by a visual inspection; (b) having a sequence as set forth in SEQ ID NO:2; (c) that hybridizes under stringent conditions to a sequence comprising SEQ ID NO:2; or (d) encoding a polypeptide as set forth in SEQ ID NO: 3. The invention provides an isolated or recombinant polypeptide comprising a sequence (a) having a sequence identity to SEQ ID NO:3, wherein the sequence identity is at least 85%, 90%, 95%, or 98%, and the sequence identities are determined by analysis with a sequence comparison algorithm or by a visual inspection; (b) having a sequence as set forth in SEQ ID NO:3; or (c) encoded by a nucleic acid of the invention as set forth above. In one aspect, the polypeptide functions an enzyme of the kinamycin biosynthetic pathway, in particular, this polypeptide can be gene 1 of the exemplary kinamycin biosynthetic pathway as set forth in FIG. 2. In one aspect, the polypeptide is 594 residues in length.

The invention provides an isolated or recombinant nucleic acid comprising a nucleic acid sequence (a) having a sequence identity to SEQ ID NO:4, wherein the sequence identity is at least 70%, 75%, 80%, 85%, 90%, 95%, or 98%, and the sequence identities are determined by analysis with a sequence comparison algorithm or by a visual inspection; (b) having a sequence as set forth in SEQ ID NO:4; (c) that hybridizes under stringent conditions to a sequence comprising SEQ ID NO:4; or (d) encoding a polypeptide as set forth in SEQ ID NO:5. The invention provides an isolated or recombinant polypeptide comprising a sequence (a) having a sequence identity to SEQ ID NO:5, wherein the sequence identity is at least 70%, 75%, 80%, 85%, 90%, 95%, or 98%, and the sequence identities are determined by analysis with a sequence comparison algorithm or by a visual inspection; (b) having a sequence as set forth in SEQ ID NO:5; or (c) encoded by a nucleic acid of the invention as set forth above. In one aspect, the polypeptide functions an enzyme of the kinamycin biosynthetic pathway, in particular, this polypeptide can be gene 2 of the exemplary kinamycin biosynthetic pathway as set forth in FIG. 2. In one aspect, the polypeptide is 171, residues in length.

The invention provides an isolated or recombinant nucleic acid comprising a nucleic acid sequence (a) having a sequence identity to SEQ ID NO:6, wherein the sequence identity is at least 80%, 85%, 90%, 95%, or 98%, and the sequence identities are determined by analysis with a sequence comparison algorithm or by a visual inspection; (b) having a sequence as set forth in SEQ ID NO:6; (c) that hybridizes under stringent conditions to a sequence comprising SEQ ID NO:6; or (d) encoding a polypeptide as set forth in SEQ ID NO:7. The invention provides an isolated or recombinant polypeptide comprising a sequence (a) having a sequence identity to SEQ ID NO:7, wherein the sequence identity is at least 80%, 85%, 90%, 95%, or 98%, and the sequence identities are determined by analysis with a sequence comparison algorithm or by a visual inspection; (b) having a sequence as set forth in SEQ ID NO:7; or (c) encoded by a nucleic acid of the invention as set forth above. In one aspect, the polypeptide functions an enzyme of the kinamycin biosynthetic pathway, in particular, this polypeptide can be gene 3 of the exemplary kinamycin biosynthetic pathway as set forth in FIG. 2. In one aspect, the polypeptide is 132 residues in length.

The invention provides an isolated or recombinant nucleic acid comprising a nucleic acid sequence (a) having a sequence identity to SEQ ID NO:8, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:8; (c) that hybridizes under stringent conditions to a sequence comprising SEQ ID NO:8; or (d) encoding a polypeptide as set forth in SEQ ID NO:9. The invention provides an isolated or recombinant polypeptide comprising a sequence (a) having a sequence identity to SEQ ID NO:9, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:9; or (c) encoded by a nucleic acid of the invention as set forth above. In one aspect, the polypeptide functions an enzyme of the kinamycin biosynthetic pathway, in particular, this polypeptide can be gene 4 of the exemplary kinamycin biosynthetic pathway as set forth in FIG. 2. In one aspect, the polypeptide is 244 residues in length.

The invention provides an isolated or recombinant nucleic acid comprising a nucleic acid sequence (a) having a sequence identity to SEQ ID NO:10, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:10; (c) that hybridizes under stringent conditions to a sequence comprising SEQ ID NO:10; or (d) encoding a polypeptide as set forth in SEQ ID NO:11. The invention provides an isolated or recombinant polypeptide comprising a sequence (a) having a sequence identity to SEQ ID NO: 11, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:11; or (c) encoded by a nucleic acid of the invention as set forth above. In one aspect, the polypeptide functions an enzyme of the kinamycin biosynthetic pathway, in particular, this polypeptide can be gene 5 of the exemplary kinamycin biosynthetic pathway as set forth in FIG. 2. In one aspect, the polypeptide is 121 residues in length.

The invention provides an isolated or recombinant nucleic acid comprising a nucleic acid sequence (a) having a sequence identity to SEQ ID NO:12, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:12; (c) that hybridizes under stringent conditions to a sequence comprising SEQ ID NO:12; or (d) encoding a polypeptide as set forth in SEQ ID NO:13. The invention provides an isolated or recombinant polypeptide comprising a sequence (a) having a sequence identity to SEQ ID NO: 13, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:13; or (c) encoded by a nucleic acid of the invention as set forth above. In one aspect, the polypeptide functions an enzyme of the kinamycin biosynthetic pathway, in particular, this polypeptide can be gene 6 of the exemplary kinamycin biosynthetic pathway as set forth in FIG. 2. In one aspect, the polypeptide is 982 residues in length.

The invention provides an isolated or recombinant nucleic acid comprising a nucleic acid sequence (a) having a sequence identity to SEQ ID NO:14, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:14; (c) that hybridizes under stringent conditions to a sequence comprising SEQ ID NO:14; or (d) encoding a polypeptide as set forth in SEQ ID NO:15. The invention provides an isolated or recombinant polypeptide comprising a sequence (a) having a sequence identity to SEQ ID NO: 15, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:15; or (c) encoded by a nucleic acid of the invention as set forth above. In one aspect, the polypeptide functions an enzyme of the kinamycin biosynthetic pathway, in particular, this polypeptide can be gene 7 of the exemplary kinamycin biosynthetic pathway as set forth in FIG. 2. In one aspect, the polypeptide is 197 residues in length.

The invention provides an isolated or recombinant nucleic acid comprising a nucleic acid sequence (a) having a sequence identity to SEQ ID NO:16, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:16; (c) that hybridizes under stringent conditions to a sequence comprising SEQ ID NO:16; or (d) encoding a polypeptide as set forth in SEQ ID NO:17. The invention provides an isolated or recombinant polypeptide comprising a sequence (a) having a sequence identity to SEQ ID NO: 17, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:17; or (c) encoded by a nucleic acid of the invention as set forth above. In one aspect, the polypeptide functions an enzyme of the kinamycin biosynthetic pathway, in particular, this polypeptide can be gene 8 of the exemplary kinamycin biosynthetic pathway as set forth in FIG. 2. In one aspect, the polypeptide is 139 residues in length.

The invention provides an isolated or recombinant nucleic acid comprising a nucleic acid sequence (a) having a sequence identity to SEQ ID NO:18, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:18; (c) that hybridizes under stringent conditions to a sequence comprising SEQ ID NO:18; or (d) encoding a polypeptide as set forth in SEQ ID NO:19. The invention provides an isolated or recombinant polypeptide comprising a sequence (a) having a sequence identity to SEQ ID NO: 19, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:19; or (c) encoded by a nucleic acid of the invention as set forth above. In one aspect, the polypeptide functions an enzyme of the kinamycin biosynthetic pathway, in particular, this polypeptide can be gene 9 of the exemplary kinamycin biosynthetic pathway as set forth in FIG. 2. In one aspect, the polypeptide is 148 residues in length.

The invention provides an isolated or recombinant nucleic acid comprising a nucleic acid sequence (a) having a sequence identity to SEQ ID NO:20, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:20; (c) that hybridizes under stringent conditions to a sequence comprising SEQ ID NO:20; or (d) encoding a polypeptide as set forth in SEQ ID NO:21. The invention provides an isolated or recombinant polypeptide comprising a sequence (a) having a sequence identity to SEQ ID NO: 21, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:21; or (c) encoded by a nucleic acid of the invention as set forth above. In one aspect, the polypeptide functions an enzyme of the kinamycin biosynthetic pathway, in particular, this polypeptide can be gene 10 of the exemplary kinamycin biosynthetic pathway as set forth in FIG. 2. In one aspect, the polypeptide is 489 residues in length.

The invention provides an isolated or recombinant nucleic acid comprising a nucleic acid sequence (a) having a sequence identity to SEQ ID NO:22, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:22; (c) that hybridizes under stringent conditions to a sequence comprising SEQ ID NO:22; or (d) encoding a polypeptide as set forth in SEQ ID NO:23. The invention provides an isolated or recombinant polypeptide comprising a sequence (a) having a sequence identity to SEQ ID NO: 23, wherein the sequence identity is at least 65%, 70%, 75%, 80%, 85%, 90%, 95%, or 98%, and the sequence identities are determined by analysis with a sequence comparison algorithm or by a visual inspection; (b) having a sequence as set forth in SEQ ID NO:23; or (c) encoded by a nucleic acid of the invention as set forth above. In one aspect, the polypeptide functions an enzyme of the kinamycin biosynthetic pathway, in particular, this polypeptide can be gene 11 of the exemplary kinamycin biosynthetic pathway as set forth in FIG. 2. In one aspect, the polypeptide is 229 residues in length.

The invention provides an isolated or recombinant nucleic acid comprising a nucleic acid sequence (a) having a sequence identity to SEQ ID NO:24, wherein the sequence identity is at least 95% or 98% and the sequence identities are determined by analysis with a sequence comparison algorithm or by a visual inspection; (b) having a sequence as set forth in SEQ ID NO:24; (c) that hybridizes under stringent conditions to a sequence comprising SEQ ID NO:24; or (d) encoding a polypeptide as set forth in SEQ ID NO: 25. The invention provides an isolated or recombinant polypeptide comprising a sequence (a) having a sequence identity to SEQ ID NO:25, wherein the sequence identity is at least 95% or 98% and the sequence identities are determined by analysis with a sequence comparison algorithm or by a visual inspection; (b) having a sequence as set forth in SEQ ID NO:25; or (c) encoded by a nucleic acid of the invention as set forth above. In one aspect, the polypeptide functions an enzyme of the kinamycin biosynthetic pathway, in particular, this polypeptide can be gene 12 of the exemplary kinamycin biosynthetic pathway as set forth in FIG. 2. In one aspect, the polypeptide is 109 residues in length.

The invention provides an isolated or recombinant nucleic acid comprising a nucleic acid sequence (a) having a sequence identity to SEQ ID NO:26, wherein the sequence identity is at least 90%, 95% or 98% and the sequence identities are determined by analysis with a sequence comparison algorithm or by a visual inspection; (b) having a sequence as set forth in SEQ ID NO:26; (c) that hybridizes under stringent conditions to a sequence comprising SEQ ID NO:26; or (d) encoding a polypeptide as set forth in SEQ ID NO: 27. The invention provides an isolated or recombinant polypeptide comprising a sequence (a) having a sequence identity to SEQ ID NO:27, wherein the sequence identity is at least 90%, 95% or 98% and the sequence identities are determined by analysis with a sequence comparison algorithm or by a visual inspection; (b) having a sequence as set forth in SEQ ID NO:27; or (c) encoded by a nucleic acid of the invention as set forth above. In one aspect, the polypeptide functions an enzyme of the kinamycin biosynthetic pathway, in particular, this polypeptide can be gene 13 of the exemplary kinamycin biosynthetic pathway as set forth in FIG. 2. In one aspect, the polypeptide is 424 residues in length.

The invention provides an isolated or recombinant nucleic acid comprising a nucleic acid sequence (a) having a sequence identity to SEQ ID NO:28, wherein the sequence identity is at least 85%, 90%, 95% or 98% and the sequence identities are determined by analysis with a sequence comparison algorithm or by a visual inspection; (b) having a sequence as set forth in SEQ ID NO:28; (c) that hybridizes under stringent conditions to a sequence comprising SEQ ID NO:28; or (d) encoding a polypeptide as set forth in SEQ ID NO:29. The invention provides an isolated or recombinant polypeptide comprising a sequence (a) having a sequence identity to SEQ ID NO:29, wherein the sequence identity is at least 85%, 90%, 95% or 98% and the sequence identities are determined by analysis with a sequence comparison algorithm or by a visual inspection; (b) having a sequence as set forth in SEQ ID NO:29; or (c) encoded by a nucleic acid of the invention as set forth above. In one aspect, the polypeptide functions an enzyme of the kinamycin biosynthetic pathway, in particular, this polypeptide can be gene 14 of the exemplary kinamycin biosynthetic pathway as set forth in FIG. 2. In one aspect, the polypeptide is 403 residues in length.

The invention provides an isolated or recombinant nucleic acid comprising a nucleic acid sequence (a) having a sequence identity to SEQ ID NO:30, wherein the sequence identity is at least 85%, 90%, 95% or 98% and the sequence identities are determined by analysis with a sequence comparison algorithm or by a visual inspection; (b) having a sequence as set forth in SEQ ID NO:30; (c) that hybridizes under stringent conditions to a sequence comprising SEQ ID NO:30; or (d) encoding a polypeptide as set forth in SEQ ID NO:31. The invention provides an isolated or recombinant polypeptide comprising a sequence (a) having a sequence identity to SEQ ID NO:31, wherein the sequence identity is at least 85%, 90%, 95% or 98% and the sequence identities are determined by analysis with a sequence comparison algorithm or by a visual inspection; (b) having a sequence as set forth in SEQ ID NO:31; or (c) encoded by a nucleic acid of the invention as set forth above. In one aspect, the polypeptide functions an enzyme of the kinamycin biosynthetic pathway, in particular, this polypeptide can be gene 15 of the exemplary kinamycin biosynthetic pathway as set forth in FIG. 2. In one aspect, the polypeptide is 88 residues in length.

The invention provides an isolated or recombinant nucleic acid comprising a nucleic acid sequence (a) having a sequence identity to SEQ ID NO:32, wherein the sequence identity is at least 90%, 95% or 98% and the sequence identities are determined by analysis with a sequence comparison algorithm or by a visual inspection; (b) having a sequence as set forth in SEQ ID NO:32; (c) that hybridizes under stringent conditions to a sequence comprising SEQ ID NO:32; or (d) encoding a polypeptide as set forth in SEQ ID NO: 33. The invention provides an isolated or recombinant polypeptide comprising a sequence (a) having a sequence identity to SEQ ID NO:33, wherein the sequence identity is at least 90%, 95% or 98% and the sequence identities are determined by analysis with a sequence comparison algorithm or by a visual inspection; (b) having a sequence as set forth in SEQ ID NO:33; or (c) encoded by a nucleic acid of the invention as set forth above. In one aspect, the polypeptide functions an enzyme of the kinamycin biosynthetic pathway, in particular, this polypeptide can be gene 16 of the exemplary kinamycin biosynthetic pathway as set forth in FIG. 2. In one aspect, the polypeptide is 261 residues in length.

The invention provides an isolated or recombinant nucleic acid comprising a nucleic acid sequence (a) having a sequence identity to SEQ ID NO:34, wherein the sequence identity is at least 85%, 90%, 95% or 98% and the sequence identities are determined by analysis with a sequence comparison algorithm or by a visual inspection; (b) having a sequence as set forth in SEQ ID NO:34; (c) that hybridizes under stringent conditions to a sequence comprising SEQ ID NO:34; or (d) encoding a polypeptide as set forth in SEQ ID NO:35. The invention provides an isolated or recombinant polypeptide comprising a sequence (a) having a sequence identity to SEQ ID NO:35, wherein the sequence identity is at least 85%, 90%, 95% or 98% and the sequence identities are determined by analysis with a sequence comparison algorithm or by a visual inspection; (b) having a sequence as set forth in SEQ ID NO:35; or (c) encoded by a nucleic acid of the invention as set forth above. In one aspect, the polypeptide functions an enzyme of the kinamycin biosynthetic pathway, in particular, this polypeptide can be gene 17 of the exemplary kinamycin biosynthetic pathway as set forth in FIG. 2. In one aspect, the polypeptide is 311 residues in length.

The invention provides an isolated or recombinant nucleic acid comprising a nucleic acid sequence (a) having a sequence identity to SEQ ID NO:36, wherein the sequence identity is at least 70%, 75%, 80%, 85%, 90%, 95% or 98% and the sequence identities are determined by analysis with a sequence comparison algorithm or by a visual inspection; (b) having a sequence as set forth in SEQ ID NO:36; (c) that hybridizes under stringent conditions to a sequence comprising SEQ ID NO:36; or (d) encoding a polypeptide as set forth in SEQ ID NO:37. The invention provides an isolated or recombinant polypeptide comprising a sequence (a) having a sequence identity to SEQ ID NO: 37, wherein the sequence identity is at least 70%, 75%, 80%, 85%, 90%, 95% or 98% and the sequence identities are determined by analysis with a sequence comparison algorithm or by a visual inspection; (b) having a sequence as set forth in SEQ ID NO:37; or (c) encoded by a nucleic acid of the invention as set forth above. In one aspect, the polypeptide functions an enzyme of the kinamycin biosynthetic pathway, in particular, this polypeptide can be gene 18 of the exemplary kinamycin biosynthetic pathway as set forth in FIG. 2. In one aspect, the polypeptide is 490 residues in length.

The invention provides an isolated or recombinant nucleic acid comprising a nucleic acid sequence (a) having a sequence identity to SEQ ID NO:38, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:38; (c) that hybridizes under stringent conditions to a sequence comprising SEQ ID NO:38; or (d) encoding a polypeptide as set forth in SEQ ID NO:39. The invention provides an isolated or recombinant polypeptide comprising a sequence (a) having a sequence identity to SEQ ID NO: 39, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:39; or (c) encoded by a nucleic acid of the invention as set forth above. In one aspect, the polypeptide functions an enzyme of the kinamycin biosynthetic pathway, in particular, this polypeptide can be gene 19 of the exemplary kinamycin biosynthetic pathway as set forth in FIG. 2. In one aspect, the polypeptide is 500 residues in length.

The invention provides an isolated or recombinant nucleic acid comprising a nucleic acid sequence (a) having a sequence identity to SEQ ID NO:40, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:40; (c) that hybridizes under stringent conditions to a sequence comprising SEQ ID NO:40; or (d) encoding a polypeptide as set forth in SEQ ID NO:41. The invention provides an isolated or recombinant polypeptide comprising a sequence (a) having a sequence identity to SEQ ID NO: 41, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:41; or (c) encoded by a nucleic acid of the invention as set forth above. In one aspect, the polypeptide functions an enzyme of the kinamycin biosynthetic pathway, in particular, this polypeptide can be gene 20 of the exemplary kinamycin biosynthetic pathway as set forth in FIG. 2. In one aspect, the polypeptide is 292 residues in length.

The invention provides an isolated or recombinant nucleic acid comprising a nucleic acid sequence (a) having a sequence identity to SEQ ID NO:42, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:42; (c) that hybridizes under stringent conditions to a sequence comprising SEQ ID NO:42; or (d) encoding a polypeptide as set forth in SEQ ID NO:43. The invention provides an isolated or recombinant polypeptide comprising a sequence (a) having a sequence identity to SEQ ID NO: 43, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:43; or (c) encoded by a nucleic acid of the invention as set forth above. In one aspect, the polypeptide functions an enzyme of the kinamycin biosynthetic pathway, in particular, this polypeptide can be gene 21 of the exemplary kinamycin biosynthetic pathway as set forth in FIG. 2. In one aspect, the polypeptide is 426 residues in length.

The invention provides an isolated or recombinant nucleic acid comprising a nucleic acid sequence (a) having a sequence identity to SEQ ID NO:44, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:44; (c) that hybridizes under stringent conditions to a sequence comprising SEQ ID NO:44; or (d) encoding a polypeptide as set forth in SEQ ID NO:45. The invention provides an isolated or recombinant polypeptide comprising a sequence (a) having a sequence identity to SEQ ID NO: 45, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:45; or (c) encoded by a nucleic acid of the invention as set forth above. In one aspect, the polypeptide functions an enzyme of the kinamycin biosynthetic pathway, in particular, this polypeptide can be gene 22 of the exemplary kinamycin biosynthetic pathway as set forth in FIG. 2. In one aspect, the polypeptide is 191 residues in length.

The invention provides an isolated or recombinant nucleic acid comprising a nucleic acid sequence (a) having a sequence identity to SEQ ID NO:46, wherein the sequence identity is at least 90%, 95% or 98% and the sequence identities are determined by analysis with a sequence comparison algorithm or by a visual inspection; (b) having a sequence as set forth in SEQ ID NO:46; (c) that hybridizes under stringent conditions to a sequence comprising SEQ ID NO:46; or (d) encoding a polypeptide as set forth in SEQ ID NO: 47. The invention provides an isolated or recombinant polypeptide comprising a sequence (a) having a sequence identity to SEQ ID NO:47, wherein the sequence identity is at least 90%, 95% or 98% and the sequence identities are determined by analysis with a sequence comparison algorithm or by a visual inspection; (b) having a sequence as set forth in SEQ ID NO:47; or (c) encoded by a nucleic acid of the invention as set forth above. In one aspect, the polypeptide functions an enzyme of the kinamycin biosynthetic pathway, in particular, this polypeptide can be gene 23 of the exemplary kinamycin biosynthetic pathway as set forth in FIG. 2. In one aspect, the polypeptide is 526 residues in length.

The invention provides an isolated or recombinant nucleic acid comprising a nucleic acid sequence (a) having a sequence identity to SEQ ID NO:48, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:48; (c) that hybridizes under stringent conditions to a sequence comprising SEQ ID NO:48; or (d) encoding a polypeptide as set forth in SEQ ID NO:49. The invention provides an isolated or recombinant polypeptide comprising a sequence (a) having a sequence identity to SEQ ID NO:49, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:49; or (c) encoded by a nucleic acid of the invention as set forth above. In one aspect, the polypeptide functions an enzyme of the kinamycin biosynthetic pathway, in particular, this polypeptide can be gene 24 of the exemplary kinamycin biosynthetic pathway as set forth in FIG. 2. In one aspect, the polypeptide is 101 residues in length.

The invention provides an isolated or recombinant nucleic acid comprising a nucleic acid sequence (a) having a sequence identity to SEQ ID NO:50, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:50; (c) that hybridizes under stringent conditions to a sequence comprising SEQ ID NO:50; or (d) encoding a polypeptide as set forth in SEQ ID NO:51. The invention provides an isolated or recombinant polypeptide comprising a sequence (a) having a sequence identity to SEQ ID NO: 51, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:51; or (c) encoded by a nucleic acid of the invention as set forth above. In one aspect, the polypeptide functions an enzyme of the kinamycin biosynthetic pathway, in particular, this polypeptide can be gene 25 of the exemplary kinamycin biosynthetic pathway as set forth in FIG. 2. In one aspect, the polypeptide is 260 residues in length.

The invention provides an isolated or recombinant nucleic acid comprising a nucleic acid sequence (a) having a sequence identity to SEQ ID NO:52, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:52; (c) that hybridizes under stringent conditions to a sequence comprising SEQ ID NO:52; or (d) encoding a polypeptide as set forth in SEQ ID NO:53. The invention provides an isolated or recombinant polypeptide comprising a sequence (a) having a sequence identity to SEQ ID NO:53, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:53; or (c) encoded by a nucleic acid of the invention as set forth above. In one aspect, the polypeptide functions an enzyme of the kinamycin biosynthetic pathway, in particular, this polypeptide can be gene 26 of the exemplary kinamycin biosynthetic pathway as set forth in FIG. 2. In one aspect, the polypeptide is 134 residues in length.

The invention provides an isolated or recombinant nucleic acid comprising a nucleic acid sequence (a) having a sequence identity to SEQ ID NO:54, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:54; (c) that hybridizes under stringent conditions to a sequence comprising SEQ ID NO:54; or (d) encoding a polypeptide as set forth in SEQ ID NO:55. The invention provides an isolated or recombinant polypeptide comprising a sequence (a) having a sequence identity to SEQ ID NO: 55, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:55; or (c) encoded by a nucleic acid of the invention as set forth above. In one aspect, the polypeptide functions an enzyme of the kinamycin biosynthetic pathway, in particular, this polypeptide can be gene 27 of the exemplary kinamycin biosynthetic pathway as set forth in FIG. 2. In one aspect, the polypeptide is 432 residues in length.

The invention provides an isolated or recombinant nucleic acid comprising a nucleic acid sequence (a) having a sequence identity to SEQ ID NO:56, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:56; (c) that hybridizes under stringent conditions to a sequence comprising SEQ ID NO:56; or (d) encoding a polypeptide as set forth in SEQ ID NO:57. The invention provides an isolated or recombinant polypeptide comprising a sequence (a) having a sequence identity to SEQ ID NO: 57, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:57; or (c) encoded by a nucleic acid of the invention as set forth above. In one aspect, the polypeptide functions an enzyme of the kinamycin biosynthetic pathway, in particular, this polypeptide can be gene 28 of the exemplary kinamycin biosynthetic pathway as set forth in FIG. 2. In one aspect, the polypeptide is 500 residues in length.

The invention provides an isolated or recombinant nucleic acid comprising a nucleic acid sequence (a) having a sequence identity to SEQ ID NO:58, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:58; (c) that hybridizes under stringent conditions to a sequence comprising SEQ ID NO:58; or (d) encoding a polypeptide as set forth in SEQ ID NO:59. The invention provides an isolated or recombinant polypeptide comprising a sequence (a) having a sequence identity to SEQ ID NO:59, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:59; or (c) encoded by a nucleic acid of the invention as set forth above. In one aspect, the polypeptide functions an enzyme of the kinamycin biosynthetic pathway, in particular, this polypeptide can be gene 29 of the exemplary kinamycin biosynthetic pathway as set forth in FIG. 2. In one aspect, the polypeptide is 461 residues in length.

The invention provides an isolated or recombinant nucleic acid comprising a nucleic acid sequence (a) having a sequence identity to SEQ ID NO:60, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:60; (c) that hybridizes under stringent conditions to a sequence comprising SEQ ID NO:60; or (d) encoding a polypeptide as set forth in SEQ ID NO:61. The invention provides an isolated or recombinant polypeptide comprising a sequence (a) having a sequence identity to SEQ ID NO:61, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:61; or (c) encoded by a nucleic acid of the invention as set forth above. In one aspect, the polypeptide functions an enzyme of the kinamycin biosynthetic pathway, in particular, this polypeptide can be gene 30 of the exemplary kinamycin biosynthetic pathway as set forth in FIG. 2. In one aspect, the polypeptide is 685 residues in length.

The invention provides an isolated or recombinant nucleic acid comprising a nucleic acid sequence (a) having a sequence identity to SEQ ID NO:62, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:62; (c) that hybridizes under stringent conditions to a sequence comprising SEQ ID NO:62; or (d) encoding a polypeptide as set forth in SEQ ID NO:63. The invention provides an isolated or recombinant polypeptide comprising a sequence (a) having a sequence identity to SEQ ID NO: 63, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:63; or (c) encoded by a nucleic acid of the invention as set forth above. In one aspect, the polypeptide functions an enzyme of the kinamycin biosynthetic pathway, in particular, this polypeptide can be gene 31 of the exemplary kinamycin biosynthetic pathway as set forth in FIG. 2. In one aspect, the polypeptide is 133 residues in length.

The invention provides an isolated or recombinant nucleic acid comprising a nucleic acid sequence (a) having a sequence identity to SEQ ID NO:64, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:64; (c) that hybridizes under stringent conditions to a sequence comprising SEQ ID NO:64; or (d) encoding a polypeptide as set forth in SEQ ID NO:65. The invention provides an isolated or recombinant polypeptide comprising a sequence (a) having a sequence identity to SEQ ID NO: 65, wherein the sequence identity is at least 65%, 70%, 75%, 80%, 85%, 90%, 95% or 98% and the sequence identities are determined by analysis with a sequence comparison algorithm or by a visual inspection; (b) having a sequence as set forth in SEQ ID NO:65; or (c) encoded by a nucleic acid of the invention as set forth above. In one aspect, the polypeptide functions an enzyme of the kinamycin biosynthetic pathway, in particular, this polypeptide can be gene 32 of the exemplary kinamycin biosynthetic pathway as set forth in FIG. 2. In one aspect, the polypeptide is 213 residues in length.

The invention provides a polyketide comprising a glycosylated kinamycin made by a process comprising the following steps: (a) providing a plurality of nucleic acid coding sequences, wherein the nucleic acid coding sequences have at least 95% sequence identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO: 44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62 and SEQ ID NO:64, wherein the plurality of nucleic acid coding sequences, when expressed in a Streptococcus, results in the synthesis of a kinamycin molecule in the Streptococcus; (b) providing (i) a Streptococcus sp. or a Dactylosporangium sp. bacillus, or (ii) an intracellular extract of a Streptococcus sp. or a Dactylosporangium sp.; and (c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated polyketide comprising a glycosylated kinamycin.

The invention provides methods for making a glycosylated kinamycin comprising the following steps: (a) providing a plurality of nucleic acid coding sequences, wherein the nucleic acid coding sequences have at least 95% sequence identity to SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO: 24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO: 46, SEQ ID NO:48, SEQ ID NO:50, SEQ ID NO:52, SEQ ID NO:54, SEQ ID NO:56, SEQ ID NO:58, SEQ ID NO:60, SEQ ID NO:62 and SEQ ID NO:64, wherein the plurality of nucleic acid coding sequences, when expressed in a Streptococcus, results in the synthesis of a kinamycin molecule in the Streptococcus; (b) providing (i) a Streptococcus sp. or a Dactylosporangium sp. bacillus, or (ii) an intracellular extract of a Streptococcus sp. or a Dactylosporangium sp.; and (c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated kinamycin.

The invention provides a polyketide comprising a glycosylated kinamycin made by a process comprising the following steps: (a) providing a plurality of polypeptides, wherein the polypeptide sequences have at least 95% sequence identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO: 25, SEQ ID NO:27, SEQ ID NO:29, SEQ ID NO:31, SEQ ID NO:33, SEQ I D NO: 35, SEQ ID NO:37, SEQ ID NO:39, SEQ ID NO:41, SEQ ID NO:43, SEQ ID NO:45, SEQ ID NO: 47, SEQ ID NO:49, SEQ ID NO:51, SEQ ID NO:53, SEQ ID NO:55, SEQ ID NO:57, SEQ ID NO:59, SEQ ID NO:61, SEQ ID NO:63 and SEQ ID NO:65, wherein the plurality of polypeptides, when expressed in a Streptococcus, results in the synthesis of a kinamycin molecule in the Streptococcus; (b) providing (i) a Streptococcus sp. or a Dactylosporangium sp. bacillus, or (ii) an intracellular extract of a Streptococcus sp. or a Dactylosporangium sp.; and (c) inserting the polypeptides of step (a) into the bacillus of step (b) or contacting the polypeptides of step (a) with the intracellular extract of step (b) under conditions allowing synthesis of a glycosylated kinamycin.

In one aspect, one, some or all of the nucleic acids of the invention are assembled in one or more expression cassettes, e.g., vectors. In one aspect, the coding sequences of the, invention are under the control of transcriptional regulatory sequences, e.g., promoters and enhancers.

The invention provides methods for making a glycosylated kinamycin comprising the following steps: (a) providing a plurality of polypeptides, wherein the polypeptides he sequences at least 95% sequence identity to SEQ ID NO:3, SEQ ID NO:5, SEQ ID NO:7, SEQ ID NO:9, SEQ ID NO:11, SEQ ID NO:13, SEQ ID NO:15, SEQ ID NO: 17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO: 39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO: 61, SEQ ID NO:63 and SEQ ID NO:65, wherein the plurality of polypeptides, when expressed in a Streptococcus, results in the synthesis of a kinamycin molecule in the Streptococcus; (b) providing (i) a Streptococcus sp. or a Dactylosporangium sp. bacillus, or (ii) an intracellular extract of a Streptococcus sp. or a Dactylosporangium sp.; and (c) inserting the polypeptides of step (a) into the bacillus of step (b) or contacting the polypeptides of step (a) with the intracellular extract of step (b) under conditions allowing synthesis of a glycosylated kinamycin.

The invention provides methods for making a glycosylated kinamycin that can comprise a combination of aspects of the invention, e.g., adding to a bacterial extract some coding sequences and some polypeptides to make a glycosylated kinamycin. The kinamycin or precursors of the kinamycin also can be completely or partially synthetically synthesized.

In all of the methods and processes of the invention, any appropriate Streptococcus sp. can be used, e.g., a S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus, a S. violaceoruber, or a S. diversa. The Dactylosporangium sp. of step (b) can be a Dactylosporangium sp. ATCC 53693.

The invention provides antibodies (e.g., monoclonal or polyclonal) that are capable of specifically binding to a polypeptide, wherein the polypeptide can have a sequence comprising at least 95% sequence identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO: 29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO: 51, SEQ ID NO:53, SEQ ID NO:55, SEQ ID NO:57, SEQ ID NO:59, SEQ ID NO:61, SEQ ID NO:63 and SEQ ID NO:65.

All publications, patents, patent applications, GenBank sequences and ATCC deposits, cited herein are hereby expressly incorporated by reference for all purposes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the general structure of an exemplary glycosylated kinamycin of the invention.

FIG. 2 is a schematic of the S. murayamaensis nucleic acid insert of ATCC 21414, and the 32 coding sequences encoded therein which, when expressed in a bacterial system, produces kinamycin.

FIG. 3 is a schematic of an exemplary biosynthetic pathway for making kinamycin and includes the structures of exemplary compounds of the invention.

Like reference symbols in the various drawings indicate like elements.

### DETAILED DESCRIPTION

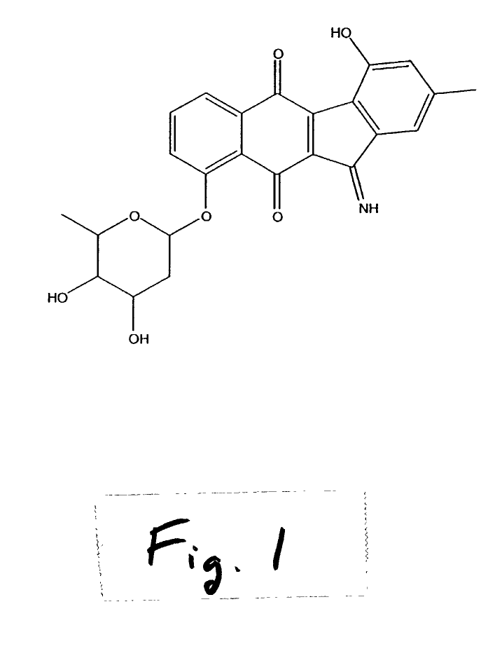

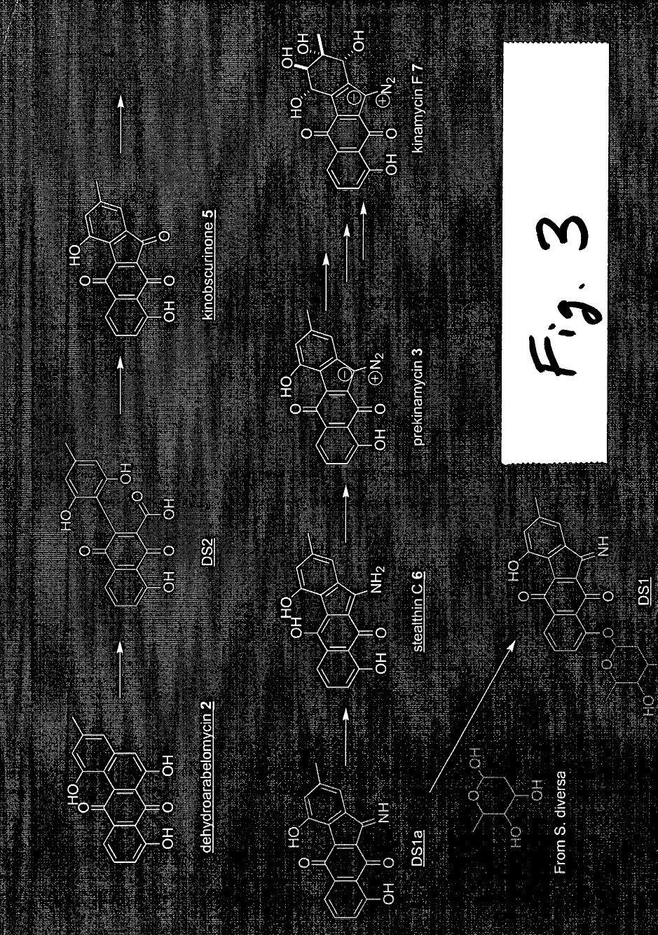

The compositions and methods of the invention can be used to treat infections, i.e., as antibiotics, and as anti-tumor agents. The compositions of the invention can also be used as electrophilic azo-coupling agents in vitro or in vivo. This invention provides novel glycosylated kinamycins that are useful as antibiotics and anti-tumor agents. Compositions of the invention comprise glycosylated kinamycins. The compositions and methods of the invention can be used to treat infections. In one aspect, the invention provides glycosylated polycyclic aromatic quinone antibiotics as shown in FIG. 1 and FIG. 3. These structures can be glycosylated in a variety of forms.

The invention also provides enzymes capable of generating kinamycin, nucleic acids that encode them, antibodies that bind to them, and methods for making and using them.

In one aspect, the compound of the invention is manufactured by first making a kinamycin and then glycosylating it. For example, the kinamycin can be made by growing a sample of Streptomyces murayamaensis as described, e.g., in J. Antibiotics, 23:315 (1970) and then isolating the kinamycin. Alternatively, the kinamycin can be made synthetically. The isolated or synthesized kinamycin can then be glycosylated, e.g., derivatized to form the compound in FIG. 1.

The compositions of the invention can be used as conventional antibiotics, e.g., as pharmaceuticals in the treatment of infections. For example, the compound can be given to a subject, e.g., a patient or other subject, suffering from a bacterial infection. Administration of the compound can be through any means, e.g., any conventional methods, e.g., oral, intravenous, intradermal, parenteral, transdermal or other methods.

In one aspect, the compound is used as a research tool to inhibit the growth of bacteria in vitro. For example, a bacterial colony can be plated onto agar that incorporates a growth-inhibiting quantity of a compound of the invention, e.g., the kinamycin derivative shown in FIG. 1. Bacteria that are resistant to the compound of the invention will continue to grow, while bacteria that are not resistant will be inhibited from growing.

The invention is not limited to the exemplary structure shown in FIG. 1. The invention provides similar structures that provide the antibacterial effects, as described herein.

One of ordinary skill in the art can test for antibacterial effects by growing (e.g., culturing) bacteria, such as Micrococcus luteus, in the presence and absence of a compound of the invention, e.g., a derivatized glycosylated kinamycin compound. Those compounds that inhibit the growth of the bacteria are selected for further tests, while those compounds that do not inhibit bacterial growth are not selected for further tests.

Definitions

To facilitate understanding the invention, a number of terms are defined below. Unless defined otherwise, all technical and scientific terms used herein have the meaning commonly understood by a person skilled in the art to which this invention belongs.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

As used herein, “isolated,” when referring to a molecule or composition, such as, for example, a glycosylated kinamycin of the invention, means that the molecule or composition is separated from at least one other compound, such as a protein, other nucleic acids (e.g., RNAs, polypeptides, small compounds), or other contaminants with which it is associated in vivo or in its naturally occurring state. Thus, a compound is considered isolated when it has been isolated from any other component with which it is naturally associated, e.g., cell membrane, as in a cell extract, serum, and the like. An isolated composition can, however, also be substantially pure. An isolated composition can be in a homogeneous state and can be in a dry or an aqueous solution. Purity and homogeneity can be determined, for example, using analytical chemistry techniques such as polyacrylamide gel electrophoresis (SDS-PAGE) or high performance liquid chromatography (HPLC).

The term “expression cassette” as used herein refers to a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The term “chemically linked” refers to any chemical bonding of two moieties, e.g., a kinamycin of the invention and a saccharide or polysaccharide. The saccharide and kinamycin moieties of the compounds of the invention can be chemically linked by any means known in the art.

The term “pharmaceutical composition” refers to a composition suitable for pharmaceutical use, e.g., as an antibiotic or an anti-cancer agent comprising a glycosylated kinamycin of the invention, in a subject. The pharmaceutical compositions of this invention are formulations that comprise a pharmacologically effective amount of a composition comprising, e.g., a glycosylated kinamycin of the invention, and a pharmaceutically acceptable carrier.

The term “promoter” is an array of nucleic acid control sequences which direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. The nucleic acids of the invention can be operatively linked to any type of promoter (or transcriptional regulatory sequence) alone or in an expression cassette, e.g., a vector.

The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

“Amino acid” or “amino acid sequence” as used herein refer to an oligopeptide, peptide, polypeptide, or protein sequence, or to a fragment, portion, or subunit of any of these, and to naturally occurring or synthetic molecules.

The term “polypeptide” as used herein, refers to amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The peptides and polypeptides of the invention also include all “mimetic” and “peptidomimetic” forms, as described in further detail, below.

The phrase “substantially identical” in the context of two nucleic acids or polypeptides, refers to two or more sequences that have at least 60%, 65% 70%, 75%, 80%, 85%, 90%, 95%, 98% or 99% nucleotide or amino acid residue (sequence) identity (or as otherwise set forth herein), when compared and aligned for maximum correspondence, as measured using one any known sequence comparison algorithm or by visual inspection. In alternative aspects, the invention provides nucleic acid and polypeptide sequences having substantial identity to an exemplary sequence of the invention over a region of at least about 100 residues, 150 residues, 200 residues, 250 residues, 300 residues, 350 residues, or over a region ranging from between about 50 residues to the full length of the nucleic acid or polypeptide. Nucleic acid sequences of the invention can be substantially identical over the entire length of a polypeptide coding region. The invention provides polypeptides that are “substantially identical” to the exemplary amino acid sequences of the invention. “Substantially identical” can be a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted resulting in modification of the structure of the polypeptide, without significantly altering its biological activity. For example, amino- or carboxyl-terminal amino acids that are not required for enzymatic activity can be removed. Modified polypeptide sequences of the invention can be assayed for activity by any number of methods, including contacting the modified polypeptide sequence with a substrate and determining whether the modified polypeptide decreases the amount of specific substrate in the assay or increases the bioproducts of the enzymatic reaction of a functional enzyme with the substrate, as discussed herein and illustrated in the Figures.

“Hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature, as described in detail, below.

General Synthetic Synthesis Methods

The present invention provides a novel genus of glycosylated kinamycin compounds that have antibiotic and anti-tumor activities. The skilled artisan will recognize that the compositions of the invention (and their precursors) can be synthesized using a variety of procedures and methodologies, which are well described in the scientific and patent literature., e.g., Organic Syntheses Collective Volumes, Gilman et al. (Eds) John Wiley & Sons, Inc., NY; Venuti (1989) Pharm Res. 6:867-873. The invention can be practiced in conjunction with any method or protocol known in the art, which are well described in the scientific and patent literature.

Generating and Manipulating Nucleic Acids

The invention provides nucleic acids, including expression cassettes such as expression vectors, encoding the polypeptides of the invention. The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

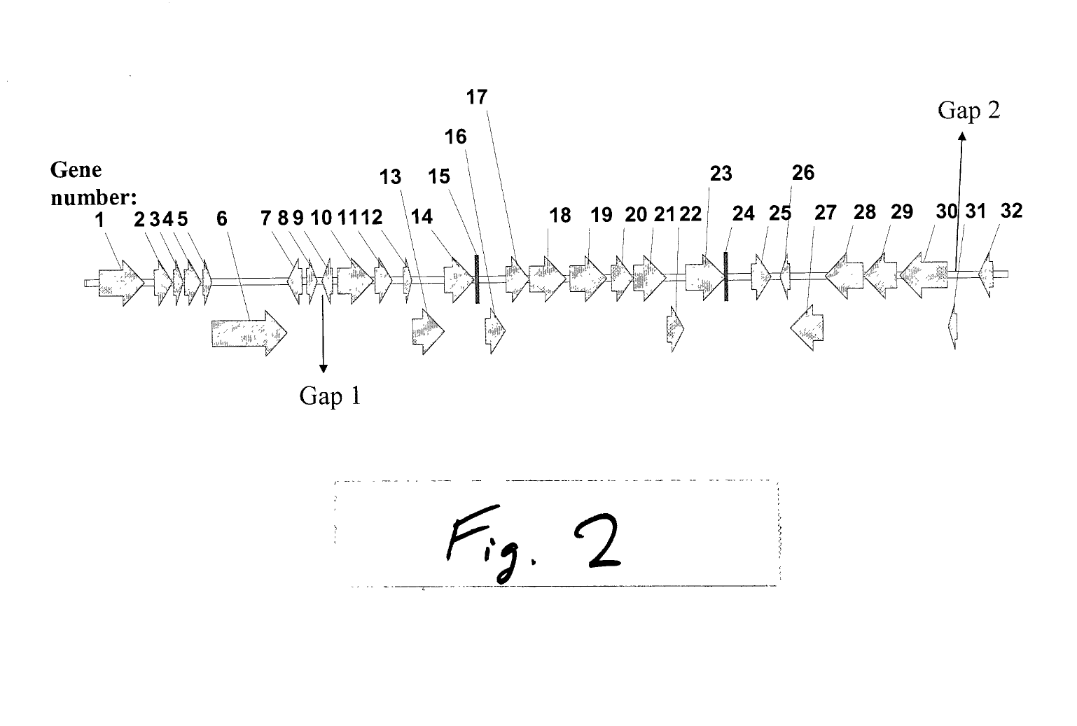

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention can include genomic or cDNA libraries. The libraries, or any individual or collective nucleic acid sequences of the invention, can be contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids. For example, the nucleic acid sequences of the invention can be assembled in any of these expression systems as set forth in FIG. 2, or any other order, to express translation product and to generate a composition of the invention.

In one aspect, a nucleic acid encoding a polypeptide of the invention is assembled in appropriate phase with a leader sequence capable of directing secretion of the translated polypeptide or fragment thereof. The invention also provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides DNA sequences of the invention operatively linked to expression (e.g., transcriptional or translational) control sequence(s),. e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter.

Expression Vectors and Cloning Vehicles

The invention provides expression vectors and cloning vehicles comprising nucleic acids of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector may comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells may also contain enhancers to increase expression levels. Enhancers are cis-acting elements of DNA, can be about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

A DNA sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel and Sambrook. Such procedures and others are deemed to be within the scope of those skilled in the art.

The vector may be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

Host Cells and Transformed Cells

The invention also provides a transformed cell comprising a nucleic acid sequence of the invention, including an expression cassette of the invention (e.g., a vector or BAC comprising a coding sequence of the invention). The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Amplification of Nucleic Acids

In practicing the invention, nucleic acids encoding the polypeptides of the invention, or modified nucleic acids, can be reproduced by, e.g., amplification. Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Determining the Degree of Sequence Identity and Identifying Motifs

The invention provides nucleic acids and polypeptides having various % sequence identities (as set forth herein) to the exemplary nucleic acids and polypeptides of the invention. The extent of sequence identity (homology) may be determined using any computer program and associated parameters, including those described herein, such as FASTA version 3.0t78, or, BLAST, with the default parameters. Homologous sequences also include RNA sequences in which uridines replace the thymines in the nucleic acid sequences. The homologous sequences may be obtained using any of the procedures described herein or may result from the correction of a sequencing error. It will be appreciated that the nucleic acid sequences as set forth herein can be represented in the traditional single character format (see, e.g., Stryer, Lubert. Biochemistry, 3rd Ed., W. H Freeman & Co., New York) or in any other format which records the identity of the nucleotides in a sequence.

To determine and identify sequence identities, structural homologies, motifs and the like in silico the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention. Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art. Aspects of the invention include systems (e.g., internet based systems), particularly computer systems, which store and manipulate the sequences and sequence information.

Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993). Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence of the invention) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous residues. For example, in alternative aspects of the invention, continugous residues ranging anywhere from 20 to the full length of one or more exemplary sequences are compared to a reference sequence of the same number of contiguous positions after the two sequences are optimally aligned. If the reference sequence has the requisite sequence identity to an exemplary sequence that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las,Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Several genomes have been sequenced, e.g., M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000).

One algorithm that can be used to determine if a sequence is within the scope of the invention is BLAST and BLAST 2.0 algorithms, which are described in Altschul et al., Nuc. Acids Res. 25:3389-3402, 1977, and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). The parameters used with the above algorithms may be adapted depending on the sequence length and degree of homology studied. In some embodiments, the parameters may be the default parameters used by the algorithms in the absence of instructions from the user.

Hybridization of Nucleic Acids

The invention provides isolated or recombinant nucleic acids that hybridize under stringent conditions to an exemplary sequence of the invention. The stringent conditions can be highly stringent conditions, medium stringent conditions and low stringent conditions. In alternative embodiments, nucleic acids of the invention as defined by their ability to hybridize under stringent conditions can be between about five residues and the full length of an exemplary sequence of the invention. For example, they can be at least 5, 10, 15, 20, 25, 30, 35, 40, 50, 55, 60, 65, 70, 75, 80, 90, 100 and 150 residues in length. Nucleic acids shorter than full length are also included. These nucleic acids are useful as, e.g., hybridization probes, labeling probes, PCR oligonucleotide probes, sequences encoding antibody binding peptides (epitopes), motifs, active sites and the like.

In one aspect, hybridization under high stringency conditions is in about 50% formamide at about 37° C. to 42° C. Hybridization also can be under reduced stringency conditions in about 35% to 25% formamide at about 30° C. to 35° C. Alternatively, hybridization can be under high stringency conditions at 42° C. in 50% formamide, 5× SSPE, 0.3% SDS, and 200 n/ml sheared and denatured salmon sperm DNA. Hybridization can also be under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following exemplary formulas. For probes between 14 and 70 nucleotides in length the melting temperature (Tm) is calculated using the formula: Tm=81.5+16.6(log [Na+])+0.41 (fraction G+C)−(600/N) where N is the length of the probe. If the hybridization is carried out in a solution containing formamide, the melting temperature may be calculated using the equation: Tm=81.5+16.6(log [Na+])+0.41(fraction G+C)−(0.63% formamide)−(600/N) where N is the length of the probe. Prehybridization may be carried out in 6× SSC, 5× Denhardt's reagent, 0.5% SDS, 100 μg denatured fragmented salmon sperm DNA or 6× SSC, 5× Denhardt's reagent, 0.5% SDS, 100 μg denatured fragmented salmon sperm DNA, 50% formamide. Formulas for SSC and Denhardt's and other solutions are listed, e.g., in Sambrook.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2× SSC at a temperature of at least about 50° C. or about 55° C. to about 60° C. for about 15 to about 20 minutes; or, the hybridization complex is washed twice with a solution with a salt concentration of about 2X SSC containing 0. 1% SDS at room temperature for 15 minutes and then washed twice by 0.1X SSC containing 0.1% SDS at 68° C. for 15 minutes; or, equivalent conditions. See Sambrook, Tijssen and Ausubel for a description of SSC buffer and equivalent conditions.

Oligonucleotides Probes and Methods for Using Them

The invention also provides nucleic acid probes for identifying nucleic acids encoding a polypeptide of the invention. In one aspect, the probe comprises at least 10 consecutive bases of an exemplary sequence. Alternatively, a probe of the invention can be at least about 5, 6, 7, 8 or 9 to about 40, about 10 to 50, about 20 to 60 about 30 to 70, consecutive bases of an exemplary sequence. The probes identify a nucleic acid by binding or hybridization. The probes can be used in arrays, including, e.g., capillary arrays. The probes of the invention can also be used to isolate other nucleic acids or polypeptides.

The probes of the invention can be used to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences present in the sample. Where necessary, conditions which permit the probe to specifically hybridize to complementary sequences may be determined by placing the probe in contact with complementary sequences from samples known to contain the complementary sequence, as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids (see discussion on specific hybridization conditions).

Polypeptides and Peptides

The invention provides polypeptides involved in the synthesis of polyketides, e.g., kinamycin, and subsequences thereof, e.g., peptides. This invention provides immunogenic peptides capable of generating an immune response, e.g., antibodies. Polypeptides and peptides of the invention can be isolated from natural sources (e.g., bacteria), be synthetic, or be recombinantly generated polypeptides. Peptides and proteins can be recombinantly expressed in vitro or in vivo. The peptides and polypeptides of the invention can be made and isolated using any method known in the art, and the invention provides a few exemplary means for generating such proteins.

The polypeptides of the invention include “mimetics” and “peptidomimetics,” which are synthetic chemical compounds that have substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues; non-natural residues are well described in the scientific and patent literature.

Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and. Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Antibody Generation

The invention provides antibodies that specifically bind to the polypeptides of the invention. The polypeptides or peptide can be conjugated to another molecule or can be administered with an adjuvant. The coding sequence can be part of an expression cassette or vector capable of expressing the immunogen in vivo. (see, e.g., Katsumi (1994) Hum. Gene Ther. 5:1335-9). Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press,

New York, N.Y. (1986); Kohler (1975) Nature 256:495; Harlow (1988) ANTIBODIES, A LABORATORY MANUAL, Cold Spring Harbor Publications, New York.

Antibodies also can be generated in vitro, e.g., using recombinant antibody binding site expressing phage display libraries, in addition to the traditional in vivo methods using animals. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45.

Formulation and Administration of Pharmaceutical Compositions

In one embodiment, the compositions of the invention are combined with a pharmaceutically acceptable carrier (excipient) to form a pharmacological composition. The compositions of the invention can be used as antibiotics or as anti-tumor agents.

Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of a composition of the invention is dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions of the invention, when administered orally, can be protected from digestion. This can be accomplished either by complexing the composition with a compound to render it resistant to acidic and enzymatic hydrolysis or by packaging the peptide or complex in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani (1996) “Systemic delivery of peptides and proteins across absorptive mucosae” Crit. Rev. Ther. Drug Carrier Syst. 13:85-184. For topical, transdermal administration, the agents are formulated into ointments, creams, salves, powders and gels. Transdermal delivery systems can also include, e.g., patches.

The peptides and polypeptide complexes can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney (1998) Nat. Biotechnol. 16:153-157).

For inhalation, the peptide or polypeptide can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Elan Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another embodiment, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the complexes in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The compositions used in the methods of the invention can be delivered alone or as pharmaceutical compositions by any means known in the art, e.g., systemically, regionally, or locally (e.g., directly into, or directed to, a tumor); by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver a peptide or polypeptide complex of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's,. See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising compositions of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble peptides or complexes have been attached to the surface of the monolayer or bilayer. For example, compositions of the invention can be attached to peptides and peptides can be attached to hydrazide-PEG-(distearoylphosphatidyl) ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the peptides and/or complexes of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467, U.S. Pat. Nos. 4, 235,871, 4,501,728 and 4,837,028.

Treatment Regimens: Pharmacokinetics

The pharmaceutical compositions of the invention can be administered in a variety of unit dosage forms depending upon the method of administration and objective, e.g., as an antibiotic or anti-tumor agent. Dosages for typical polyketide-comprising pharmaceutical compositions are well known to those of skill in the art. Such dosages are typically advisorial in nature and are adjusted depending on the particular therapeutic context, patient tolerance, etc. The amount of composition of the invention adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton (1997) “Bioavailability and transport of peptides and peptide drugs into the brain” Peptides 18:1431-1439; Langer (1990) Science 249:1527-1533.

In one aspect for therapeutic application, compositions of the invention can be administered to a subject suffering from an infection in an amount sufficient to at least partially arrest the infection and/or its complications. For example, in one aspect, a soluble pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

### EXAMPLES

### Example 1

Expression of Kinamycin Pathway in Streptococcus

Construction of a S. murayamaensis ATCC 21414 Library

The nucleic acid of ATCC 21414, when expressed in a bacterial system, produces kinamycin, a type II polyketide. The genomic DNA of ATCC 21414 was isolated and digested with Sau3A and ligated to the BamHI-cut pMF17 fosmid and lambda-packaged. The packaged library was transfected into an E. coli STR611. The fosmid was constructed by putting together the following sequences: a. FOS 1 replicon, for maintenance in E. coli; b. apramycin-resistance gene, for selection; c. Chloramphenicol resistance gene, for selection; d. attP, for integration; e. oriT, allows transfer from E. coli to Streptomyces.

Construction of the PKS probes

Primers were designed to identify and amplify PKS genes from Actinomycetes as described previously (see, e.g., M. Metsa-Ketela et al, FEMS Microbiology Letters, 180(1999)1-6). A 612 base pair (bp) PCR fragment was amplified from the genomic DNA of S. murayamaensis, which was then sequenced. The 612 bp DNA sequence showed high sequence similarity to jadomycin PKS genes from S. venezuelae. The PKS fragment was used as a probe for colony blot hybridization of the ATCC 21414 library. 18 clones were obtained that hybridized strongly to the PKS probe.

RFLP Data

All the clones which hybridized to the PKS probe shared common bands suggesting that contigs of the genomic DNA containing the PKS genes had been cloned.

DS4 is a S. diversa strain in which both chloramphenicol and jadomycin pathways have been knocked out. The PKS positive clones #1-18 were introduced into DS4 strain by E. coli-Streptomyces mating procedure. Clones 1-6 gave exconjugants which produced green diffusible pigment, with clones 5 and 6 producing a lighter green pigment. Clones 8, 11, 16, and 18 gave exconjugants that phenotypically looked similar to the exconjugants obtained using the fosmid alone, while clones 9, 12-15 failed to give any exconjugants. When the DS4 exconjugant clones were bioassayed against M. luteus, clones 1-6 (which produced the green diffusible pigment), showed bioactivity while all others were negative.

Mating into S. coelicolor M512

S. coelicolor M512 is a strain derived from S. coelicolor A3(2) in which the resident pathways actinorhodin, undecylprodigiosin, and methylenomycin have been eliminated/blocked. When E. coli clones containing the PKS positive fosmids #1 and #2 were mated into M512, exconjugants were obtained Which showed green diffusible pigment similar to that observed in S. diversa. These clones were not bioassayed.

Chemistry

One bioactive clone S. diversa kin-1 was chemically characterized to identify the nature of the active molecule. The structure of the novel glycosylated molecule is shown in FIG. 2.

Sequence Analysis

The insert present in the fosmid DNA (from one bioactive clone called kin-1) was sequenced. There are two gaps still remaining which are perhaps a few hundred base pairs each. The sequence analysis (BLAST) showed a similarity to jadomycin biosynthetic gene cluster in the organization of the PKS and other modifying genes. There were no sugar genes or glycosyltransferases detected in the kinamycin gene cluster. Therefore, the sugar moiety in the novel glycosylated molecule is most likely derived from an endogenous Streptococcus pathway.

A number of embodiments of the invention have been described. Nevertheless, it will be understood that various modifications may be made without departing from the spirit and scope of the invention. Accordingly, other embodiments are within the scope of the following claims.

What is claimed is:

1. An isolated polyketide, wherein the polyketide comprises a kinamycin comprising least one saccharide moiety.

2. The polyketide of claim 1, wherein the saccharide comprises a polysaccharide.

3. The polyketide of claim 1, wherein the saccharide comprises a 2, 6 dideoxysugar.

4. The polyketide of claim 3, wherein the 2, 6 dideoxysugar comprises a digitose.

5. The polyketide of claim 4, wherein the digitose comprises an L-digitose.

6. The polyketide of claim 1, wherein the saccharide comprises an olivose.

7. The polyketide of claim 1, wherein the saccharide comprises a lactose, a galactose, a glucose or a fructose.

8. The polyketide of claim 1, wherein the polyketide comprises a type II polyketide.

9. The polyketide of claim 8, wherein the type II polyketide comprises a kinamycin.

10. The polyketide of claim 9, wherein the kinamycin comprises an aglycone kinamycin.

11. An isolated kinamycin molecule, wherein the kinamycin molecule comprises at least one saccharide moiety.

12. A pharmaceutical composition comprising a polyketide, wherein the polyketide comprises a kinamycin comprising at least one saccharide moiety, and a pharmaceutically acceptable carrier.

13. A pharmaceutical composition comprising a kinamycin, wherein the kinamycin comprises at least one saccharide moiety, and a pharmaceutically acceptable carrier.

14. The pharmaceutical composition of claim 12 or claim 13, wherein the pharmaceutically acceptable carrier comprises a solid or a liquid.

15. The pharmaceutical composition of claim 12 or claim 13, wherein the saccharide comprises a polysaccharide.

16. The pharmaceutical composition of claim 12 or claim 13, wherein the saccharide comprises a 2, 6 dideoxysugar.

17. The pharmaceutical composition of claim 16, wherein the 2, 6 dideoxysugar comprises a digitose.

18. The pharmaceutical composition of claim 17, wherein the digitose comprises an L-digitose.

19. The pharmaceutical composition of claim 12 or claim 13, wherein the saccharide comprises an olivose.

20. The pharmaceutical composition of claim 12 or claim 13, wherein the polyketide comprises a type II polyketide.

21. The pharmaceutical composition of claim 13, wherein the kinamycin is an aglycone kinamycin.

22. A polyketide comprising a glycosylated kinamycin made by a process comprising the following steps:
(a) providing a nucleic acid comprising a Streptococcus murayamaensis nucleic acid sequence comprising an insert deposited as ATCC accession no. ______;
(b) providing (i) a Streptococcus sp. or a Dactylosporangium sp. bacillus, or (ii) an intracellular extract of a Streptococcus sp. or a Dactylosporangium sp.; and
(c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated polyketide comprising a glycosylated kinamycin.

23. The polyketide of claim 22, further comprising isolating the glycosylated kinamycin.

24. The polyketide of claim 22, wherein the polyketide comprises a type II polyketide.

25. The polyketide of claim 22, wherein the glycosylation comprises a saccharide.

26. The polyketide of claim 25, wherein the saccharide further comprises a polysaccharide.

27. The polyketide of claim 25, wherein the saccharide comprises a 2, 6 dideoxysugar.

28. The polyketide of claim 26, wherein the 2, 6 dideoxysugar comprises a digitose.

29. The polyketide of claim 28, wherein the digitose comprises an L-digitose.

30. The polyketide of claim 25, wherein the saccharide comprises an olivose.

31. The polyketide of claim 25, wherein the saccharide comprises a lactose, a galactose, a glucose or a fructose.

32. The polyketide of claim 22, wherein the kinamycin is an aglycone kinamycin.

33. The polyketide of claim 22, wherein the Streptococcus sp. of step (b) is selected from the group consisting of S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus and S. violaceoruber.

34. The polyketide of claim 22, wherein the Dactylosporangium sp. of step (b) is a Dactylosporangium sp. ATCC 53693.

35. An isolated glycosylated kinamycin made by a process comprising the following steps:
(a) providing a nucleic acid comprising a Streptococcus murayamaensis nucleic acid sequence comprising an insert deposited as ATCC accession no. ______;
(b) providing (i) a Streptococcus sp. bacillus, or (ii) an intracellular extract of a Streptococcus sp.; and
(c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated kinamycin.

36. A method for making a composition comprising a glycosylated kinamycin comprising the following steps:
(a) providing a nucleic acid comprising a Streptococcus murayamaensis nucleic acid sequence comprising an insert deposited as ATCC accession no. ______;
(b) providing (i) a Streptococcus sp. or a Dactylosporangium sp. bacillus, or (ii) an intracellular extract of a Streptococcus sp. or a Dactylosporangium sp.;
(c) inserting the nucleic acid into the Streptococcus sp. of step (b) or contacting the nucleic acid with the intracellular extract of step (b) under conditions wherein the nucleic acid is transcribed into transcription products and the transcription products are translated into polypeptides, thereby making a glycosylated kinamycin.

37. The method of claim 36, further comprising isolating the glycosylated kinamycin.

38. The method of claim 36, wherein the polyketide comprises a type II polyketide.

39. The method of claim 36, wherein the glycosylation comprises a saccharide.

40. The method of claim 39, wherein the saccharide further comprises a polysaccharide.

41. The method of claim 39, wherein the saccharide comprises a 2, 6 dideoxysugar.

42. The method of claim 41, wherein the 2, 6 dideoxysugar comprises a digitose.

43. The method of claim 42, wherein the digitose comprises an L-digitose.

44. The method of claim 39, wherein the saccharide comprises an olivose.

45. The method of claim 39, wherein the saccharide comprises a lactose, a galactose, a glucose or a fructose.

46. The method of claim 36, wherein the kinamycin is an aglycone kinamycin.

47. The method of claim 36, wherein the Streptococcus sp. of step (b) is selected from the group consisting of S. peuceticus, S. griseus, S. peuceticus var. caesius, S. nogalater, S. galilaeus, S. argillaceus, S. atroolivaceus, S. olivoreticuli, S. cyanogenus, S. globisporus, S. fradiae, Actinomadura hibisca, S. olivaceus and S. violaceoruber.

48. The method of claim 36, wherein the Dactylosporangium sp. of step (b) is a Dactylosporangium sp. ATCC 53693.

49. An isolated composition comprising a compound having a general formula as set forth as DS2 in FIG. 3.

50. An isolated composition comprising a compound having a general formula as set forth as DS1a in FIG. 3.

51. The isolated composition of claim 48 or claim 49, further comprising a saccharide.

52. The isolated composition of claim 51, wherein the saccharide comprises a 2, 6 dideoxysugar.

53. The method of claim 52, wherein the 2, 6 dideoxysugar comprises a digitose.

54. The method of claim 53, wherein the digitose comprises an L-digitose.

55. The method of claim 51, wherein the saccharide comprises an olivose.

56. The method of claim 51, wherein the saccharide comprises a lactose, a galactose, a glucose or a fructose.

57. An isolated composition comprising a compound having a general formula as set forth as DS1 in FIG. 3.

58. An isolated or recombinant nucleic acid comprising a nucleic acid sequence having 95% sequence identity to SEQ ID NO:1, wherein the nucleic acid, when expressed in a Streptococcus, results in the synthesis of a kinamycin molecule in the Streptococcus.

59. An isolated or recombinant nucleic acid comprising a nucleic acid sequence having a sequence as set forth in SEQ ID NO:1, wherein the nucleic acid, when expressed in a Streptococcus, results in the synthesis of a kinamycin molecule in the Streptococcus.

60. An isolated or recombinant nucleic acid comprising a nucleic acid sequence
(a) having a sequence identity to SEQ ID NO:2, wherein the sequence identity is at least 85%, 90%, 95%, or 98%, and the sequence identities are determined by analysis with a sequence comparison algorithm or by a visual inspection;
(b) having a sequence as set forth in SEQ ID NO:2;
(c) that hybridizes under stringent conditions to a sequence comprising SEQ ID NO:2; or
(d) encoding a polypeptide as set forth in SEQ ID NO:3.

61. An isolated or recombinant polypeptide comprising a sequence
(a) having a sequence identity to SEQ ID NO:3, wherein the sequence identity is at least 85%, 90%, 95%, or 98%, and the sequence identities are determined by analysis with a sequence comparison algorithm or by a visual inspection;
(b) having a sequence as set forth in SEQ ID NO:3; or
(c) encoded by a nucleic acid as set forth in claim 60.

62. An isolated or recombinant nucleic acid comprising a nucleic acid sequence
(a) having a sequence identity to SEQ ID NO:4, wherein the sequence identity is at least 70%, 75%, 80%, 85%, 90%, 95%, or 98%, and the sequence identities are determined by analysis with a sequence comparison algorithm or by a visual inspection;
(b) having a sequence as set forth in SEQ ID NO:4;
(c) that hybridizes under stringent conditions to a sequence comprising SEQ ID NO:4; or
(d) encoding a polypeptide as set forth in SEQ ID NO:5.

63. An isolated or recombinant polypeptide comprising a sequence
(a) having a sequence identity to SEQ ID NO:5, wherein the sequence identity is at least 70%, 75%, 80%, 85%, 90%, 95%, or 98%, and the sequence identities are determined by analysis with a sequence comparison algorithm or by a visual inspection;
(b) having a sequence as set forth in SEQ ID NO:5; or
(c) encoded by a nucleic acid as set forth in claim 62.

64. An isolated or recombinant nucleic acid comprising-a nucleic acid sequence
(a) having a sequence identity to SEQ ID NO:6, wherein the sequence identity is at least 80%, 85%, 90%, 95%, or 98%, and the sequence identities are determined by analysis with a sequence comparison algorithm or by a visual inspection;
(b) having a sequence as set forth in SEQ ID NO:6;
(c) that hybridizes under stringent conditions to a sequence comprising SEQ ID NO:6; or
(d) encoding a polypeptide as set forth in SEQ ID NO:7.

65. An isolated or recombinant polypeptide comprising a sequence
(a) having a sequence identity to SEQ ID NO:7, wherein the sequence identity is at least 80%, 85%, 90%, 95%, or 98%, and the sequence identities are determined by analysis with a sequence comparison algorithm or by a visual inspection;
(b) having a sequence as set forth in SEQ ID NO:7; or
(c) encoded by a nucleic acid as set forth in claim 64.

66. An isolated or recombinant nucleic acid comprising a nucleic acid sequence
(a) having a sequence identity to SEQ ID NO:8, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:8;
(c) that hybridizes under stringent conditions to a sequence comprising SEQ ID NO:8; or
(d) encoding a polypeptide as set forth in SEQ ID NO:9.

67. An isolated or recombinant polypeptide comprising a sequence
(a) having a sequence identity to SEQ ID NO:9, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:9; or
(c) encoded by a nucleic acid as set forth in claim 66.

68. An isolated or recombinant nucleic acid comprising a nucleic acid sequence
(a) having a sequence identity to SEQ ID NO:10, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:10;
(c) that hybridizes under stringent conditions to a sequence comprising SEQ ID NO:10; or
(d) encoding a polypeptide as set forth in SEQ ID NO:11.

69. An isolated or recombinant polypeptide comprising a sequence
(a) having a sequence identity to SEQ ID NO:11, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:11; or
(c) encoded by a nucleic acid as set forth in claim 68.

70. An isolated or recombinant nucleic acid comprising a nucleic acid sequence
(a) having a sequence identity to SEQ ID NO:12, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:12;
(c) that hybridizes under stringent conditions to a sequence comprising SEQ ID NO:12; or
(d) encoding a polypeptide as set forth in SEQ ID NO:13.

71. An isolated or recombinant polypeptide comprising a sequence
(a) having a sequence identity to SEQ ID NO:13, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:13; or
(c) encoded by a nucleic acid as set forth in claim 70.

72. An isolated or recombinant nucleic acid comprising a nucleic acid sequence
(a) having a sequence identity to SEQ ID NO:14, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:14;
(c) that hybridizes under stringent conditions to a sequence comprising SEQ ID NO:14; or
(d) encoding a polypeptide as set forth in SEQ ID NO:15.

73. An isolated or recombinant polypeptide comprising a sequence
(a) having a sequence identity to SEQ ID NO:15, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:15; or
(c) encoded by a nucleic acid as set forth in claim 72.

74. An isolated or recombinant nucleic acid comprising a nucleic acid sequence
(a) having a sequence identity to SEQ ID NO:16, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:16;
(c) that hybridizes under stringent conditions to a sequence comprising SEQ ID NO:16; or
(d) encoding a polypeptide as set forth in SEQ ID NO:17.

75. An isolated or recombinant polypeptide comprising a sequence
(a) having a sequence identity to SEQ ID NO:17, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:17; or
(c) encoded by a nucleic acid as set forth in claim 74.

76. An isolated or recombinant nucleic acid comprising a nucleic acid sequence
(a) having a sequence identity to SEQ ID NO:18, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:18;
(c) that hybridizes under stringent conditions to a sequence comprising SEQ ID NO:18; or
(d) encoding a polypeptide as set forth in SEQ ID NO:19.

77. An isolated or recombinant polypeptide comprising a sequence
(a) having a sequence identity to SEQ ID NO:19, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:19; or
(c) encoded by a nucleic acid as set forth in claim 76.

78. An isolated or recombinant nucleic acid comprising a nucleic acid sequence
(a) having a sequence identity to SEQ ID NO:20, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:20;
(c) that hybridizes under stringent conditions to a sequence comprising SEQ ID NO:20; or
(d) encoding a polypeptide as set forth in SEQ ID NO:21.

79. An isolated or recombinant polypeptide comprising a sequence
(a) having a sequence identity to SEQ ID NO:21, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:21; or
(c) encoded by a nucleic acid as set forth in claim 78.

80. An isolated or recombinant nucleic acid comprising a nucleic acid sequence
(a) having a sequence identity to SEQ ID NO:22, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:22;
(c) that hybridizes under stringent conditions to a sequence comprising SEQ ID NO:22; or
(d) encoding a polypeptide as set forth in SEQ ID NO:23.

81. An isolated or recombinant polypeptide comprising a sequence
(a) having a sequence identity to SEQ ID NO:23, wherein the sequence identity is at least 65%, 70%, 75%, 80%, 85%, 90%, 95%, or 98%, and the sequence identities are determined by analysis with a sequence comparison algorithm or by a visual inspection;
(b) having a sequence as set forth in SEQ ID NO:23; or
(c) encoded by a nucleic acid as set forth in claim 80.

82. An isolated or recombinant nucleic acid comprising a nucleic acid sequence
(a) having a sequence identity to SEQ ID NO:24, wherein the sequence identity is at least 95% or 98% and the sequence identities are determined by analysis with a sequence comparison algorithm or by a visual inspection;
(b) having a sequence as set forth in SEQ ID NO:24;
(c) that hybridizes under stringent conditions to a sequence comprising SEQ ID NO:24; or
(d) encoding a polypeptide as set forth in SEQ ID NO:25.

83. An isolated or recombinant polypeptide comprising a sequence
(a) having a sequence identity to SEQ ID NO:25, wherein the sequence identity is at least 95% or 98% and the sequence identities are determined by analysis with a sequence comparison algorithm or by a visual inspection;
(b) having a sequence as set forth in SEQ ID NO:25; or
(c) encoded by a nucleic acid as set forth in claim 82.

84. An isolated or recombinant nucleic acid comprising a nucleic acid sequence
(a) having a sequence identity to SEQ ID NO:26, wherein the sequence identity is at least 90%, 95% or 98% and the sequence identities are determined by analysis with a sequence comparison algorithm or by a visual inspection;
(b) having a sequence as set forth in SEQ ID NO:26;
(c) that hybridizes under stringent conditions to a sequence comprising SEQ ID NO:26; or
(d) encoding a polypeptide as set forth in SEQ ID NO:27.

85. An isolated or recombinant polypeptide comprising a sequence
(a) having a sequence identity to SEQ ID NO:27, wherein the sequence identity is at least 90%, 95% or 98% and the sequence identities are determined by analysis with a sequence comparison algorithm or by a visual inspection;
(b) having a sequence as set forth in SEQ ID NO:27; or
(c) encoded by a nucleic acid as set forth in claim 84.

86. An isolated or recombinant nucleic acid comprising a nucleic acid sequence
(a) having a sequence identity to SEQ ID NO:28, wherein the sequence identity is at least 85%, 90%, 95% or 98% and the sequence identities are determined by analysis with a sequence comparison algorithm or by a visual inspection;
(b) having a sequence as set forth in SEQ ID NO:28;
(c) that hybridizes under stringent conditions to a sequence comprising SEQ ID NO:28; or
(d) encoding a polypeptide as set forth in SEQ ID NO:29.

87. An isolated or recombinant polypeptide comprising a sequence
(a) having a sequence identity to SEQ ID NO:29, wherein the sequence identity is at least 85%, 90%, 95% or 98% and the sequence identities are determined by analysis with a sequence comparison algorithm or by a visual inspection;
(b) having a sequence as set forth in SEQ ID NO:29; or
(c) encoded by a nucleic acid as set forth in claim 86.

88. An isolated or recombinant nucleic acid comprising a nucleic acid sequence
(a) having a sequence identity to SEQ ID NO:30, wherein the sequence identity is at least 85%, 90%, 95% or 98% and the sequence identities are determined by analysis with a sequence comparison algorithm or by a visual inspection;
(b) having a sequence as set forth in SEQ ID NO:30;
(c) that hybridizes under stringent conditions to a sequence comprising SEQ ID NO:30; or
(d) encoding a polypeptide as set forth in SEQ ID NO:31.

89. An isolated or recombinant polypeptide comprising a sequence
(a) having a sequence identity to SEQ ID NO:31, wherein the sequence identity is at least 85%, 90%, 95% or 98% and the sequence identities are determined by analysis with a sequence comparison algorithm or by a visual inspection;
(b) having a sequence as set forth in SEQ ID NO:31; or
(c) encoded by a nucleic acid as set forth in claim 88.

90. An isolated or recombinant nucleic acid comprising a nucleic acid sequence
(a) having a sequence identity to SEQ ID NO:32, wherein the sequence identity is at least 90%, 95% or 98% and the sequence identities are determined by analysis with a sequence comparison algorithm or by a visual inspection;
(b) having a sequence as set forth in SEQ ID NO:32;
(c) that hybridizes under stringent conditions to a sequence comprising SEQ ID NO:32; or
(d) encoding a polypeptide as set forth in SEQ ID NO:33.

91. An isolated or recombinant polypeptide comprising a sequence
(a) having a sequence identity to SEQ ID NO:33, wherein the sequence identity is at least 90%, 95% or 98% and the sequence identities are determined by analysis with a sequence comparison algorithm or by a visual inspection;
(b) having a sequence as set forth in SEQ ID NO:33; or
(c) encoded by a nucleic acid as set forth in claim 90.

92. An isolated or recombinant nucleic acid comprising a nucleic acid sequence
(a) having a sequence identity to SEQ ID NO:34, wherein the sequence identity is at least 85%, 90%, 95% or 98% and the sequence identities are determined by analysis with a sequence comparison algorithm or by a visual inspection;
(b) having a sequence as set forth in SEQ ID NO:34;
(c) that hybridizes under stringent conditions to a sequence comprising SEQ ID NO:34; or
(d) encoding a polypeptide as set forth in SEQ ID NO:35.

93. An isolated or recombinant polypeptide comprising a sequence
(a) having a sequence identity to SEQ ID NO:35, wherein the sequence identity is at least 85%, 90%, 95% or 98% and the sequence identities are determined by analysis with a sequence comparison algorithm or by a visual inspection;
(b) having a sequence as set forth in SEQ ID NO:35; or
(c) encoded by a nucleic acid as set forth in claim 92.

94. An isolated or recombinant nucleic acid comprising a nucleic acid sequence
(a) having a sequence identity to SEQ ID NO:36, wherein the sequence identity is at least 70%, 75%, 80%, 85%, 90%, 95% or 98% and the sequence identities are determined by analysis with a sequence comparison algorithm or by a visual inspection;
(b) having a sequence as set forth in SEQ ID NO:36;
(c) that hybridizes under stringent conditions to a sequence comprising SEQ ID NO:36; or
(d) encoding a polypeptide as set forth in SEQ ID NO:37.

95. An isolated or recombinant polypeptide comprising a sequence
(a) having a sequence identity to SEQ ID NO:37, wherein the sequence identity is at least 70%, 75%, 80%, 85%, 90%, 95% or 98% and the sequence identities are determined by analysis with a sequence comparison algorithm or by a visual inspection;
(b) having a sequence as set forth in SEQ ID NO:37; or
(c) encoded by a nucleic acid as set forth in claim 94.

96. An isolated or recombinant nucleic acid comprising a nucleic acid sequence
(a) having a sequence identity to SEQ ID NO:38, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:38;
(c) that hybridizes under stringent conditions to a sequence comprising SEQ ID NO:38; or
(d) encoding a polypeptide as set forth in SEQ ID NO:39.

97. An isolated or recombinant polypeptide comprising a sequence
(a) having a sequence identity to SEQ ID NO:39, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:39; or
(c) encoded by a nucleic acid as set forth in claim 96.

98. An isolated or recombinant nucleic acid comprising a nucleic acid sequence
(a) having a sequence identity to SEQ ID NO:40, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:40;
(c) that hybridizes under stringent conditions to a sequence comprising SEQ ID NO:40; or
(d) encoding a polypeptide as set forth in SEQ ID NO:41.

99. An isolated or recombinant polypeptide comprising a sequence
(a) having a sequence identity to SEQ ID NO:41, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:41; or
(c) encoded by a nucleic acid as set forth in claim 98.

100. An isolated or recombinant nucleic acid comprising a nucleic acid sequence
(a) having a sequence identity to SEQ ID NO:42, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:42;
(c) that hybridizes under stringent conditions to a sequence comprising SEQ ID NO:42; or
(d) encoding a polypeptide as set forth in SEQ ID NO:43.

101. An isolated or recombinant polypeptide comprising a sequence
(a) having a sequence identity to SEQ ID NO:43, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:43; or
(c) encoded by a nucleic acid as set forth in claim 100.

102. An isolated or recombinant nucleic acid comprising a nucleic acid sequence
(a) having a sequence identity to SEQ ID NO:44, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:44;
(c) that hybridizes under stringent conditions to a sequence comprising SEQ ID NO:44; or
(d) encoding a polypeptide as set forth in SEQ ID NO:45.

103. An isolated or recombinant polypeptide comprising a sequence
(a) having a sequence identity to SEQ ID NO:45, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:45; or
(c) encoded by a nucleic acid as set forth in claim 102.

104. An isolated or recombinant nucleic acid comprising a nucleic acid sequence
(a) having a sequence identity to SEQ ID NO:46, wherein the sequence identity is at least 90%, 95% or 98% and the sequence identities are determined by analysis with a sequence comparison algorithm or by a visual inspection;
(b) having a sequence as set forth in SEQ ID NO:46;
(c) that hybridizes under stringent conditions to a sequence comprising SEQ ID NO:46; or
(d) encoding a polypeptide as set forth in SEQ ID NO:47.

105. An isolated or recombinant polypeptide comprising a sequence
(a) having a sequence identity to SEQ ID NO:47, wherein the sequence identity is at least 90%, 95% or 98% and the sequence identities are determined by analysis with a sequence comparison algorithm or by a visual inspection;
(b) having a sequence as set forth in SEQ ID NO:47; or
(c) encoded by a nucleic acid as set forth in claim 104.

106. An isolated or recombinant nucleic acid comprising a nucleic acid sequence
(a) having a sequence identity to SEQ ID NO:48, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:48;
(c) that hybridizes under stringent conditions to a sequence comprising SEQ ID NO:48; or
(d) encoding a polypeptide as set forth in SEQ ID NO:49.

107. An isolated or recombinant polypeptide comprising a sequence
(a) having a sequence identity to SEQ ID NO:49, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:49; or
(c) encoded by a nucleic acid as set forth in claim 106.

108. An isolated or recombinant nucleic acid comprising a nucleic acid sequence
(a) having a sequence identity to SEQ ID NO:50, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:50;
(c) that hybridizes under stringent conditions to a sequence comprising SEQ ID NO:50; or
(d) encoding a polypeptide as set forth in SEQ ID NO:51.

109. An isolated or recombinant polypeptide comprising a sequence
(a) having a sequence identity to SEQ ID NO:51, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:51; or
(c) encoded by a nucleic acid as set forth in claim 108.

110. An isolated or recombinant nucleic acid comprising a nucleic acid sequence
(a) having a sequence identity to SEQ ID NO:52, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:52;
(c) that hybridizes under stringent conditions to a sequence comprising SEQ ID NO:52; or
(d) encoding a polypeptide as set forth in SEQ ID NO:53.

111. An isolated or recombinant polypeptide comprising a sequence
(a) having a sequence identity to SEQ ID NO:53, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:53; or
(c) encoded by a nucleic acid as set forth in claim 110.

112. An isolated or recombinant nucleic acid comprising a nucleic acid sequence
(a) having a sequence identity to SEQ ID NO:54, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:54;
(c) that hybridizes under stringent conditions to a sequence comprising SEQ ID NO:54; or
(d) encoding a polypeptide as set forth in SEQ ID NO:55.

113. An isolated or recombinant polypeptide comprising a sequence
(a) having a sequence identity to SEQ ID NO:55, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:55; or
(c) encoded by a nucleic acid as set forth in claim 112.

114. An isolated or recombinant nucleic acid comprising a nucleic acid sequence
(a) having a sequence identity to SEQ ID NO:56, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:56;
(c) that hybridizes under stringent conditions to a sequence comprising SEQ ID NO:56; or
(d) encoding a polypeptide as set forth in SEQ ID NO:57.

115. An isolated or recombinant polypeptide comprising a sequence
(a) having a sequence identity to SEQ ID NO:57, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:57; or
(c) encoded by a nucleic acid as set forth in claim 114.

116. An isolated or recombinant nucleic acid comprising a nucleic acid sequence
(a) having a sequence identity to SEQ ID NO:58, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:58;
(c) that hybridizes under stringent conditions to a sequence comprising SEQ ID NO:58; or
(d) encoding a polypeptide as set forth in SEQ ID NO:59.

117. An isolated or recombinant polypeptide comprising a sequence
(a) having a sequence identity to SEQ ID NO:59, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:59; or
(c) encoded by a nucleic acid as set forth in claim 116.

118. An isolated or recombinant nucleic acid comprising a nucleic acid sequence
(a) having a sequence identity to SEQ ID NO:60, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:60;
(c) that hybridizes under stringent conditions to a sequence comprising SEQ ID NO:60; or
(d) encoding a polypeptide as set forth in SEQ ID NO:61.

119. An isolated or recombinant polypeptide comprising a sequence
(a) having a sequence identity to SEQ ID NO:61, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:61; or
(c) encoded by a nucleic acid as set forth in claim 118.

120. An isolated or recombinant nucleic acid comprising a nucleic acid sequence
(a) having a sequence identity to SEQ ID NO:62, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:62;
(c) that hybridizes under stringent conditions to a sequence comprising SEQ ID NO:62; or
(d) encoding a polypeptide as set forth in SEQ ID NO:63.

121. An isolated or recombinant polypeptide comprising a sequence
(a) having a sequence identity to SEQ ID NO:63, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:63; or
(c) encoded by a nucleic acid as set forth in claim 120.

122. An isolated or recombinant nucleic acid comprising a nucleic acid sequence
(a) having a sequence identity to SEQ ID NO:64, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:64;
(c) that hybridizes under stringent conditions to a sequence comprising SEQ ID NO:64; or
(d) encoding a polypeptide as set forth in SEQ ID NO:65.

123. An isolated or recombinant polypeptide comprising a sequence
(a) having a sequence identity to SEQ ID NO:65, wherein the sequence identity is at least 65%, 70%, 75%, 80%, 85%, 90%, 95% or 98% and the sequence identities are determined by analysis with a sequence comparison algorithm or by a visual inspection;
(b) having a sequence as set forth in SEQ ID NO:65; or
(c) encoded by a nucleic acid as set forth in claim 122.

124. A polyketide comprising a glycosylated kinamycin made by a process comprising the following steps:
(a) providing a plurality of nucleic acid coding sequences, wherein the nucleic acid coding sequences have at least 95% sequence identity to SEQ ID NO:2, SEQ ID NO: 4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO: 26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO: 48, SEQ ID NO:50, SEQ ID NO:52, SEQ ID NO:54, SEQ ID NO:56, SEQ ID NO:58, SEQ ID NO:60, SEQ ID NO:62 and SEQ ID NO:64, wherein the plurality of nucleic acid coding sequences, when expressed in a Streptococcus, results in the synthesis of a kinamycin molecule in the Streptococcus;
(b) providing (i) a Streptococcus sp. or a Dactylosporangium sp. bacillus, or (ii) an intracellular extract of a Streptococcus sp. or a Dactylosporangium sp.; and
(c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated polyketide comprising a glycosylated kinamycin.

125. A method for making a glycosylated kinamycin comprising the following steps:
(a) providing a plurality of nucleic acid coding sequences, wherein the nucleic acid coding sequences have at least 95% sequence identity to SEQ ID NO:2, SEQ ID NO: 4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO: 26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO: 48, SEQ ID NO:50, SEQ ID NO:52, SEQ ID NO:54, SEQ ID NO:56, SEQ ID NO:58, SEQ ID NO:60, SEQ ID NO:62 and SEQ ID NO:64, wherein the plurality of nucleic acid coding sequences, when expressed in a Streptococcus, results in the synthesis of a kinamycin molecule in the Streptococcus;
(b) providing (i) a Streptococcus sp. or a Dactylosporangium sp. bacillus, or (ii) an intracellular extract of a Streptococcus sp. or a Dactylosporangium sp.; and
(c) inserting the nucleic acid into the bacillus of step (b) or contacting the nucleic acid with the intracellular extract of step (b) under conditions wherein the nucleic acid is transcribed into transcription products and the transcription products are translated into polypeptides, thereby making a glycosylated kinamycin.

126. A polyketide comprising a glycosylated kinamycin made by a process comprising the following steps:
(a) providing a plurality of polypeptides, wherein the polypeptide sequences have at least 95% sequence identity to SEQ ID NO:3, SEQ ID NO:5, SEQ ID NO:7, SEQ ID NO: 9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO: 31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO: 53, SEQ ID NO:55, SEQ ID NO:57, SEQ ID NO:59, SEQ ID NO:61, SEQ ID NO:63 and SEQ ID NO:65, wherein the plurality of polypeptides, when expressed in a Streptococcus, results in the synthesis of a kinamycin molecule in the Streptococcus;
(b) providing (i) a Streptococcus sp. or a Dactylosporangium sp. bacillus, or (ii) an intracellular extract of a Streptococcus sp. or a Dactylosporangium sp.; and
(c) inserting the polypeptides of step (a) into the bacillus of step (b) or contacting the polypeptides of step (a) with the intracellular extract of step (b) under conditions allowing synthesis of a glycosylated kinamycin.

127. A method for making a glycosylated kinamycin comprising the following steps:
(a) providing a plurality of polypeptides, wherein the polypeptide sequences have at least 95% sequence identity to SEQ ID NO:3, SEQ ID NO:5, SEQ ID NO:7, SEQ ID NO: 9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO: 31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO: 53, SEQ ID NO:55, SEQ ID NO:57, SEQ ID NO:59, SEQ ID NO:61, SEQ ID NO:63 and SEQ ID NO:65, wherein the plurality of polypeptides, when expressed in a Streptococcus, results in the synthesis of a kinamycin molecule in the Streptococcus;
(b) providing (i) a Streptococcus sp. or a Dactylosporangium sp. bacillus, or (ii) an intracellular extract of a Streptococcus sp. or a Dactylosporangium sp.; and
(c) inserting the polypeptides of step (a) into the bacillus of step (b) or contacting the polypeptides of step (a) with the intracellular extract of step (b) under conditions allowing synthesis of a glycosylated kinamycin.

128. An isolated or recombinant antibody capable of specifically binding to a polypeptide, wherein the polypeptide has a sequence selected from the group consisting of SEQ ID NO:3, SEQ ID NO:5, SEQ ID NO:7, SEQ ID NO:9, SEQ ID NO:11, SEQ ID NO: 13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO: 35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO: 57, SEQ ID NO:59, SEQ ID NO:61, SEQ ID NO:63 and SEQ ID NO:65.


SEQUENCE LISTING (65 sequences)

SEQ ID NO: 1 - DNA, 36321 bp, Streptomyces murayamaensis ATCC 21414
  misc_feature (1)...( 36321): n = A,T,C or G
cggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcccgggcggccgcatacgatttaggtgacactataggatcaactcggccaaccccgaggagtgggacgccatttacggggaggtcgagcgcggcgagaccgatgttctcctggtgagtccggaacgtctcaattccgtggacttccgcgaccaggtgctgcccaagctcgcggccaccaccggtctgctggtggtggacgaggcgcactgcatctccgactgggggcacgacttccggccggactaccggcggttgcgggcgatgctgaccgagctgcccgccggggtgccggtgctggccaccaccgcgaccgcgaacgcccgggtcaccgccgacgtggccgaacagctgggtaccggggccggtgaggcgctggtgctgcgcggcccgctggagcgcgagagcctgcgcctgggcgtggtccggctgccggacgccccacaccgcctggcctggctcgccgagcacctcgacgagcttcagggctccgggatcatctacaccctcaccgtggccgcggccgaggaggccaccgccttcctgcgccagcgcggcttcaaggtgtcctcgtacacggggcgcacggagaacgccgaccgtctgcaggcggagaccgacctccaggagaaccaggtcaaggcgctggtggcgacctccgcgctcggcatgggcttcgacaagccggacctcggcttcgtgatccacctcggctcgccgtcctcgccgatcgcctactaccagcaggtcgggcgcgccgggcgcggggtcgagcacgccgacgtcctgctgctgccgggcaaggaggacgaagccatctggcgctacttcgccgacacggccttcccgcccgaggtccaggtccgccagaccctgtcggccctcgccgacgcgggacggccgctgtccgtaccggccctggaggcggcggtcgacctccggcgcagcaggctggagacgatgctgaaggtgctggacgtcgacggcgccgtgaagcgggtgaagggcggctggacggccacgggggcggactgggtgtacgacgccgagcgctacgcctgggtggcccggcagcgggcggccgagcagcaggccatgcgcgattacgtgagcacgacgcggtgccggatggagttcctgcgccggcagctggacgacgagggggcggccccgtgcggccgctgcgacaactgcgcgggagcgtgggccgattccgccgtgtcggcggagacggtgacgggggcggcgaaggaactggaccgcccgggggtggaggtcgagccgcgccggatgtggccgacggggatggccgcgctgggcgtcgacctcaaggggcgcatcccggccaaggagcagtgctccaccggacgcgccctggggcgcctgtcggacatcggctggggcaaccggctgcgcccgctgctggccgagaacgcgccggacggacccgtcccggacgacgtcctgcgggccgcggtcgcggtcctcgccgactgggcgcgctcgccgggcggctgggcgcccgacgtcccggacgccgtcgcccgtccggtgggagtcgtcgcgatgccgtccctggcccgcccccgactggtcgcctccctcgccgaggggatcgcgacggtcggccgcctgcccttcctgggcaccctgacgtacacgggcccggacgacgcgcacgcggcgcggcgcagcaactccgcgcaacgcctgcggacgctgtcgggtgccttcaccgtctccgaggacctggccaccgcgctggccgccgcccccggccccgtcctgctggtggacgactacaccgactccggctggaccctggcggtagccgcccgcctactgcgccgcgcgggcagcgaccaggtcctccccctggtcctggcggctacgggctgagcttggccgctggggcgggaccgtcgtctaggatcacgggaactcggcccgcaggcgggccacttagcagaggatcaccggtgccgagccacatacgctccttctgggtcacctccccgggacacggtgagatcgtgacctcggcccgcctgcccgcctgcccgcctgcccgcctgcccgcctgcccgcctgctaatgatgaggccgtagtccggacgctgtattccggcgtcagccgtggcacggaatcgctcgttttccacggccgggtacccgtcagtcagtacgccgtcatgcgtgccccctttcaggaaggcgactttcccggacccgtcaagtacgggtatctcaatgtcggccgggtggaggaggggccggcggagctcgtgggccgggtcgtgttcagtctgtatccgcaccagacccgttaccgcgtcgcggccgacgccctcgcggtggtgcccgacggcgtgccgccggagcgtgccgtgctggccgggaccgtcgagaccgcgctcaacgcgctgtgggacgcgcccccgcggatcggtgaccggatcgcggtggtgggcgcgggcatggtcggctcctgtgtggccgcgctcctggctcggttccccggtgtccgcgtgcagctcgtcgacgtcgaaccggcccgccaggcggtcgccgcgcggctcggcgtcgccttcgccccgcccgagcgggccactgacgactgcgatctcgtcttccacgccagcgccaccgaggcggggctgaggcgctccctcgaactgctcgctcccgagggctgtgtggtcgatctgagctggtacggcgaccggccggtgacgctgccgctgggggagttcttccactcccggcggctgtcgctgcgcggcagccaggtcggcgccatcgcgccggagcgccgcgcacgccacacgagggccgatcggctcggcatggcgctcgacctcctgcgggacccggtcttcgacgtactgatcaccggcgagtccgatttcgacgaccttccgcaggtcatggccgagatcgccaccggcgcccggcccgggctctgccaccgcatccgctacggccccggctgaccgccgcagcacagccgcccgccgccaccatcctttcggagaagccatgttcagcgtcaccgtccgcgatcacctcatgatcgcccacagcttcagcggcgaggtcttcggccccgcccagcgcctgcacggagcgacatacctggtcgacgcgaccttccagcgaccggagctggacgacgacaacatcgtcatcgacatgggcctggcaggcagggaagtgcgcgggatcgtcgcggcgctctcgtaccgcaacctcgacgaggaccccgacttcgccggaaccaacaccaccacggaattcctggccaaggtcatcgccgaccgcctcgccgcccggatacacgacggcgccctcggccccggcgcccagggcctgaccggactcaccgtacggctccatgaatcgcacatcgcgtgggccgactaccaccgcacgctctgaccgcccggtcatggccatacgcccgtacgtcgtgctgtccgcggccgtctccctggacggccggctcgacgacacctcgcgcgaccggctcgtactctccaaccggcgcgacctcgaccgtgtcgacgatgaacgcgccgccgcggacgccatcctggtcggcgccaccaccctgcgcagggacaacccgcgcctgctggtggcgagcgccgatcggcgggcccggcgcgtcgccctcggcatgccggagcatccgctgaaggtcacggtcaccgggtccgccgaggtgaacacgtcgtacgcgttctggcattgcggcggcgagaagctggtgttcacggtggacggagcgctcctgcgggcccgtcgcaccgtaggcgacctcgccgacgtcgtcagcaccggccccgctctcgactggcacctcctcctcgacgaactcgggcgacgtggggtggaacgccttctggtcgaaggcggcgggacggtgcacacccagctgctggcccaggacctggccgatgaactccacctcgtcgtcgccccgttgctggtgggcgaagccggtgcgccggtgttcctgggcccggcgtcgtacccgggtggtcccgcggcccgtatgacgctgctcgaagcacgccccgtcggtgatgtcgtgctgctgcgctacgccccgaaactccgatcctgagacggtgagcccgcacaagtcggccagtgccgagccgagttgacgttccgtcggtatgctcctcgggcgcgtggccggacggtgtgcggacgagacgggggcgagtgtggacgacgcggtggaactggcggagatgatcggccagttgcggagcgagctgagccgggccatggcggacggggccgcgggcggcgggctgcggttccaggcggagaagctggagctcgaactcaccgtgggcgtggagcgcagccgggagccgggtgcgaaggtgcggttctgggtgctcgacgtccacggctccgcccgctccgcgcggaccgccacgcagcggatcaagctcaccctgcaaccggtgctcggcgacgcacccgacagccccgcgctgatctcgggcgcggagctgcccgatgagagctgaaccgtcggccgacgggctcgacccgcaccggatcgccgagatcatcgtcgagagccccgagggcagaaggcgcggttccggctaccggatctccaccacgacggtcctcaccgccgcccatgtggtcgccgacgcgacccgcacgctcgtacggtgcgacgccgaccagcccgcggagtggtcggctccggccatggtgacctgggcggatgcgggcagcgacctcgccgtgctgagcgtcggggcacctgcggccgcccccgtggtcaccgcacccgcccgcttcgcgcgcatcgccgacgaccggcacggggtgatcggggtgcacgccgccgggtttccgctgtggaaacgccgacgccggtccgacggcgcgtacttccgcgaactgcaccaggcggacggcacggtggcggcgctctccaatctgcggaccggcacgctggagatgaccgtggcaccggccggaaccgaccccgacccggcggcctcgccgtgggcgggcatgtcgggcgcggcggtgtgggccggcagccgcatcatcggcgtggtcgccgagcaccaccgctacgagggccccggacggctcaccgcggtccgcctcgaccacgcgctgcgcgggctcggcgccgcgagacgggcggagctggcccggttgctcgcgctgcccgagaccgcggatctgcccctcgccgtccccgggcaaccgcacgacgaccgcgccccgggggtgcgggtggtcggcgtccccgtcgcgcacggcatcgagctgttcaagaaccgcacccgcgaaagcgacctgatcgcccgtcacttgagcgatccggccatccgtatggtgtccgtcgtcggacggcgcggcatcggcaagagcgcgctcgccgcgaaggtcatggatctgctggaccggggggaatggcccggcgcggccgccggtcccctcccgtccggcctcgtcaacctctccacccgaacctccggcatctccctggagcggctctacttcgactgcgtccggctgctcggccccgagcacgaggagcggctgcgcggggtctgggcgggcggcggcagcgcgcaggaccgcctcgccgcactcttcgacgccatgggcgggcggctgatcgtcatcctcatggacaacttggaggaactgctcggcgacgacggcggcatcgaggacgaggagctggccctcttcctggactggctgttccgggcccgcaccaccccacgtctgctcgtcaccagccaggtgccggtgcgtctcgcgcccgaactgcgccgcttcgccgccgaggtgccgctctccgaggggctgcgccccaccgaggccgcggccctgctgcgcgaactcgaccgggacggcagcctcggcatcgccgacctgtccgacggcgaactcctcgacgccgcggtccgggtgcacggcgtcccgcgcgcgctcgaactcctcgtcggcgcggtcgccgaggagacggtgctgctgcccagcctgaaagacgtactggaggacttcacccgccgccacgacgtcgtcgcggacctcgcccaggaccgctaccgtcgcctcgacgagccggcgcgtgccgtcctcggtgtgctcgccgccctgcgcacccccgtggagcagggcgcggtggcgcagatcgcgggcgggctcgatccggacctgcgggtggtccccgtcctcacggcgctcgtccgggtccgcctggtctccgtggaccgggccagccggacggtcgccctgcacccgttggacgcggacatcgcccgtgaccagatgccgtgggacggacctttcgggaggcaagcggtggaacggcagatcgcagcctggtacgcgcggcgggcgaagccgcgcggcgcctggcggacgctggaggacgtcgagccgcagcggcggcagttcgaccacctggtcggcgcgggcgaccacgacgcggccgcgcgggtgctggccgagatcagcgaatggctcgtctggcacggctcggtgctcgccgcggtcacgatgcacctggcggtcgacggacacctccgcgacgagcgggtacgcctcgcccacaccgtcgcgtacgggcacgcccggctcagtgcgggtcccatggagcaggcggtcgagctgttcaccgaggccgcggccctcgccgaacgcctcggcgaccggcccgcgctgcagaacgcgctgttcggcctgggcgacgcccaccggcagctgggcgatcagggcgccaccgtcgaaccgctcgcccgcgccgccgagttggcgcgtgaactgggcgacaccgagcgcgaggcgcacgcgctgctctccctcagcctcgcgcacagctacctcggcgacggcgagcgcgcgctggagggggccgaccggctggccgcgctcgccgaggcgggcggcgacccgctcgcgctcgcccgcgccggcaacgcgcgcaccatcgcgctgctcaccctgggccgctggcaggagaccgccgaggcgggcgccgagacggcccgcgcctatcgcgccgccggcagtcaggaagcggtcgcgtacgcgctgaacgcgcagggcctggccctgctggccctggacgacccggcgcgggcggccgcggtgctcgaagaggcccgccacgaggcctcactgatggagagcccccgggccgagggcgtctgcctgctcaacctgtcctgggcgtactggtgcgacggccgccgggacgagtgcgcggccaccgccgaacgcgcctcgaccgccctccagatcgccggagcgacccaggcggcggcggcccgttcgctggccgaggccgcccgcgtcctgcccggcgaccctggggccgccgccgacgccctgatccgcgcggcggccgcgctggacggcaacgccgaggtcatcgcccccgcccggctcaccgccgaggcacgccgactgctggactgacccgctggggtgcgccgctaggacgtggcgcggtcggagcgcaatccgtcgagcacgatgtcgatgtagcgccgccagtcgccgctgcgccggtgcacgatcgaggtgagaccgcaggtcagcgcgaggatgtcggcgcccgcgatatcggtgcggatggagccggccgcctggcccttgccgaccaggtccaccagctcgtcctccaggtcggcccgcatggtgctgggcggcccctcggagccgagcgtcccgccgacgacgctggcgaagccgcggtcccgggcctccacctcgccgacccgggtgagcagcatctggagtgcttccagcggctcggccgactcccggcagaccgtgcggtagtacgtgaggatctcgccgaagcgctgctgggaggccgcgacgaccatggcttccttggtggggaagtggcggtagagcgtccccacgccgacctcggcgcggcgggccacctcgtccatggacacgttggcgccgcgttccgcgaagagctccctggccgcgttcagcacccgggcccggttgcgctccgcgtcggcccggagccggcggggaccgttctcggtgactggcgttgacgtaggcatacggaacctccctccgtttgactctactatgccctgccgttacctactgaaccagccctgacttcgcgccagttggggtatgcggttcgcagttgcccagtcggttacgcggtgcggcccggggtgttgacaatggcctggtcgtacagtgaaacggaaggacgaacacgatgtcgttgcccctggggcgtacgagtgcgccggaggtgtcggcgtcggtggaggcattcctgacccagacgccgcacatcggcgtgctgaccaccatccggcccgacgggtccccgcatgtggcgccggtgcggttcacctgggacgcggaggcgggtctcgcccgggtgatgacggtgtcctcgtcccgcaaggcgcgcaatctgatcgccgcgcccggcagccgggtcgccatatgccaggtggccgggttcgcctgggtcaccctcgaaggctccgccgtggtggccgacgacccggtgcgggtcaccgagggagcgcgccgctacacccgccgctaccgctccgggccgcccaacccgcccggccgggtggtcgtggagatctcggtcgaccgggtcatgagcctcaacgtctgaaccgggttccgcaccgagttccgcgcaaaggcggaacaagggcggaacaacggcagagcgcgnnnnnnnnnnnnnnnnnnnnnnnnnnnnnnnnnnnnnnnnnnnnnnnnnnnnnnnnnnnnnnnnnnnnnnnnctcttttcgtgctgtcccggcggtcgcccgcgtcagcccttgcggggcgcggtgccggtcacccagatcatgcgcagggacagcaggctgagccgccagccctccggggtgcggcgggcctcggcggtcaccaggcttccgttggcgaatatcggatcgcggtcggcgtcctccggatggtcggacgggtggtgcacatgggtggagacgatgttgcaccgcaaagaggcccggtcgccgtcgatttccacgacggcgggcgaaccgatgtgctgcgtgcgcgcgaaggccgcgagcgccgtgctgtggtactcggcgagtccggcgatcccctcgtgccggctcatcgggaactcgacgaccgcgtcctcggtgaacagcccccgggcccaggcgtcgtcgagcttgtcgtcgtccagattgatcaggtaacggtccagaagtcccgcgacatcggcggtggattggcttgaggtcatggttgaggatgctccggaacaagccgctgaccaagactccttgtagcagacctgacgtacttgtcagaggtctgtactagcggctatttacgcccgctggacccctcgggatactggtgaaacgccagtcggaagagggtagatggataactttgacgcggacgtaattatcgcgggagccggccccactggactcatgctcgcaggcgaactgcgcctgaacggcgtgtccgtgattgtcgtcgatcgccttgccgagccgatacagcagtcgcgtgcgctgggattttcggcccgtaccatcgaggaattcggccagcgcggactgctgtcccgtttcggtgaagtcgacgtcattccggtcggtcatttcggcggagtgtccatcgattaccgtctggtcgagggcggttcgtacggagcgcgcggcattccccagtcgcgcaccgagggcgtgctggcgggctgggccggacagctcggcgccgaggtgcggcgcggggtcgaggtcacgggcctggacaacggcgccgacggcgtgagcgtggaggtcagcaccgccgaggggcccgccacgctgcgcggccgctacctggtgggcgcggacggcgcccgcagtgcggtgcgcaagctcgcgggcatcgacttcccgggcaccgacccggccatcgagctccggttcgccgacatcagcggggtgccgttgcgcccgcgcttcagcggcgagcgggtgcccggcggcatggtcatggtgctgccgctcggcccggagcgctgccggatcgtctacttcgaccgcagcgagccgttgcgcaagagcccggacccgatcaccttcgacgaggtggccgaggcgttcaagcggctgtccggcgaggacatcagcggcgccaccgtgcactgggtctccaccaccaccgatgtgagccggcaggccgccgagtaccgcaggggccgggtcttcctggccggcgacgccgcccacatccatctgcccatcggcgcccagggaatgagcgccggcgtgcaggacgcggtcaacctcggctggaagctcgccctggagatcaagggccaggctcccgagggcctgctcgacacgtaccactccgagcggcacccggtcggcgcgcgggtcctcaccaacacgctcgcccagcggatcctctacctcggcggcgacgagatcaccccgctgctcgatgtgttcaccgagctcaccgggttcgaggacgtccagaagacgctgatcggcatggtcaccggcctggacatccgccatgacgtgggcgagggcgaccatccgctcctcggccgccgtctcaaggacgaggagctggtggtcgacggcaagaagaccaccaacttcgaactcctcgcggccggcaaggccgttctgttcaacctcaccgatgacccccacctgcgggagctcgccgcgggctgggccgaccgtgtcaccacggtcaccgccgagcagcacgactgcgacaacggtctggacgccttcctggtccgtcccgacggctatgtcgcctgggtcgccccttccgcgtcgcgcacggaggggctcgccgaagcactcaaccgatggttcggccggcccaactgagccgggcctgactccctttcacatccgtagacccgaaggaagcgaagaacatgcccaagatttcctctgatgacaagcacctgaccgtcctcaacctgttctccacggacgccccggagaagcaggagggtctgctcggcgcgatgcgcgagatcgtcgacgcggccgcctacccgggctggatgtcgtccaccgtgcacgccggcgtggacaagccgggcacggccaacttcatccagtggcgcagccgcgcggaccttgaggaccgctacgacggcgaggagttcaagcaccgcacgctccccctcttcggcgagctgaccacctcgatccggctgctccagaacgaggtcgcgtactcgcagaccaagtcgggcgacagcgtcgagatctccccggcccgcaccgacttcaccgtcatcgcggtcttcggtgtcgaggagaagaaccaggacgacctggtcgacgcgctcggcccgtcgatgaagttcctcagcgacgttcccggctacgtctcgcacaccgtcctgaagggcatcgcggcccgtggccttgagggctccttcgtggtctcctactcgcagtgggagagccaggaggccttcgtcgcctaccaggccgtcgcgcaggccgacaagcccgccgcccgccaggacgcggagaagcgcaccggctcgctcctgacgtcggtggactccaacacctaccgcgtggtccacacccgcgcggccggcgagtaacgagacggagcgttccgctccagcacgacacgtacgcacggacgcccgactcccctcgcggagccgggcgttccggcgttttggggcccggtcgcgcccctttccgccgccaagacggtccggttcgcgacctgacggcacctgacgcccacgtgacaattccgcgccgactcgctgctcttcgctgcgagttctctgacatggctgcgggacatttgcggaacctgaccgaaccggacgtagacctgaaccggggcgccttgaaaggacatgcccgctcagcggacccttgaatgcatgatccggggcgtggttcgccaccgctgatcctctcgctcaactcccgctcatggccctcgcattcgggcgcgtccgacgtcgtacgcgcaagaggcggagccagtgccggatcaaccgcaaccgctgaggagcgaatacgtatgcacagcacgctcatcgtcgcccgcatggaacccggttcgagcaccgacgtcgccaagctcttcgccgagttcgacgcgacggagatgccgcaccggatgggaacgctgcgccgccagctgttctcctaccggggcctctacttccacctgcaggacttcgacgcggacaacggcggtgagctgatcgaggccgcgaagaacgacccgcggttcatcgggatcagcaacgacctgaagccgttcatccaggcgtacgacccggccacctggcgctcgcccgccgacgccatggccacgcgcttctacaactgggaggggcgcgcgtgacaacgcctcgcagggtggtcatcaccgggatggaggtcctcgcccccggtggcatcggcaccaagaacttctggagcctcctcagcgagggccgcacggcgacccgggggatcacgttcttcgaccccacgccgttccgctcgcgggtggccgccgagatcgacttcgacccgtacgagcacggtctgagcccgcaggaggtccgccgcatggaccgggccggccagttcgcggtcgtcgcctcgcgcggcgcggtcgccgacagcggtctggagctcgccggcctcgacccgtaccgggtcggtgtcacggtcggcagcgcggtcggcgccaccatgggcctggacgaggagtaccgggtcgtcagcgacggcggccggctcgacctcgtggaccaccagtacgcggccccgcacctctacaaccacctggtgccgagctcgttcgcggccgaggtggcctgggcggtcggcgccgagggccccagcaccgtggtctccacgggctgcacctccggcatcgactcggtcggctacgccgtcgagctgatccgcgagggctccgccgacgtcatgatcgccggatcctcggacgcgccgatctcgccgatcaccatggcgtgcttcgacgcgatcaaggccacgacgaaccgttacgacgagcccgagacggcctcgcggccgttcgacaactcgcgcaacggcttcgtcctgggcgagggcaccgcgttcttcgtcctggaggagctggagagcgccgtcaagcgaggcgcccacatctacgcggagatcgccggctacgccacgcgctccaacgcgtaccacatgaccggactgcgccccgacggcgcggagatggccgaggcgatccgcgtggcgctggacgaggcgcggatgaacggcgacgagatcgactacatcaacgcccacggctccggcaccaagcagaacgaccgccacgagacggcggcggtcaagcggatcctcggtgaccacgcctaccggacgccgatgagctccatcaagtcgatggtggggcactcgctcggcgcgatcggctccatcgagatcgccgcgtccgcgctcgccatggagtacaacgtcgtaccgcccacggccaacctgcacacgcccgaccccgagtgcgacctggactacgtcccgttgaccgcccgcgaccgcaagaccgacgcggtcctctcggtcggcagcggcttcggtggattccagagtgccgtggtgctcgcccgtcccgagaggaagctcgcatgacgtcgtccgtggtggtcaccggcctgggggtggcgtcccccaacggactcggcatccaggactactgggcggcgaccgtcggtggcaagagcggcatcggccgtatcacccgcttcgacccgtcgtcctacccggccaagctggccggcgaggtcccgggcttcgtcgcggaggacctgctgcccagccgcctgctcccgcagaccgaccgggtcacccggctggcgctcgtcgccgccgactgggcgctcgccgacgcgggcatcaccccctccgaactcggcgagttcgacatgggcgtggtgaccgcgagcgcggccggcggcttcgagttcggccagggcgagctccaggcgctgtggtccaagggcagccagtacgtctcggcgtaccagtccttcgcctggttctacgcggtcaacagcggccagatctccatccgcaacgggatgaagggccccagcggcgtggtcgtcagcgaccaggccggcgggctcgacgcggtggcgcaggcccggcggcagatccgcaagggcaccagcgtgatcgtgtccggcgccatcgacgcctcggtctgcccgtggggctgggtggcgcagctggccagcgaccggctctccaccagcgacgagccgacccgcgcctatctgccgttcgaccgcgacgcctcgggctatgtggcgggcgagggcggcgcgatcctgatcatggaggacgccgagtcggcccgcgcccgtggcgcccgtgtctacggcgagatctccggctacggctcgaccatcgacccgaaggccggctccggccgcccgccggggctgcgcaaggccatcgaactcgccctggcggacgcgggggtcgccccgggtgaggtggacgtggtcttcgccgacgcggccgccgaccccgagctcgaccggcaggaggccgaggccatcaacgccgtgttcggcacccgcggcgtgccggtcaccgctcccaagacgatgaccggacggctctactcgggcgccgccccgctggacctggccgccgccttcctcgccatgaaggacggtctgatcccgccgaccgtgcacatcgatccggccgccgagtacgacctggatctggtcaccggcgagccgcgcaccgccgaggtgcgcaccgcgctggtcgtggcccgcggctacggcgggttcaactccgcggtggtcgtgcgcgccgcgtagcgctccgcgggagtgcgttgttcgactgcaggccgtctgtggctggtcgcgcagttccccgcgccccttcggggcgcggtacttacctaggaaagggaaatcccatggccaccacgttcaccctcgacgacctcaagcgcatcctccttgaggcagccggcgccgacgagggcgtcgacctggacggcgacattctggacaccgagttcgaggtcctgggatacgagtcgctcgccctgctggagaccggcggccgcatcgagcgcgagtacggcatctcgctggacgacgacgcgctgaccgacgcggtcaccccgcgcgccctcatcgaggtcgtcaacgcccagctgtccgccgcgtccgccgcctgagccgctcgaacaaggaagaggaatcatgaccgagaacaccgcacgggtcgcgctggtcacgggtgccacgagcggcatcgggctctccgtcgcccggctgctcggctcgcagggccacaaggtcttcatcggcgcgcgcaacgccgacaacgtcgccgagacggtcaagcagctccagggcgagggcctggaggccgacggctcggcgctcgacgtcaccgacgccgccagcgtcaaggccttcgtccaggcggccgtcgaccgcttcggcaccgtcgacgtgctggtgaacaacgccggccgctccggtggcggcgtcaccgccgacatcgaggacgagctgtgggacgccgtcatcgacaccaacctgaacagcgtcttccgggtcacccgtgaggtcctgaacaccggtggcatgcgccacaaggaccgcggccggatcatcaacatcgcctccaccgcgggcaagcagggcgtggtgctcggcgccccgtactcggcctccaagcacggtgtggtcggcttcaccaaggccctgggcaacgagctcgcgcccaccggcatcacggtcaacgccgtctgccccggctacgtcgagacgccgatggcgcagcgcgtgcgccagggctacgcggccgcgtactccacctccgaggacgcgatcctggagaagttccagtccaagatcccgctcggccgctactccaccccggacgaggtcgccggtctggtcggctacctcgcctcggacacggccgcgtccatcaccgcgcaggcgctcaacgtctgcggcggcctcggcaacttctagcccacgtacccgaaggagcagcggatatgacgacccgcgaggtcgagcacgagatcaccatcgaggcccccgccgccgccgtgtaccggctgctggcggaggtcaccaactggccgcggatcttcccgccgacgatctacgtcgaccaggtgggcgagcacgacaaccacgagcgcatccggatctgggccaccgccaacggcgaggccaagaactggacctcgcaccgtgagctcgaccccgaggcgctgcggatcaccttccgccaggaggtcaccacgccgccggtcgccgcgatgggcggcacctggatcatcgagaccctgggcgagaccacctcgcgggtccggctgctccacgactaccgggcgatcgacgacgaccccgaggggctggcctggatcgacgaggcggtcgacaagaacagccgctcggagctggccgcgctgaagcagaacgtcgaactggcccacgcgaccgaggaggtgacgttctcgttcaccgacaccgtcatcgtccagggctcgcccaaggacctgtacgacttcatcaacgaggcgaacctgtggtccgagcggctgccgcacgtggccgtcgtccggctcaccgaggacaccccggggctgcagaccctggagatggacacccgcgccaaggacggctcggtgcacaccaccaagtcgtaccgggtgaccttcccgcaccacaagatcgcgtacaagcaggtcacgctgcccgcgctgatgaccctgcacaccgggatctggacgttcgaggagacgcccgagggcacggccgcctcctcgcagcacaccgtcacgctcaacacggacaacatcgcgaagatcctcggccccgaggccaccgtcgcggacgcccgtgagtacgtgcacaccgcgctgtccaccaacagcacggcgacgctcaaccacgccaagacgtacgccgagtcgaagggctgagccacagatgaccccggaccgcctggacacacaggtcatcgtcgtcggcgccggccccgtcgggcttctgctcgccggtgagctgcgtcttggcggcgccgacgtggtcgtactggaacaacgggccacgcccaccacggagtcgagggcctccacgctgcacgcccgcaccatggagctccttgacagccgcggcctgctcgacctgttcgggacgccgccgaacgagccgcgcggccacttcggcggcatcccgatggacctcacgctgcccagccccttcccggggcagtggaagatgccccagacccggaccgaggcgctgctccaggagtgggcgctgtcgctgggcgcggacatccggcgcggccacgagctggtcgccgtgtccgacgagggcgacttcgtcgaggcccgggcggccgggccggagggcacggtcgtggtgcgcgggcggttcctcgtcgggtgcgacggcgaggagtcggccgtgcgccgcctgacgggcgccgagttcccgggcaacgacgccggccgcgagctgctgcgcgcggacgtggccggtgtcaccatcccgggccgccgcttcgagcggctgcccgccgggctcgccatcgcggcgacccgcgacggggtgacccgggtgatggtgcacgagttcggctcccaggccgaaccccgcaccggcgacccggagttcggcgagatcgcggcggtctggaagcgcgtcaccggcgaggacatcagcggcggaaccccgctgtgggcgaactcgttcggcgacgccaaccgccagctcacgcactaccgcgacggccggatcctgttcgccggcgacgcggcccaccggcagatgccgatcggcggccaggccctcaacctgggcctccaggacgccttcaacctgggctggaagctggctctgcacctcggcgagtcggcccccgagggcctgctcgacacgtaccacagcgagcggcacgaggtcggccggcgggtgctttccaacatcagggcacaggccatgctgctgctcggcggccaggaggtcgagccgctgcgcgcggtgctgaccgagctcctgccgtacgacgacgtccgggcgcacctcgccgggatgatcagcggcctcgacatccgttacgacgtgggcggccccgagcacccgctgctcggcgcacggctgccggacgccggtctcaccaccggcgaaggcccgctgagcaccgcccagttgctgcgcaccgcacgcggtgtgctcctcgacctgtccggtggcagtgcggtgctgtcggacgccgccggctgggcggaccgggtcaccgctctgcccgccgtgccggagaagggcggcgccctcgactcggtgggcgccgtcctggtccggcccgacggccatgtggcctgggccggcgccccggacaccgacggcgccgggctgcgggaggccctggagcgctggttcggcccctcgcactgagctcccgtaccgcgagaaaccccccacccccgcgcacgaccgtacgactccacctcacagcacgacagggcacgtccaggaaagggaacactatggaggggacagccgtggacaccgatgtgatcatcgtcggcgcgggtccgaccggcctcatgctcgccggggaactgcgcctcggcggggcggacgtcgtcgtcgtcgaacggctgacgaagcccaccggccagtcccggggcctgggcttcaccgcccgcgccatggagatcttcgaccagcgcgggctgctgccccggttcggccagggcgagacgctggagatcagcccgctcggtcacttcggcggtgtgcagttcgactacaccgtcctggagggcgcccacttcggggcgcgcggcattccccagaacatcaccgagacggtccttgaggagtgggcgaccgagctcggcgtggacatccggcgcggctgggacttcctggagatagccgacggctacctcgacggcgacagcgtcgagatcaaggtgcagacgcccaactcggtacggaagctgcgcgcttcctacctcgtgggcgccgacggcggccgcagcgtggtgcgcgaggcggccgggttcgacttcccgggcacctcggccacccgggcgatgttcctggccgatgtgaccggctgcaacctcaagccgcgcttcctcggtgagcggctgaacaacggcatggtgatggcggccccgctcgccgagggcgtcgaccgcatcatcgtctgcccggacggcacgcccgcgcgcgccagcggcgacacggtcagcttcgaggaggtcgccgccgcctggcagtcgatcaccggcgaggacatctcgcacggcggcgccgagtgggtcagcttcttcagcgacgccacccgccaggcctccgagtaccggcgcggccgggtcctgctggtcggcgacgccgcccacatccacctcccggccggcggccagggcctgagcaccggcgtccaggacgcggccaacctcggctggaagctggccgcggcggtcgccgggaccgcgcccgaggggctgctcgacacgtaccacggcgagcgccaccccgtgggtgcccggctgctgatgaacacccgcgcccagggcatggtgttcctcggcggacccgaggccgagccgctgcgccagctcttcggcgagctcatccagtacgacgacgtgaagcgccatctcgccgggatcgtcagtggtctggacatccggtacgagctgggtgacgcgcacccgctggtggggcgccggattccgcctcggcggctggtgggggcggcgggggagaccagcaccgtcgcgctgctgcacgcggcgcggggtgtgctgctcgacttcgccgacgacgcggcggtgcgggacgcggccgccgggtggtcggggcgcgtcgacgtcgtcacggcggcgccgaagccggtcgacggcggtaccgatccgctcgcgggtgcggctgccgtgctcgtacggcccgatggatatgtggcgtgggccgcggacacggccgaaggccttgctccggctcttgagcgctggttcggtccggccggggtgtgacgcccactcactcggtcccgaaggcggtttcgcgtctgcgagccgtcccgggttgctcgcgcccacgcggcgaagccgcacatgtcacagccccgcgccccttcgggcgcggggcaggcggcccactcaacagtcaacgcaaacgggggcattgatggaaagcacgctcgcaccgggcgcggtctcccagggcgttcgcaggatcaccctggacgccgggggagtcacgctgtccgcgctgctgtgcgagccggaagggaccccccgcgccaccgtcgtcgccgtgcacggcggcgggatgagcgccgggtacttcgacggtcaggcgcaccccgagctgtccctgctcaccctcggcgcccggctcggctacaccgtgctcgcggtggaccggcccggctacggccgttccgccgcccagctgccggacgggctcaccgtcgccgagcagaccgaggtgctgcgggccgggatcgacgacttcacctccaagtacccgacgggcgcgggggtgttgctggtcgcccactccttcggcggcaagctcgccctgtcggccgccgcgcactgcaccggcgacggcctgctcggcatcgacatctccggctgcggccaccgctacgccgtcaccccgggcgtgctgcgcaagggcctcaagcacatcgcccggcactggggcccgctgcggctctacccgccggacaccttccgcagcagcggctccctggtggcgccgatgccggagcgcgaggcgagtgaactcaagcgctggcccgagctgttcgcggccctcgcgccgcgcgtgcggatcccggtccggctcaccttcgccgagcacgagggctggtggctgcacggcgagcaggacctcgccgacctcgccgcccagctgaccgcctcgccccgtatcgtcgtcgaccgccagccggacgccggtcacaacatcagcctcggctgggcggcccgctcctaccacctgcgcaccctcgcgttcctggaggactgcatcacgcgggcgggacgcgatgggtgatccgagcctatgaccagcactctggcaacccctttccgttccctgtccgtacggaacttccggctgttcgcggccgggcaggtggtctccgtcgcgggcacctggatgatggtcgtggcccaggactggatcgtcctgagcctggccgacaactccggtacggcgctgggcgtggtgaccgcgctgcagttcaccccgctgctgctgctcaccctgtacggcgggcgcctcgccgaccgctacgacaagcgcttcctgctgacctgtgccaatctcgcgtccggcgcgctggctctggtgctcgcgctgctcgcgttcgcggacgcggtgcagctgtggcacatctggctgtgcgcgttcggcctcgggatggtgaacgccgtcgaggtgccgacccggatggcgttcgtcagcgagctggtcggccccgaactgctgcccaacgcctccgcattgagcgccgcgtacttcaacaccgcccgggtcgtcggcccggcgctggccgggctgctcatcaccggcttcggcaccggctgggtcatgctgttcaactccgtcagctatctggccacggtggccgggctgcggatgatgcggccggacgaactgctgcgcggcgcacggcaggacacccgtccccgggtgatcgacgggctgcggtacatccgcagccgccccgatctgaagctgccgctcgccctgatcggggtgatctcgctcgtcgggctcaacttccagctgacgctgccgctgtatgccaaaacggttttccacgccgacgcggcctcgttcgggctgctgaccaccggcttcgcggcgggctccctggtcgccgcgttcgtcaccacggcgcgccgcggccgcccctccagccgtctggtggtcgcctcggcgatcgcgttcgcggccttggagacggtggcgggctgggcgcccaacttcgcctcggcgatcgtgctgctctcgctcaccggcggggcgaccatctacttcgtccaggcggccaaccatcgcgtccagctcggcagcgacccgcagtaccggggccgggtgatggcgctctacacgctcatcgtccagggctccaccccgctgggatcgctcctcatcggctggctcgccgaacacctgggcgcccgctcggggttctacgtgggcggcctggtctcgctggcggccgccctgacggcgctggccttcgaccggcgtacgggacaggaggcgtccgacgacgtgacgacggcgacgaaggccgcgcccgagggcgagcccgaggcggtgagccggtgaacgcgaccgtgacaccggtgaacgccaccgtgacgccgctgaaggcgccgcccggggcgggcctgctgcatctggtcgtcttccggccgcccgtcgaggacgcggcgccggtgtgcccgctgatgcaggccctggacgagctggaggcggcccgcgcggccaccttcgtccacgacagggaccgccgccagtacgtggcggcgcacgccaccctgcggcgcgtgctcgccgagtacaccgggcacgagcccagccgggtgccgctcggccgggccgaagggccctacgggaagccgcagttgatcggttcgccggtcccgctgcacttcaacctctcgcacagccacggcctgatcgccatcggggtcgcggcggacccggtgggcgtcgacgtccagcgcgtcccgtcgcccgaggcggtcgaggtggtcctgcccaggctgcatccgcgcgagcgcgaggaactgcgcgctctgcccgcatcggagcgcccggaggcgttcgcgcggctgtggacccgcaaggaggcctacctcaagggcctgggcaccggcctcacccgctcgcccgcggcggactatctgggcgagacggccgcggcccgcccggccggctggacggtgcgcaacgtgccggtacagccgggctacgcggccgcggccgcgctccgccaccacccgacctgatcgagaaggcagtcgattccgctccaggattccgcaaccacacagggggaaaacgaaccatgccgaccacgccgaccacacagtcctccgccgaggtctccgaccggctggacgaactcagcgaacgcaaggaacaggccgtacgcggtcccagcgacaaggcgaccgaggcgcagcacgccaagggcaagctgaccgcacgcgagcggatcgaactcctcctggacaagggcagcttcaccgaggtcgagcagctgcggcggcaccgcgccaccgggttcggcctggaggccaagaagccgtacacggacggtgtcatcaccggctggggcacggtcgagggccgtacggtcttcgtctacgcccatgacttccgcatcttcggcggcgcgctcggcgaggcccacgcgaccaagatccacaagatcatggacatggcgctggcggcgggcgcgccgctggtctcgctgaacgacggcgcgggcgcccggatccaggagggcgtctcggcgctcgccggttacggcggcatcttccagcgcaacacgcgggcctcgggtgtcatcccgcagatctccgtgatgctcggcccgtgcgcgggcggcgcggcgtactcgccggcgctgaccgacttcgtgttcatggtccgcgagacctcgcagatgttcatcaccggcccggacgtcgtccaggccgtgacgggcgaggagatcagccagaacggactcggcggcgccgatgtgcacgccgggacctcgggcgtggcgcacttcgcgtacgacgacgaggagagctgcctcgccgaggtgcgctatctgctctccctgctgccgtccaacaaccgggagatgccgccgctggcgcagacctcggacccggtggaccgcgagggcaccgccctgctcgatctggtgccggccgacggcaaccgctcgtacgacgtgcgcggggtgatcgaggagctcgtcgacgacggcgagtacatggagatccacgccaactgggcgcccaacctggtggtggccctggcccggctggacggccatgtcgtcggcgtcgtcgccaaccagccgtccgccatggccggcgtcctggacatcaaggcgagcgagaagggcgcccggttcgtccagttctgcgactccttcagcatcccgctgatcaccctcgtcgacgtgcccgggttcctgccgggcgtcgaccaggagcacgacggcatcatccggcgcggcgcgaagctgctctacgcctactgcaacgcgaccgtgcctcgtatctcggtggtgctgcgcaaggcgtacggcggtgcctacatcgtgatggactcgcgttccatcggagccgacctgtcgttcgcctggcccaccaacgagatcgcggtgatgggcgccgagggcgcggcgaacgtggtgttccggcgggagatcgccgcggccgaggacccggacgcgatgcgcaagcagaagatcgacgagtacaagaacgagctggtgcacccctacttcgcggccgagcgcggtctggtcgacgacgtcatcgacccgcgcgagacccgctcggtgctgtgccgctcggtcacgatgctcatcgccaaggacgccgagctgccccgccgcaagcacggcaacccgccccagtagacccgacgaccacagagaggtgcacgacatgagcgagatgacccagttcaccgagcccgccgagcccgccgccgagtccgccgggatgaccctggagcactgccgcgagctgttgcgggtcgagcggggcaaccccgatccggaggaactcgcggcgctggcggcgctgttcttcgcccacttctccgcgatcgaggcgcggcgggaggccgcccgtgtcctgatcccgcggcagcggcgctccgcgagctggcgccgcaccgagcgggcacccggcttcgacggcccgcgcacctggcgcgcgggcggtcccgcactcgtttgacgatgcgtcaattgccgtaggaatacggcgatttacccaccacgacagtaagcgcgcaccccgtgcgggccgccccggcagggcggcaccgcccggggtgcgcccctgttcgggccctgcgggctttacttgacacgcgctcggccggacgaccattctgtgcggtgaatgggggtcttcgtcatgcaggtgttgccgtactgcaagctactgcccgatcacacggagcgccggggcgctccggcggcgcccgactgaggcgcaggcaggcgaaattcagccaacggccatcatggcgccacttggacacccgttatttaacacgccgtcagcagacccgccggaattctgtatacgggcaggccgatgtggcgaacgtcatgtcatgactctgccaacatcagagctgcgccaagatggccggtttatgcgagtcgcggcgagttacgcttggttgaatcgtcagttgtgccggtcttccgcttccgcatggcggggactggttgacgagggctcctgatcgtgatcacgtatggaaacgagcaggcagcctgtccgatcccgctcaataaccgacacagtttatttgttatttgctcgaacctctttgaattccgtagcagcggtctgtaggttcgtcggtgagatctggatcggatctcatattggactcactcagatgggggagggacatgactcgcgggggaggcatgactcagagttcgtcagaggcagtgctggagcctcatatacctgtccagcgtggtcccggtggaccacacctgatcgaagagggcgtgtcggaagcggtgcgcagggtggccggcctctcgcggggccggcggatcctcgtggtcgacagcgacgtcgacggcgcggagtccctggtgtgccggctgcgcaggcacggccacgaggccatcggcgtgaagagcggtagcaccgcgctgcaggcgtacgaggacgtggaccttgtcctcctcgacctcgaactcccggacctggacgggctggaggtgtgccgggccatccgctccgtgagcggcatccctgtgatcatcgtcaccgcccggggctccgagctcgactgtgtgctcggcctacaggccggtgcagacgactatgtggtcaagccctatggcttccgggaattaatggcacggatcgaagccgtcatgcgtcgcgccaggttccaaccgcctgttgccagagagatcttgcacgggcggttgcgcattgacgtgagctcccgcgaggtgagcctggacggccgcgaggtggggctgacccgcaaggaattcgatctgctctgcctgctcgcgtcccatccggacacggtcattccgcgaaagcgcctgctccagcaggtctggggggactcctggtcccgccgtactgtcgacacccatgtcagcagccttcgcggaaaactcggcgacagcggctggatcattactgtgcgcggggtcggtttcaagctgggcaacgggtgaatttccgtctttccgcccggtgtgcgcaacggaaaaacaagttgaacaaccgtcttatatccgaagaattctttggtgccgcagccggtaaatgattgccgagtgtgagacgggttttcgggcggggttccgggggtattgtttctcccctggggccccgccttctttatcagtaaccgcagtaaatatcaattccgtgcaacatgccgtctcgcacaccttggcaggcccctgaacaacacactgaacacagcgaaaaggccccccgcggccggccgggaagccgggccgcgggggccttttcgtgaactgccgtcgtcgccctacgggttgagcacccaggcggagttgtgctgggtgaagccgatgtgcgggtagtactccaccgccgccggcgccgacaggagaatgatcttcgcctgcggggcctccttctgcgtggcgtcgatgagcgcgcggccgatgcccgagcgctggtagtcgccgctcaccgcgatgtccgagaggtacgtcgcgtaggagaagtcggagatgctgcgggcgatgccgatgagcctgccctccgcgtcccgcgccaccaccacgaggttggcgttgcggaccatggcggcgaaccgctccacgtcctcgatgggacggcgctcgccgagcccggagctgcggtagacgtcgaggaccgcctccaggtccaggtcggcgccctccacccgctcagtcgtccaggtcacgaagctgctccaatcgcttgagaatcacaccgtggttgacgaggaaacgctccgggcggagctggccgatgtccatgccctcctgccgcagcgtgtccccgaagtgctcgccggtcgcgatggtgcggttggcggcggactggacggcccggtaggccttctcccgctccaccccgtcggccagcagatcggcgagtacggcagagctgaacacaagaccgtcggtctggtcgatccccgcccgcatccgctccgggaacaccttgaggttacggaccaggtcggccgccatcgtggcctggaagtgccccaccgacagcgcgtccggcaggatcacccgctccaccgactggtgggccagatcccgctcgtgccacagcgccacgttctccagggccgtggtcgcgtaaccgcgcagcagccgggccagacccgtcagacgctcgctggtggtcgggttgcgcttgtggggcatggcgctggagccctggtacgccgaggtgcgctgctcctcgacctcgcggacctcggtgcgctgcagcagccgcagctccagggcgatctgctcgacgctcgcgccgagcacggcgacggcctggatcagctgggcgtgccggtcgcgggcgacgacctggctcggggccggctccacccccaggtccagctcctcgcagacgtacgcctcgacggaggggtcgatcagcgcgtacgtgccgaccgagccggagatcgtgcccaccgccacggccttgcgcgccgcgcgcagccgggtgatcgagcggtccaccgcgaacgcgaactgcgccagcttgtggccgaaggacgtcggctcggcgtggacgccgtgggtgcggccgacgatgaccgtctcccagtgctccagggcccgttcgaccaggaccttgcgcagctcgaccgcggccgcgatcaccaggtcggtggcgcgggccaggttgtagcccagcgaggtgtcgacgaggtcgtagctggtcatgccgaggtggacccagcgggccgactcgtccgggatgtcctcgcagtacgcggcgaggaacgagagcacttcgtggtcgcgctcgcgctcgatctcctgcacccgctcgggcgtcgggaccttggcccgccgcatgtcctcgaccgcgtcctcgggcacccgtcccaggcgcgcctgcgcctcggaagccaggatctccacccggacccaggtcgcgtaccgcgcctggtccgagaagatgtccgccatcgcgggcagggtatagcggggaatcatgtgggccagcacccccagggtgagcttgtcgacaagtggatcggacggaccggcagtggtgcacgggccgtcgtgaggggacgggccgtcatcagaaggcctcgaagtattcgcgctgctcccactcggtgacctggccgtcggcgggccgctcggccgcgcgccaggcctcgtagcgggacagctcgctctccttcagcttggccagggtggcggccagcggcttgccgagcagttcggcggcctgaccggcccggaaggcctccagcgcctcgccgaggtcctgcggcagcgtctcgtatgactcctcgccgctctcccgcgtgctcgcgccgacctcggtggccatgccgtcgaacccggcggagagctgggcggcgatgttgaggtacgggttggcggtgggctcgcccacccggttctcgatgtgcgtgccggcgccgccgccgaccacccggatcatcgcgctgcggtcctcgtagctccagccgagacgggtcggcgagagcgagaagtccgcgcccatgcggcggtagccgttgacggtggggaccgagagcagacacaggtcgcgggcgcgggagagcagcccgtcgatgtacgccttgccctggtccgagatgccgccgccgtcggccgcgaagaggttgcgcccgttcgtgctgtccatcaccgactggtgcagatgccagccgcacgggtcgaagctgtcgacgcggggcagcgccatgaaggaggcgtggtagccctggcgcgcggcggtctgcttgaccacggtgcggaacaggagcatcgcgtcggcggtgtccagcgcgtgcatcgggttgaaggtcgtctcgatctggcccgggcccgactcgtgctcgatcgagcgcagcggcaggccgagttcgagcagcttcatcgccagcgggctggtgaagtgggccaccgagtcgtagttggcgtccaggttgaactggtagcccgagttcatcgcctcgaccttgggggccgcgccctggaggccgaagccgttgcccgcgttcccgggcggacccgcgagcttgcgggtcaggtaccactcgacctccaggccgagcaccggggtgaggtcgcgggccgcgtaccgggcgacgacctggcgcagcacgttccgcgcggagagcgggtgcggggagccgtcgcgcagatactcgtcaccgagcacccacgcggtgcgcggtccctcgtgggggagcacctggaacgtcagcgggtccgggaccagcacgaagctgcccgcgcccgcgatctcgtcgacgccgacgcccgggtcggcgaggaagtccagggcggccgcgtggccggtgtcgaagatgaacgggcccgagctgaagtccatgccgttgcgcaggaccgagcggaacgcgtccacggtcagcgtcttggagcgggccagcccgtgcgggtcggcgaaggcgaggcggaccaggtcgatctcgtcgagcgaggcctcgatctgctcggccgcagcggcctgttcgtcggtccacaggccgaactcggtgacgaacgcgggacgtcctgtgccgcccgcctcggagaaggggctggaccacgaccgggagtacatgggttacaggatcctttcggttactcggtccggtgcggccggcagcggcccgaggtccgcctccagacggcgtgcggcggacaggaccagatcgtccgcgccgaaggggcccacgagttggagccccaccgggagaccggcgcgggtgagtccggccgggagcgaaacggccggctggccagtcatattgaagggatacgcggcgggtgtccacgccagccagagcaggtcctccggacggctggcccagtcagggccgatcgcgtggggatcgatcggctcgatgggcacggtggccatcgcgagcaggtcgtaccggtcgaagatctggtgcagtgtggtgcgcagcgccagacgcacctcctcggcccgcatcacggtggccgcgctgagcgtgcggccgtgccgcacgatcgcgaggcggcccgggtcgcaccactcctcgtcggcgggcgaggtacccgccgcgtcgctcgcggcgaggatgtcgacgagcgccggatacgggtcgcggaacggcacctcgatccgctcgacgcggtgcccctgcgcggcgagcgcgtccagaccctgctcgctgacccggcggacctccggcgaggtgcccgggaactcgatccagccgatgcgcaaccgccgccgctggcggggcagttcatgcacgccgagcatcgagtccgggtcctgcggatgcccgccggtgatcaccgaggcgagctcgatgacgtccggcacggtgcgcgcgatcggcccctggtgggagagccggtcggcgcaggcgggcacatacggcaccttggcgaacgacggcttgtagccgaccacaccgcagaacgccgacgggatacggatcgagcccgcgccgtcggtgccgagcgcccccgagccgagccccgccgcgaccgcggccgcggcgccgccgctggagccgccggcggagagttccagatcccacgggttacgggtgggcggggccacccggctcaccgtggaggcgctccacccgtactccgacgtcgtggtcttgccgatgacgatggccccggcggcccgcagccgcgcgaccgagggcgcgtcggcccggggccgccggttctccaggagggacccacgggcagtgggcaggtccccggtctggatgaggtccttcaccgagaccgtgatcccgagcagcggctgccggtcgaagacggccgggccctcctcgcggatccgggcgtcggccagttcggcctcccgcacggcctcgtcgcccgcgaccgagacgaaggcgccgagctcgatgtcggtcttctggatcgcggtgaggaccgactgcacatggtcggcggcggacagctctccccgggcgaggaggtcgcgtatgtcgccaatggacgctgaggtcaacggagcagctcctcgtcagaacggggtgggccgccggtcacgagacctgctgcggctgcttggcgcgcgccgccgcggcctggaccgacttgaggtgccactcggagtcgaaggcggtgacctcgatctcctcgcccgcggccaccagcgcggcggcggcgctcatggcgccggcgatgaaccccgaggtgcggtgcgcggcgagcaggtcggactcgtcgaacggggtgccgtcgcagcgcagcagacgcaggtccatgaacccgttgaacctctggcccagcacctcgaccgactgcgggctgcggatgctgaaccgcaccttcgacgccacatggccgtggtggtgctggtcggagtagaaggcgacgtcggggatggtcggcgggatgaagtggtcgcgctccaccacggcgggcaccttgggcaggttcccggcgatgaagtgccaggagttgtactgcatgcgggacgacatcgcccaggcgttgtcggccagttgcagcaccgagtcctcgtagtggcgcttggccgacggggcgggcaccacgcagcagaagaagtcggtgatgtcccacttggtgatctcggcgtagctctgctcccgcagggcgcgcatcagttcggggatcgaacgcatgccccggctcatggcgaagtcggcctcgaagacctccgtcgccccggccaccgtctcgtacaccagggcctcaagtccggtaccgaactcgccgtacggccgggcgagccaggagggggacgcggccaggctcgcgcggagctcggcgaaggtggcgccggtgaccgggagacgctcgctcagcagggcggcgagctccgcctcgcgctgctgcgacttctccccgtacggaccggccaggcgctcgatcttgtgcatcagcgcgccgtggatctcgcggtagagctgggcgttggggatgacatggcccatgcggcgctcgcgcagcgccatggcgagggcgttcagcgcctcgacgcggtgcgtgggagcgggcgggacgagctcgccgccggggaccgcgttgtagttgccgcgggtgcgctccagcatgtacgcgatgtgatccagcgtcagctgggtgccgttggcctcctcgatgcgcccggcgcccgcggagcgcaccagcagcgtgaggcagacgaccatcaccgcgtcgtggtccgaccactgctccatggagacgcccatcagacggctgaacgtctcgttggggtggcccgcccacagcgtcttgccgatgacgttgttggtctcgcggaagttggtgtagagcttcccctcctggtgcagcacgaagggggcggtccgcagcgcggactcccgcagcatctccaggagttcgtcacgggcctgggtgccgagcgccgccagccactggcggcagtcgacgacctggtcggcgaagcgggcggccgcgcggtcgacctcgctctcgtagtcgagcaggtcctgcgccgaccacggcacgctcatgacgccctcgaccagcttctcctcgtcctccaacggcccctgcgagggcacgcggacgacgatgcggccggtcgtggtcaggcccagctcgccgaggaacgcggggcgctccttgacgtcgcgcacgggcgtcggcaggttctcctcgcgccacgagatgccgcagagcaccttgagcgcggcctcgtcggcggtgcgcagggcgcgcagctggacctcggcggggacgtggccgtccagggtgagcagggcgttcaccgcgccgcgcaggtccggggccgcggtcgcgcgctcccaggcgcgcttgagcagagtggggaaaccggcctcctcgtctttgaggcggtccggcttcggcgtggccccggccaccgcgccctggcggctcggccggcggctctgccgcttcttggcgttgatggcccggcgggaggtgccgttgcgctcggcggaacttgtcatgacacaccctcagcggtaagcccgatgtgcttccacatctcgtcggtcgtgggcgtccagtcctcgtcgacgtagatgcagtcgaccgggcactccaggacgcacttggggcagccggagcacagttcggggatgatgacgacgtcgagtccgttgtcgaagatcgcgttgaactcgggcgggcaggcacgcaggcaggtgtcgcaggtgatgcactccgcgcgctcgatacggcgtggcggcttcttccacgtttcgctgcgggtccgctcggcgatcagcttgtcgcgcccggacaccgagccttccactgtctcgtccagcgagtcgtccgaagtgttcgtgggcattcggcccccctcttgtcctgtttcgctcgtgccacggtaagagagcattaataactgcgccgcagtgcggcgacgacggccagaattacacgcggccccgctccatgggaacggcggacgagcggtgtgcagacgcggatttcatggctgcggtggattcctgctagagggaatcttgaacccgcgggcttctgataatccaccgttccgcaaggggacgcaatatgctcaagtcaccgcgccgacgtccgtcacaagacggcgcgggggatccgtgtcggtttcgttgatacccgctctgacctgctgcgattgggtgatccgggcttcggcttcgcccccgcgggccggaccggggcgggccccgtcgggcgacggtatacgcgaacggcggccgtgcaaccgcggaactcacgcagtcgaacaccgcgaccgggtgcactccggtttgtcaactccgtgcacggaaacccactggagtcaccctaccgttttactggtgttgtgtccgtcatcggatacgcgtgtatatcgcacgcggtgaatgctccctaaacggagatatccgcgttatggccgggggaaagggaatccattgtgcgggaacagcagcagtgcgttggaacctggcgcgacgcatgacggnttcgatccgtnnnnnnnnnnnnnnnnnnnnnnnnnnnnnnnnnnnnnnnnnnnnnnnnnnnnnnnnnnnnnnnnnnnnnnnnnnnnnncggcggcgccactggtgttgggggcgtgtcacctcaagttccgtcgccgtaagcccagttcatgagggggactgggggagtcatggcgcggaagggggcacacgcgggccgctcggcggccggccctcagtcgttcaggatcgtcagatcgagccggtccagccgctcgggcgcggcccgcatgtcgatcccggtgatcttctcgtccacgacggtgaacccgaagacgaccctcggccgcccgcgcggcgcccacaccgccccgacgacgccgtcgaccagcgccagcttggcgtacttggcgcgccccgagaacgcgtccgccaccgagcccgcgccccgcacctccgtcggcgcccccaccgccaccgcctccgggtcggcgcggagcaccacgtccggggcgagcagcgcggtcagggtggcgaagtcgccgccgcgcgaggcggtcaggaacgcgtcgacgatccggcgcagccgcgtcgtaccgtcgtccggcagcggatcgacccgccgcacccgcgcccgggcccggtcggccagctcgcacgcctcctcggcgccggtccccacgatcggcgccacctcgtcataggggaggccgaacagatcgtgcaggacgaacgccatccgctcggccgggtccagcgtctccaggacgacaaggagggcgaggcccaccgagtccgccagcacctcctcgtgcgcggggcccatcgaggcgagtttggcggccagttcggccgggaccgggtcggccagatccaagcttttcgatcagaaacttctcgacagacgtatatcaggcttcccgggtgtctcgctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcctgccg

SEQ ID NO: 2 - DNA, 1785 bp, Streptomyces murayamaensis ATCC 21414
gtgagtccggaacgtctcaattccgtggacttccgcgaccaggtgctgcccaagctcgcggccaccaccggtctgctggtggtggacgaggcgcactgcatctccgactgggggcacgacttccggccggactaccggcggttgcgggcgatgctgaccgagctgcccgccggggtgccggtgctggccaccaccgcgaccgcgaacgcccgggtcaccgccgacgtggccgaacagctgggtaccggggccggtgaggcgctggtgctgcgcggcccgctggagcgcgagagcctgcgcctgggcgtggtccggctgccggacgccccacaccgcctggcctggctcgccgagcacctcgacgagcttcagggctccgggatcatctacaccctcaccgtggccgcggccgaggaggccaccgccttcctgcgccagcgcggcttcaaggtgtcctcgtacacggggcgcacggagaacgccgaccgtctgcaggcggagaccgacctccaggagaaccaggtcaaggcgctggtggcgacctccgcgctcggcatgggcttcgacaagccggacctcggcttcgtgatccacctcggctcgccgtcctcgccgatcgcctactaccagcaggtcgggcgcgccgggcgcggggtcgagcacgccgacgtcctgctgctgccgggcaaggaggacgaagccatctggcgctacttcgccgacacggccttcccgcccgaggtccaggtccgccagaccctgtcggccctcgccgacgcgggacggccgctgtccgtaccggccctggaggcggcggtcgacctccggcgcagcaggctggagacgatgctgaaggtgctggacgtcgacggcgccgtgaagcgggtgaagggcggctggacggccacgggggcggactgggtgtacgacgccgagcgctacgcctgggtggcccggcagcgggcggccgagcagcaggccatgcgcgattacgtgagcacgacgcggtgccggatggagttcctgcgccggcagctggacgacgagggggcggccccgtgcggccgctgcgacaactgcgcgggagcgtgggccgattccgccgtgtcggcggagacggtgacgggggcggcgaaggaactggaccgcccgggggtggaggtcgagccgcgccggatgtggccgacggggatggccgcgctgggcgtcgacctcaaggggcgcatcccggccaaggagcagtgctccaccggacgcgccctggggcgcctgtcggacatcggctggggcaaccggctgcgcccgctgctggccgagaacgcgccggacggacccgtcccggacgacgtcctgcgggccgcggtcgcggtcctcgccgactgggcgcgctcgccgggcggctgggcgcccgacgtcccggacgccgtcgcccgtccggtgggagtcgtcgcgatgccgtccctggcccgcccccgactggtcgcctccctcgccgaggggatcgcgacggtcggccgcctgcccttcctgggcaccctgacgtacacgggcccggacgacgcgcacgcggcgcggcgcagcaactccgcgcaacgcctgcggacgctgtcgggtgccttcaccgtctccgaggacctggccaccgcgctggccgccgcccccggccccgtcctgctggtggacgactacaccgactccggctggaccctggcggtagccgcccgcctactgcgccgcgcgggcagcgaccaggtcctccccctggtcctggcggctacgggctga

SEQ ID NO: 3 - protein, 594 aa, Streptomyces murayamaensis ATCC 21414
VSPERLNSVDFRDQVLPKLAATTGLLVVDEAHCISDWGHDFRPDYRRLRAMLTELPAGVPVLATTATANARVTADVAEQLGTGAGEALVLRGPLERESLRLGVVRLPDAPHRLAWLAEHLDELQGSGIIYTLTVAAAEEATAFLRQRGFKVSSYTGRTENADRLQAETDLQENQVKALVATSALGMGFDKPDLGFVIHLGSPSSPIAYYQQVGRAGRGVEHADVLLLPGKEDEAIWRYFADTAFPPEVQVRQTLSALADAGRPLSVPALEAAVDLRRSRLETMLKVLDVDGAVKRVKGGWTATGADWVYDAERYAWVARQRAAEQQAMRDYVSTTRCRMEFLRRQLDDEGAAPCGRCDNCAGAWADSAVSAETVTGAAKELDRPGVEVEPRRMWPTGMAALGVDLKGRIPAKEQCSTGRALGRLSDIGWGNRLRPLLAENAPDGPVPDDVLRAAVAVLADWARSPGGWAPDVPDAVARPVGVVAMPSLARPRLVASLAEGIATVGRLPFLGTLTYTGPDDAHAARRSNSAQRLRTLSGAFTVSEDLATALAAAPGPVLLVDDYTDSGWTLAVAARLLRRAGSDQVLPLVLAATG

SEQ ID NO: 4 - DNA, 516 bp, Streptomyces murayamaensis ATCC 21414
atggtcggctcctgtgtggccgcgctcctggctcggttccccggtgtccgcgtgcagctcgtcgacgtcgaaccggcccgccaggcggtcgccgcgcggctcggcgtcgccttcgccccgcccgagcgggccactgacgactgcgatctcgtcttccacgccagcgccaccgaggcggggctgaggcgctccctcgaactgctcgctcccgagggctgtgtggtcgatctgagctggtacggcgaccggccggtgacgctgccgctgggggagttcttccactcccggcggctgtcgctgcgcggcagccaggtcggcgccatcgcgccggagcgccgcgcacgccacacgagggccgatcggctcggcatggcgctcgacctcctgcgggacccggtcttcgacgtactgatcaccggcgagtccgatttcgacgaccttccgcaggtcatggccgagatcgccaccggcgcccggcccgggctctgccaccgcatccgctacggccccggctga

SEQ ID NO: 5 - protein, 171 aa, Streptomyces murayamaensis ATCC 21414
MVGSCVAALLARFPGVRVQLVDVEPARQAVAARLGVAFAPPERATDDCDLVFHASATEAGLRRSLELLAPEGCVVDLSWYGDRPVTLPLGEFFHSRRLSLRGSQVGAIAPERRARHTRADRLGMALDLLRDPVFDVLITGESDFDDLPQVMAEIATGARPGLCHRIRYGPG

SEQ ID NO: 6 - DNA, 399 bp, Streptomyces murayamaensis ATCC 21414
atgttcagcgtcaccgtccgcgatcacctcatgatcgcccacagcttcagcggcgaggtcttcggccccgcccagcgcctgcacggagcgacatacctggtcgacgcgaccttccagcgaccggagctggacgacgacaacatcgtcatcgacatgggcctggcaggcagggaagtgcgcgggatcgtcgcggcgctctcgtaccgcaacctcgacgaggaccccgacttcgccggaaccaacaccaccacggaattcctggccaaggtcatcgccgaccgcctcgccgcccggatacacgacggcgccctcggccccggcgcccagggcctgaccggactcaccgtacggctccatgaatcgcacatcgcgtgggccgactaccaccgcacgctctga

SEQ ID NO: 7 - protein, 132 aa, Streptomyces murayamaensis ATCC 21414
MFSVTVRDHLMIAHSFSGEVFGPAQRLHGATYLVDATFQRPELDDDNIVIDMGLAGREVRGIVAALSYRNLDEDPDFAGTNTTTEFLAKVIADRLAARIHDGALGPGAQGLTGLTVRLHESHIAWADYHRTL

SEQ ID NO: 8 - DNA, 735 bp, Streptomyces murayamaensis ATCC 21414
atgaatcgcacatcgcgtgggccgactaccaccgcacgctctgaccgcccggtcatggccatacgcccgtacgtcgtgctgtccgcggccgtctccctggacggccggctcgacgacacctcgcgcgaccggctcgtactctccaaccggcgcgacctcgaccgtgtcgacgatgaacgcgccgccgcggacgccatcctggtcggcgccaccaccctgcgcagggacaacccgcgcctgctggtggcgagcgccgatcggcgggcccggcgcgtcgccctcggcatgccggagcatccgctgaaggtcacggtcaccgggtccgccgaggtgaacacgtcgtacgcgttctggcattgcggcggcgagaagctggtgttcacggtggacggagcgctcctgcgggcccgtcgcaccgtaggcgacctcgccgacgtcgtcagcaccggccccgctctcgactggcacctcctcctcgacgaactcgggcgacgtggggtggaacgccttctggtcgaaggcggcgggacggtgcacacccagctgctggcccaggacctggccgatgaactccacctcgtcgtcgccccgttgctggtgggcgaagccggtgcgccggtgttcctgggcccggcgtcgtacccgggtggtcccgcggcccgtatgacgctgctcgaagcacgccccgtcggtgatgtcgtgctgctgcgctacgccccgaaactccgatcctga

SEQ ID NO: 9 - protein, 244 aa, Streptomyces murayamaensis ATCC 21414
MNRTSRGPTTTARSDRPVMAIRPYVVLSAAVSLDGRLDDTSRDRLVLSNRRDLDRVDDERAAADAILVGATTLRRDNPRLLVASADRRARRVALGMPEHPLKVTVTGSAEVNTSYAFWHCGGEKLVFTVDGALLRARRTVGDLADVVSTGPALDWHLLLDELGRRGVERLLVEGGGTVHTQLLAQDLADELHLVVAPLLVGEAGAPVFLGPASYPGGPAARMTLLEARPVGDVVLLRYAPKLRS

SEQ ID NO: 10 - DNA, 366 bp, Streptomyces murayamaensis ATCC 21414
atgctcctcgggcgcgtggccggacggtgtgcggacgagacgggggcgagtgtggacgacgcggtggaactggcggagatgatcggccagttgcggagcgagctgagccgggccatggcggacggggccgcgggcggcgggctgcggttccaggcggagaagctggagctcgaactcaccgtgggcgtggagcgcagccgggagccgggtgcgaaggtgcggttctgggtgctcgacgtccacggctccgcccgctccgcgcggaccgccacgcagcggatcaagctcaccctgcaaccggtgctcggcgacgcacccgacagccccgcgctgatctcgggcgcggagctgcccgatgagagctga

SEQ ID NO: 11 - protein, 121 aa, Streptomyces murayamaensis ATCC 21414
MLLGRVAGRCADETGASVDDAVELAEMIGQLRSELSRAMADGAAGGGLRFQAEKLELELTVGVERSREPGAKVRFWVLDVHGSARSARTATQRIKLTLQPVLGDAPDSPALISGAELPDES

SEQ ID NO: 12 - DNA, 2949 bp, Streptomyces murayamaensis ATCC 21414
atgagagctgaaccgtcggccgacgggctcgacccgcaccggatcgccgagatcatcgtcgagagccccgagggcagaaggcgcggttccggctaccggatctccaccacgacggtcctcaccgccgcccatgtggtcgccgacgcgacccgcacgctcgtacggtgcgacgccgaccagcccgcggagtggtcggctccggccatggtgacctgggcggatgcgggcagcgacctcgccgtgctgagcgtcggggcacctgcggccgcccccgtggtcaccgcacccgcccgcttcgcgcgcatcgccgacgaccggcacggggtgatcggggtgcacgccgccgggtttccgctgtggaaacgccgacgccggtccgacggcgcgtacttccgcgaactgcaccaggcggacggcacggtggcggcgctctccaatctgcggaccggcacgctggagatgaccgtggcaccggccggaaccgaccccgacccggcggcctcgccgtgggcgggcatgtcgggcgcggcggtgtgggccggcagccgcatcatcggcgtggtcgccgagcaccaccgctacgagggccccggacggctcaccgcggtccgcctcgaccacgcgctgcgcgggctcggcgccgcgagacgggcggagctggcccggttgctcgcgctgcccgagaccgcggatctgcccctcgccgtccccgggcaaccgcacgacgaccgcgccccgggggtgcgggtggtcggcgtccccgtcgcgcacggcatcgagctgttcaagaaccgcacccgcgaaagcgacctgatcgcccgtcacttgagcgatccggccatccgtatggtgtccgtcgtcggacggcgcggcatcggcaagagcgcgctcgccgcgaaggtcatggatctgctggaccggggggaatggcccggcgcggccgccggtcccctcccgtccggcctcgtcaacctctccacccgaacctccggcatctccctggagcggctctacttcgactgcgtccggctgctcggccccgagcacgaggagcggctgcgcggggtctgggcgggcggcggcagcgcgcaggaccgcctcgccgcactcttcgacgccatgggcgggcggctgatcgtcatcctcatggacaacttggaggaactgctcggcgacgacggcggcatcgaggacgaggagctggccctcttcctggactggctgttccgggcccgcaccaccccacgtctgctcgtcaccagccaggtgccggtgcgtctcgcgcccgaactgcgccgcttcgccgccgaggtgccgctctccgaggggctgcgccccaccgaggccgcggccctgctgcgcgaactcgaccgggacggcagcctcggcatcgccgacctgtccgacggcgaactcctcgacgccgcggtccgggtgcacggcgtcccgcgcgcgctcgaactcctcgtcggcgcggtcgccgaggagacggtgctgctgcccagcctgaaagacgtactggaggacttcacccgccgccacgacgtcgtcgcggacctcgcccaggaccgctaccgtcgcctcgacgagccggcgcgtgccgtcctcggtgtgctcgccgccctgcgcacccccgtggagcagggcgcggtggcgcagatcgcgggcgggctcgatccggacctgcgggtggtccccgtcctcacggcgctcgtccgggtccgcctggtctccgtggaccgggccagccggacggtcgccctgcacccgttggacgcggacatcgcccgtgaccagatgccgtgggacggacctttcgggaggcaagcggtggaacggcagatcgcagcctggtacgcgcggcgggcgaagccgcgcggcgcctggcggacgctggaggacgtcgagccgcagcggcggcagttcgaccacctggtcggcgcgggcgaccacgacgcggccgcgcgggtgctggccgagatcagcgaatggctcgtctggcacggctcggtgctcgccgcggtcacgatgcacctggcggtcgacggacacctccgcgacgagcgggtacgcctcgcccacaccgtcgcgtacgggcacgcccggctcagtgcgggtcccatggagcaggcggtcgagctgttcaccgaggccgcggccctcgccgaacgcctcggcgaccggcccgcgctgcagaacgcgctgttcggcctgggcgacgcccaccggcagctgggcgatcagggcgccaccgtcgaaccgctcgcccgcgccgccgagttggcgcgtgaactgggcgacaccgagcgcgaggcgcacgcgctgctctccctcagcctcgcgcacagctacctcggcgacggcgagcgcgcgctggagggggccgaccggctggccgcgctcgccgaggcgggcggcgacccgctcgcgctcgcccgcgccggcaacgcgcgcaccatcgcgctgctcaccctgggccgctggcaggagaccgccgaggcgggcgccgagacggcccgcgcctatcgcgccgccggcagtcaggaagcggtcgcgtacgcgctgaacgcgcagggcctggccctgctggccctggacgacccggcgcgggcggccgcggtgctcgaagaggcccgccacgaggcctcactgatggagagcccccgggccgagggcgtctgcctgctcaacctgtcctgggcgtactggtgcgacggccgccgggacgagtgcgcggccaccgccgaacgcgcctcgaccgccctccagatcgccggagcgacccaggcggcggcggcccgttcgctggccgaggccgcccgcgtcctgcccggcgaccctggggccgccgccgacgccctgatccgcgcggcggccgcgctggacggcaacgccgaggtcatcgcccccgcccggctcaccgccgaggcacgccgactgctggactga

SEQ ID NO: 13 - protein, 982 aa, Streptomyces murayamaensis ATCC 21414
MRAEPSADGLDPHRIAEIIVESPEGRRRGSGYRISTTTVLTAAHVVADATRTLVRCDADQPAEWSAPAMVTWADAGSDLAVLSVGAPAAAPVVTAPARFARIADDRHGVIGVHAAGFPLWKRRRRSDGAYFRELHQADGTVAALSNLRTGTLEMTVAPAGTDPDPAASPWAGMSGAAVWAGSRIIGVVAEHHRYEGPGRLTAVRLDHALRGLGAARRAELARLLALPETADLPLAVPGQPHDDRAPGVRVVGVPVAHGIELFKNRTRESDLIARHLSDPAIRMVSVVGRRGIGKSALAAKVMDLLDRGEWPGAAAGPLPSGLVNLSTRTSGISLERLYFDCVRLLGPEHEERLRGVWAGGGSAQDRLAALFDAMGGRLIVILMDNLEELLGDDGGIEDEELALFLDWLFRARTTPRLLVTSQVPVRLAPELRRFAAEVPLSEGLRPTEAAALLRELDRDGSLGIADLSDGELLDAAVRVHGVPRALELLVGAVAEETVLLPSLKDVLEDFTRRHDVVADLAQDRYRRLDEPARAVLGVLAALRTPVEQGAVAQIAGGLDPDLRVVPVLTALVRVRLVSVDRASRTVALHPLDADIARDQMPWDGPFGRQAVERQIAAWYARRAKPRGAWRTLEDVEPQRRQFDHLVGAGDHDAAARVLAEISEWLVWHGSVLAAVTMHLAVDGHLRDERVRLAHTVAYGHARLSAGPMEQAVELFTEAAALAERLGDRPALQNALFGLGDAHRQLGDQGATVEPLARAAELARELGDTEREAHALLSLSLAHSYLGDGERALEGADRLAALAEAGGDPLALARAGNARTIALLTLGRWQETAEAGAETARAYRAAGSQEAVAYALNAQGLALLALDDPARAAAVLEEARHEASLMESPRAEGVCLLNLSWAYWCDGRRDECAATAERASTALQIAGATQAAAARSLAEAARVLPGDPGAAADALIRAAAALDGNAEVIAPARLTAEARRLLD

SEQ ID NO: 14 - DNA, 594 bp, Streptomyces murayamaensis ATCC 21414
atgcctacgtcaacgccagtcaccgagaacggtccccgccggctccgggccgacgcggagcgcaaccgggcccgggtgctgaacgcggccagggagctcttcgcggaacgcggcgccaacgtgtccatggacgaggtggcccgccgcgccgaggtcggcgtggggacgctctaccgccacttccccaccaaggaagccatggtcgtcgcggcctcccagcagcgcttcggcgagatcctcacgtactaccgcacggtctgccgggagtcggccgagccgctggaagcactccagatgctgctcacccgggtcggcgaggtggaggcccgggaccgcggcttcgccagcgtcgtcggcgggacgctcggctccgaggggccgcccagcaccatgcgggccgacctggaggacgagctggtggacctggtcggcaagggccaggcggccggctccatccgcaccgatatcgcgggcgccgacatcctcgcgctgacctgcggtctcacctcgatcgtgcaccggcgcagcggcgactggcggcgctacatcgacatcgtgctcgacggattgcgctccgaccgcgccacgtcctag

SEQ ID NO: 15 - protein, 197 aa, Streptomyces murayamaensis ATCC 21414
MPTSTPVTENGPRRLRADAERNRARVLNAARELFAERGANVSMDEVARRAEVGVGTLYRHFPTKEAMVVAASQQRFGEILTYYRTVCRESAEPLEALQMLLTRVGEVEARDRGFASVVGGTLGSEGPPSTMRADLEDELVDLVGKGQAAGSIRTDIAGADILALTCGLTSIVHRRSGDWRRYIDIVLDGLRSDRATS

SEQ ID NO: 16 - DNA, 420 bp, Streptomyces murayamaensis ATCC 21414
atgtcgttgcccctggggcgtacgagtgcgccggaggtgtcggcgtcggtggaggcattcctgacccagacgccgcacatcggcgtgctgaccaccatccggcccgacgggtccccgcatgtggcgccggtgcggttcacctgggacgcggaggcgggtctcgcccgggtgatgacggtgtcctcgtcccgcaaggcgcgcaatctgatcgccgcgcccggcagccgggtcgccatatgccaggtggccgggttcgcctgggtcaccctcgaaggctccgccgtggtggccgacgacccggtgcgggtcaccgagggagcgcgccgctacacccgccgctaccgctccgggccgcccaacccgcccggccgggtggtcgtggagatctcggtcgaccgggtcatgagcctcaacgtctga

SEQ ID NO: 1 - protein, 139 aa, Streptomyces murayamaensis ATCC 21414
MSLPLGRTSAPEVSASVEAFLTQTPHIGVLTTIRPDGSPHVAPVRFTWDAEAGLARVMTVSSSRKARNLIAAPGSRVAICQVAGFAWVTLEGSAVVADDPVRVTEGARRYTRRYRSGPPNPPGRVVVEISVDRVMSLNV

SEQ ID NO: 18 - DNA, 447 bp, Streptomyces murayamaensis ATCC 21414
atgacctcaagccaatccaccgccgatgtcgcgggacttctggaccgttacctgatcaatctggacgacgacaagctcgacgacgcctgggcccgggggctgttcaccgaggacgcggtcgtcgagttcccgatgagccggcacgaggggatcgccggactcgccgagtaccacagcacggcgctcgcggccttcgcgcgcacgcagcacatcggttcgcccgccgtcgtggaaatcgacggcgaccgggcctctttgcggtgcaacatcgtctccacccatgtgcaccacccgtccgaccatccggaggacgccgaccgcgatccgatattcgccaacggaagcctggtgaccgccgaggcccgccgcaccccggagggctggcggctcagcctgctgtccctgcgcatgatctgggtgaccggcaccgcgccccgcaagggctga

SEQ ID NO: 19 - protein, 148 aa, Streptomyces murayamaensis ATCC 21414
MTSSQSTADVAGLLDRYLINLDDDKLDDAWARGLFTEDAVVEFPMSRHEGIAGLAEYHSTALAAFARTQHIGSPAVVEIDGDRASLRCNIVSTHVHHPSDHPEDADRDPIFANGSLVTAEARRTPEGWRLSLLSLRMIWVTGTAPRKG

SEQ ID NO: 20 - DNA, 1470 bp, Streptomyces murayamaensis ATCC 21414
atggataactttgacgcggacgtaattatcgcgggagccggccccactggactcatgctcgcaggcgaactgcgcctgaacggcgtgtccgtgattgtcgtcgatcgccttgccgagccgatacagcagtcgcgtgcgctgggattttcggcccgtaccatcgaggaattcggccagcgcggactgctgtcccgtttcggtgaagtcgacgtcattccggtcggtcatttcggcggagtgtccatcgattaccgtctggtcgagggcggttcgtacggagcgcgcggcattccccagtcgcgcaccgagggcgtgctggcgggctgggccggacagctcggcgccgaggtgcggcgcggggtcgaggtcacgggcctggacaacggcgccgacggcgtgagcgtggaggtcagcaccgccgaggggcccgccacgctgcgcggccgctacctggtgggcgcggacggcgcccgcagtgcggtgcgcaagctcgcgggcatcgacttcccgggcaccgacccggccatcgagctccggttcgccgacatcagcggggtgccgttgcgcccgcgcttcagcggcgagcgggtgcccggcggcatggtcatggtgctgccgctcggcccggagcgctgccggatcgtctacttcgaccgcagcgagccgttgcgcaagagcccggacccgatcaccttcgacgaggtggccgaggcgttcaagcggctgtccggcgaggacatcagcggcgccaccgtgcactgggtctccaccaccaccgatgtgagccggcaggccgccgagtaccgcaggggccgggtcttcctggccggcgacgccgcccacatccatctgcccatcggcgcccagggaatgagcgccggcgtgcaggacgcggtcaacctcggctggaagctcgccctggagatcaagggccaggctcccgagggcctgctcgacacgtaccactccgagcggcacccggtcggcgcgcgggtcctcaccaacacgctcgcccagcggatcctctacctcggcggcgacgagatcaccccgctgctcgatgtgttcaccgagctcaccgggttcgaggacgtccagaagacgctgatcggcatggtcaccggcctggacatccgccatgacgtgggcgagggcgaccatccgctcctcggccgccgtctcaaggacgaggagctggtggtcgacggcaagaagaccaccaacttcgaactcctcgcggccggcaaggccgttctgttcaacctcaccgatgacccccacctgcgggagctcgccgcgggctgggccgaccgtgtcaccacggtcaccgccgagcagcacgactgcgacaacggtctggacgccttcctggtccgtcccgacggctatgtcgcctgggtcgccccttccgcgtcgcgcacggaggggctcgccgaagcactcaaccgatggttcggccggcccaactga

SEQ ID NO: 21 - protein, 489 aa, Streptomyces murayamaensis ATCC 21414
MDNFDADVIIAGAGPTGLMLAGELRLNGVSVIVVDRLAEPIQQSRALGFSARTIEEFGQRGLLSRFGEVDVIPVGHFGGVSIDYRLVEGGSYGARGIPQSRTEGVLAGWAGQLGAEVRRGVEVTGLDNGADGVSVEVSTAEGPATLRGRYLVGADGARSAVRKLAGIDFPGTDPAIELRFADISGVPLRPRFSGERVPGGMVMVLPLGPERCRIVYFDRSEPLRKSPDPITFDEVAEAFKRLSGEDISGATVHWVSTTTDVSRQAAEYRRGRVFLAGDAAHIHLPIGAQGMSAGVQDAVNLGWKLALEIKGQAPEGLLDTYHSERHPVGARVLTNTLAQRILYLGGDEITPLLDVFTELTGFEDVQKTLIGMVTGLDIRHDVGEGDHPLLGRRLKDEELVVDGKKTTNFELLAAGKAVLFNLTDDPHLRELAAGWADRVTTVTAEQHDCDNGLDAFLVRPDGYVAWVAPSASRTEGLAEALNRWFGRPN

SEQ ID NO: 22 - DNA, 690 bp, Streptomyces murayamaensis ATCC 21414
atgcccaagatttcctctgatgacaagcacctgaccgtcctcaacctgttctccacggacgccccggagaagcaggagggtctgctcggcgcgatgcgcgagatcgtcgacgcggccgcctacccgggctggatgtcgtccaccgtgcacgccggcgtggacaagccgggcacggccaacttcatccagtggcgcagccgcgcggaccttgaggaccgctacgacggcgaggagttcaagcaccgcacgctccccctcttcggcgagctgaccacctcgatccggctgctccagaacgaggtcgcgtactcgcagaccaagtcgggcgacagcgtcgagatctccccggcccgcaccgacttcaccgtcatcgcggtcttcggtgtcgaggagaagaaccaggacgacctggtcgacgcgctcggcccgtcgatgaagttcctcagcgacgttcccggctacgtctcgcacaccgtcctgaagggcatcgcggcccgtggccttgagggctccttcgtggtctcctactcgcagtgggagagccaggaggccttcgtcgcctaccaggccgtcgcgcaggccgacaagcccgccgcccgccaggacgcggagaagcgcaccggctcgctcctgacgtcggtggactccaacacctaccgcgtggtccacacccgcgcggccggcgagtaa

SEQ ID NO: 23 - protein, 229 aa, Streptomyces murayamaensis ATCC 21414
MPKISSDDKHLTVLNLFSTDAPEKQEGLLGAMREIVDAAAYPGWMSSTVHAGVDKPGTANFIQWRSRADLEDRYDGEEFKHRTLPLFGELTTSIRLLQNEVAYSQTKSGDSVEISPARTDFTVIAVFGVEEKNQDDLVDALGPSMKFLSDVPGYVSHTVLKGIAARGLEGSFVVSYSQWESQEAFVAYQAVAQADKPAARQDAEKRTGSLLTSVDSNTYRVVHTRAAGE

SEQ ID NO: 24 - DNA, 330 bp, Streptomyces murayamaensis ATCC 21414
atgcacagcacgctcatcgtcgcccgcatggaacccggttcgagcaccgacgtcgccaagctcttcgccgagttcgacgcgacggagatgccgcaccggatgggaacgctgcgccgccagctgttctcctaccggggcctctacttccacctgcaggacttcgacgcggacaacggcggtgagctgatcgaggccgcgaagaacgacccgcggttcatcgggatcagcaacgacctgaagccgttcatccaggcgtacgacccggccacctggcgctcgcccgccgacgccatggccacgcgcttctacaactgggaggggcgcgcgtga

SEQ ID NO: 25 - protein, 109 aa, Streptomyces murayamaensis ATCC 21414
MHSTLIVARMEPGSSTDVAKLFAEFDATEMPHRMGTLRRQLFSYRGLYFHLQDFDADNGGELIEAAKNDPRFIGISNDLKPFIQAYDPATWRSPADAMATRFYNWEGRA

SEQ ID NO: 26 - DNA, 1275 bp, Streptomyces murayamaensis ATCC 21414
gtgacaacgcctcgcagggtggtcatcaccgggatggaggtcctcgcccccggtggcatcggcaccaagaacttctggagcctcctcagcgagggccgcacggcgacccgggggatcacgttcttcgaccccacgccgttccgctcgcgggtggccgccgagatcgacttcgacccgtacgagcacggtctgagcccgcaggaggtccgccgcatggaccgggccggccagttcgcggtcgtcgcctcgcgcggcgcggtcgccgacagcggtctggagctcgccggcctcgacccgtaccgggtcggtgtcacggtcggcagcgcggtcggcgccaccatgggcctggacgaggagtaccgggtcgtcagcgacggcggccggctcgacctcgtggaccaccagtacgcggccccgcacctctacaaccacctggtgccgagctcgttcgcggccgaggtggcctgggcggtcggcgccgagggccccagcaccgtggtctccacgggctgcacctccggcatcgactcggtcggctacgccgtcgagctgatccgcgagggctccgccgacgtcatgatcgccggatcctcggacgcgccgatctcgccgatcaccatggcgtgcttcgacgcgatcaaggccacgacgaaccgttacgacgagcccgagacggcctcgcggccgttcgacaactcgcgcaacggcttcgtcctgggcgagggcaccgcgttcttcgtcctggaggagctggagagcgccgtcaagcgaggcgcccacatctacgcggagatcgccggctacgccacgcgctccaacgcgtaccacatgaccggactgcgccccgacggcgcggagatggccgaggcgatccgcgtggcgctggacgaggcgcggatgaacggcgacgagatcgactacatcaacgcccacggctccggcaccaagcagaacgaccgccacgagacggcggcggtcaagcggatcctcggtgaccacgcctaccggacgccgatgagctccatcaagtcgatggtggggcactcgctcggcgcgatcggctccatcgagatcgccgcgtccgcgctcgccatggagtacaacgtcgtaccgcccacggccaacctgcacacgcccgaccccgagtgcgacctggactacgtcccgttgaccgcccgcgaccgcaagaccgacgcggtcctctcggtcggcagcggcttcggtggattccagagtgccgtggtgctcgcccgtcccgagaggaagctcgcatga

SEQ ID NO: 27 - protein, 424 aa, Streptomyces murayamaensis ATCC 21414
VTTPRRVVITGMEVLAPGGIGTKNFWSLLSEGRTATRGITFFDPTPFRSRVAAEIDFDPYEHGLSPQEVRRMDRAGQFAVVASRGAVADSGLELAGLDPYRVGVTVGSAVGATMGLDEEYRVVSDGGRLDLVDHQYAAPHLYNHLVPSSFAAEVAWAVGAEGPSTVVSTGCTSGIDSVGYAVELIREGSADVMIAGSSDAPISPITMACFDAIKATTNRYDEPETASRPFDNSRNGFVLGEGTAFFVLEELESAVKRGAHIYAEIAGYATRSNAYHMTGLRPDGAEMAEAIRVALDEARMNGDEIDYINAHGSGTKQNDRHETAAVKRILGDHAYRTPMSSIKSMVGHSLGAIGSIEIAASALAMEYNVVPPTANLHTPDPECDLDYVPLTARDRKTDAVLSVGSGFGGFQSAVVLARPERKLA

SEQ ID NO: 28 - DNA, 1212 bp, Streptomyces murayamaensis ATCC 21414
atgacgtcgtccgtggtggtcaccggcctgggggtggcgtcccccaacggactcggcatccaggactactgggcggcgaccgtcggtggcaagagcggcatcggccgtatcacccgcttcgacccgtcgtcctacccggccaagctggccggcgaggtcccgggcttcgtcgcggaggacctgctgcccagccgcctgctcccgcagaccgaccgggtcacccggctggcgctcgtcgccgccgactgggcgctcgccgacgcgggcatcaccccctccgaactcggcgagttcgacatgggcgtggtgaccgcgagcgcggccggcggcttcgagttcggccagggcgagctccaggcgctgtggtccaagggcagccagtacgtctcggcgtaccagtccttcgcctggttctacgcggtcaacagcggccagatctccatccgcaacgggatgaagggccccagcggcgtggtcgtcagcgaccaggccggcgggctcgacgcggtggcgcaggcccggcggcagatccgcaagggcaccagcgtgatcgtgtccggcgccatcgacgcctcggtctgcccgtggggctgggtggcgcagctggccagcgaccggctctccaccagcgacgagccgacccgcgcctatctgccgttcgaccgcgacgcctcgggctatgtggcgggcgagggcggcgcgatcctgatcatggaggacgccgagtcggcccgcgcccgtggcgcccgtgtctacggcgagatctccggctacggctcgaccatcgacccgaaggccggctccggccgcccgccggggctgcgcaaggccatcgaactcgccctggcggacgcgggggtcgccccgggtgaggtggacgtggtcttcgccgacgcggccgccgaccccgagctcgaccggcaggaggccgaggccatcaacgccgtgttcggcacccgcggcgtgccggtcaccgctcccaagacgatgaccggacggctctactcgggcgccgccccgctggacctggccgccgccttcctcgccatgaaggacggtctgatcccgccgaccgtgcacatcgatccggccgccgagtacgacctggatctggtcaccggcgagccgcgcaccgccgaggtgcgcaccgcgctggtcgtggcccgcggctacggcgggttcaactccgcggtggtcgtgcgcgccgcgtag

SEQ ID NO: 29 - protein, 403 aa, Streptomyces murayamaensis ATCC 21414
MTSSVVVTGLGVASPNGLGIQDYWAATVGGKSGIGRITRFDPSSYPAKLAGEVPGFVAEDLLPSRLLPQTDRVTRLALVAADWALADAGITPSELGEFDMGVVTASAAGGFEFGQGELQALWSKGSQYVSAYQSFAWFYAVNSGQISIRNGMKGPSGVVVSDQAGGLDAVAQARRQIRKGTSVIVSGAIDASVCPWGWVAQLASDRLSTSDEPTRAYLPFDRDASGYVAGEGGAILIMEDAESARARGARVYGEISGYGSTIDPKAGSGRPPGLRKAIELALADAGVAPGEVDVVFADAAADPELDRQEAEAINAVFGTRGVPVTAPKTMTGRLYSGAAPLDLAAAFLAMKDGLIPPTVHIDPAAEYDLDLVTGEPRTAEVRTALVVARGYGGFNSAVVVRAA

SEQ ID NO: 30 - DNA, 267 bp, Streptomyces murayamaensis ATCC 21414
atggccaccacgttcaccctcgacgacctcaagcgcatcctccttgaggcagccggcgccgacgagggcgtcgacctggacggcgacattctggacaccgagttcgaggtcctgggatacgagtcgctcgccctgctggagaccggcggccgcatcgagcgcgagtacggcatctcgctggacgacgacgcgctgaccgacgcggtcaccccgcgcgccctcatcgaggtcgtcaacgcccagctgtccgccgcgtccgccgcctga

SEQ ID NO: 31 - protein, 88 aa, Streptomyces murayamaensis ATCC 21414
MATTFTLDDLKRILLEAAGADEGVDLDGDILDTEFEVLGYESLALLETGGRIEREYGISLDDDALTDAVTPRALIEVVNAQLSAASAA

SEQ ID NO: 32 - DNA, 786 bp, Streptomyces murayamaensis ATCC 21414
atgaccgagaacaccgcacgggtcgcgctggtcacgggtgccacgagcggcatcgggctctccgtcgcccggctgctcggctcgcagggccacaaggtcttcatcggcgcgcgcaacgccgacaacgtcgccgagacggtcaagcagctccagggcgagggcctggaggccgacggctcggcgctcgacgtcaccgacgccgccagcgtcaaggccttcgtccaggcggccgtcgaccgcttcggcaccgtcgacgtgctggtgaacaacgccggccgctccggtggcggcgtcaccgccgacatcgaggacgagctgtgggacgccgtcatcgacaccaacctgaacagcgtcttccgggtcacccgtgaggtcctgaacaccggtggcatgcgccacaaggaccgcggccggatcatcaacatcgcctccaccgcgggcaagcagggcgtggtgctcggcgccccgtactcggcctccaagcacggtgtggtcggcttcaccaaggccctgggcaacgagctcgcgcccaccggcatcacggtcaacgccgtctgccccggctacgtcgagacgccgatggcgcagcgcgtgcgccagggctacgcggccgcgtactccacctccgaggacgcgatcctggagaagttccagtccaagatcccgctcggccgctactccaccccggacgaggtcgccggtctggtcggctacctcgcctcggacacggccgcgtccatcaccgcgcaggcgctcaacgtctgcggcggcctcggcaacttctag

SEQ ID NO: 33 - protein, 261 aa, Streptomyces murayamaensis ATCC 21414
MTENTARVALVTGATSGIGLSVARLLGSQGHKVFIGARNADNVAETVKQLQGEGLEADGSALDVTDAASVKAFVQAAVDRFGTVDVLVNNAGRSGGGVTADIEDELWDAVIDTNLNSVFRVTREVLNTGGMRHKDRGRIINIASTAGKQGVVLGAPYSASKHGVVGFTKALGNELAPTGITVNAVCPGYVETPMAQRVRQGYAAAYSTSEDAILEKFQSKIPLGRYSTPDEVAGLVGYLASDTAASITAQALNVCGGLGNF

SEQ ID NO: 34 - DNA, 936 bp, Streptomyces murayamaensis ATCC 21414
atgacgacccgcgaggtcgagcacgagatcaccatcgaggcccccgccgccgccgtgtaccggctgctggcggaggtcaccaactggccgcggatcttcccgccgacgatctacgtcgaccaggtgggcgagcacgacaaccacgagcgcatccggatctgggccaccgccaacggcgaggccaagaactggacctcgcaccgtgagctcgaccccgaggcgctgcggatcaccttccgccaggaggtcaccacgccgccggtcgccgcgatgggcggcacctggatcatcgagaccctgggcgagaccacctcgcgggtccggctgctccacgactaccgggcgatcgacgacgaccccgaggggctggcctggatcgacgaggcggtcgacaagaacagccgctcggagctggccgcgctgaagcagaacgtcgaactggcccacgcgaccgaggaggtgacgttctcgttcaccgacaccgtcatcgtccagggctcgcccaaggacctgtacgacttcatcaacgaggcgaacctgtggtccgagcggctgccgcacgtggccgtcgtccggctcaccgaggacaccccggggctgcagaccctggagatggacacccgcgccaaggacggctcggtgcacaccaccaagtcgtaccgggtgaccttcccgcaccacaagatcgcgtacaagcaggtcacgctgcccgcgctgatgaccctgcacaccgggatctggacgttcgaggagacgcccgagggcacggccgcctcctcgcagcacaccgtcacgctcaacacggacaacatcgcgaagatcctcggccccgaggccaccgtcgcggacgcccgtgagtacgtgcacaccgcgctgtccaccaacagcacggcgacgctcaaccacgccaagacgtacgccgagtcgaagggctga

SEQ ID NO: 35 - protein, 311 aa, Streptomyces murayamaensis ATCC 21414
MTTREVEHEITIEAPAAAVYRLLAEVTNWPRIFPPTIYVDQVGEHDNHERIRIWATANGEAKNWTSHRELDPEALRITFRQEVTTPPVAAMGGTWIIETLGETTSRVRLLHDYRAIDDDPEGLAWIDEAVDKNSRSELAALKQNVELAHATEEVTFSFTDTVIVQGSPKDLYDFINEANLWSERLPHVAVVRLTEDTPGLQTLEMDTRAKDGSVHTTKSYRVTFPHHKIAYKQVTLPALMTLHTGIWTFEETPEGTAASSQHTVTLNTDNIAKILGPEATVADAREYVHTALSTNSTATLNHAKTYAESKG

SEQ ID NO: 36 - DNA, 1473 bp, Streptomyces murayamaensis ATCC 21414
atgaccccggaccgcctggacacacaggtcatcgtcgtcggcgccggccccgtcgggcttctgctcgccggtgagctgcgtcttggcggcgccgacgtggtcgtactggaacaacgggccacgcccaccacggagtcgagggcctccacgctgcacgcccgcaccatggagctccttgacagccgcggcctgctcgacctgttcgggacgccgccgaacgagccgcgcggccacttcggcggcatcccgatggacctcacgctgcccagccccttcccggggcagtggaagatgccccagacccggaccgaggcgctgctccaggagtgggcgctgtcgctgggcgcggacatccggcgcggccacgagctggtcgccgtgtccgacgagggcgacttcgtcgaggcccgggcggccgggccggagggcacggtcgtggtgcgcgggcggttcctcgtcgggtgcgacggcgaggagtcggccgtgcgccgcctgacgggcgccgagttcccgggcaacgacgccggccgcgagctgctgcgcgcggacgtggccggtgtcaccatcccgggccgccgcttcgagcggctgcccgccgggctcgccatcgcggcgacccgcgacggggtgacccgggtgatggtgcacgagttcggctcccaggccgaaccccgcaccggcgacccggagttcggcgagatcgcggcggtctggaagcgcgtcaccggcgaggacatcagcggcggaaccccgctgtgggcgaactcgttcggcgacgccaaccgccagctcacgcactaccgcgacggccggatcctgttcgccggcgacgcggcccaccggcagatgccgatcggcggccaggccctcaacctgggcctccaggacgccttcaacctgggctggaagctggctctgcacctcggcgagtcggcccccgagggcctgctcgacacgtaccacagcgagcggcacgaggtcggccggcgggtgctttccaacatcagggcacaggccatgctgctgctcggcggccaggaggtcgagccgctgcgcgcggtgctgaccgagctcctgccgtacgacgacgtccgggcgcacctcgccgggatgatcagcggcctcgacatccgttacgacgtgggcggccccgagcacccgctgctcggcgcacggctgccggacgccggtctcaccaccggcgaaggcccgctgagcaccgcccagttgctgcgcaccgcacgcggtgtgctcctcgacctgtccggtggcagtgcggtgctgtcggacgccgccggctgggcggaccgggtcaccgctctgcccgccgtgccggagaagggcggcgccctcgactcggtgggcgccgtcctggtccggcccgacggccatgtggcctgggccggcgccccggacaccgacggcgccgggctgcgggaggccctggagcgctggttcggcccctcgcactga

SEQ ID NO: 37 - protein, 490 aa, Streptomyces murayamaensis ATCC 21414
MTPDRLDTQVIVVGAGPVGLLLAGELRLGGADVVVLEQRATPTTESRASTLHARTMELLDSRGLLDLFGTPPNEPRGHFGGIPMDLTLPSPFPGQWKMPQTRTEALLQEWALSLGADIRRGHELVAVSDEGDFVEARAAGPEGTVVVRGRFLVGCDGEESAVRRLTGAEFPGNDAGRELLRADVAGVTIPGRRFERLPAGLAIAATRDGVTRVMVHEFGSQAEPRTGDPEFGEIAAVWKRVTGEDISGGTPLWANSFGDANRQLTHYRDGRILFAGDAAHRQMPIGGQALNLGLQDAFNLGWKLALHLGESAPEGLLDTYHSERHEVGRRVLSNIRAQAMLLLGGQEVEPLRAVLTELLPYDDVRAHLAGMISGLDIRYDVGGPEHPLLGARLPDAGLTTGEGPLSTAQLLRTARGVLLDLSGGSAVLSDAAGWADRVTALPAVPEKGGALDSVGAVLVRPDGHVAWAGAPDTDGAGLREALERWFGPSH

SEQ ID NO: 38 - DNA, 1503 bp, Streptomyces murayamaensis ATCC 21414
atggaggggacagccgtggacaccgatgtgatcatcgtcggcgcgggtccgaccggcctcatgctcgccggggaactgcgcctcggcggggcggacgtcgtcgtcgtcgaacggctgacgaagcccaccggccagtcccggggcctgggcttcaccgcccgcgccatggagatcttcgaccagcgcgggctgctgccccggttcggccagggcgagacgctggagatcagcccgctcggtcacttcggcggtgtgcagttcgactacaccgtcctggagggcgcccacttcggggcgcgcggcattccccagaacatcaccgagacggtccttgaggagtgggcgaccgagctcggcgtggacatccggcgcggctgggacttcctggagatagccgacggctacctcgacggcgacagcgtcgagatcaaggtgcagacgcccaactcggtacggaagctgcgcgcttcctacctcgtgggcgccgacggcggccgcagcgtggtgcgcgaggcggccgggttcgacttcccgggcacctcggccacccgggcgatgttcctggccgatgtgaccggctgcaacctcaagccgcgcttcctcggtgagcggctgaacaacggcatggtgatggcggccccgctcgccgagggcgtcgaccgcatcatcgtctgcccggacggcacgcccgcgcgcgccagcggcgacacggtcagcttcgaggaggtcgccgccgcctggcagtcgatcaccggcgaggacatctcgcacggcggcgccgagtgggtcagcttcttcagcgacgccacccgccaggcctccgagtaccggcgcggccgggtcctgctggtcggcgacgccgcccacatccacctcccggccggcggccagggcctgagcaccggcgtccaggacgcggccaacctcggctggaagctggccgcggcggtcgccgggaccgcgcccgaggggctgctcgacacgtaccacggcgagcgccaccccgtgggtgcccggctgctgatgaacacccgcgcccagggcatggtgttcctcggcggacccgaggccgagccgctgcgccagctcttcggcgagctcatccagtacgacgacgtgaagcgccatctcgccgggatcgtcagtggtctggacatccggtacgagctgggtgacgcgcacccgctggtggggcgccggattccgcctcggcggctggtgggggcggcgggggagaccagcaccgtcgcgctgctgcacgcggcgcggggtgtgctgctcgacttcgccgacgacgcggcggtgcgggacgcggccgccgggtggtcggggcgcgtcgacgtcgtcacggcggcgccgaagccggtcgacggcggtaccgatccgctcgcgggtgcggctgccgtgctcgtacggcccgatggatatgtggcgtgggccgcggacacggccgaaggccttgctccggctcttgagcgctggttcggtccggccggggtgtga

SEQ ID NO: 39 - protein, 500 aa, Streptomyces murayamaensis ATCC 21414
MEGTAVDTDVIIVGAGPTGLMLAGELRLGGADVVVVERLTKPTGQSRGLGFTARAMEIFDQRGLLPRFGQGETLEISPLGHFGGVQFDYTVLEGAHFGARGIPQNITETVLEEWATELGVDIRRGWDFLEIADGYLDGDSVEIKVQTPNSVRKLRASYLVGADGGRSVVREAAGFDFPGTSATRAMFLADVTGCNLKPRFLGERLNNGMVMAAPLAEGVDRIIVCPDGTPARASGDTVSFEEVAAAWQSITGEDISHGGAEWVSFFSDATRQASEYRRGRVLLVGDAAHIHLPAGGQGLSTGVQDAANLGWKLAAAVAGTAPEGLLDTYHGERHPVGARLLMNTRAQGMVFLGGPEAEPLRQLFGELIQYDDVKRHLAGIVSGLDIRYELGDAHPLVGRRIPPRRLVGAAGETSTVALLHAARGVLLDFADDAAVRDAAAGWSGRVDVVTAAPKPVDGGTDPLAGAAAVLVRPDGYVAWAADTAEGLAPALERWFGPAGV

SEQ ID NO: 40 - DNA, 879 bp, Streptomyces murayamaensis ATCC 21414
atggaaagcacgctcgcaccgggcgcggtctcccagggcgttcgcaggatcaccctggacgccgggggagtcacgctgtccgcgctgctgtgcgagccggaagggaccccccgcgccaccgtcgtcgccgtgcacggcggcgggatgagcgccgggtacttcgacggtcaggcgcaccccgagctgtccctgctcaccctcggcgcccggctcggctacaccgtgctcgcggtggaccggcccggctacggccgttccgccgcccagctgccggacgggctcaccgtcgccgagcagaccgaggtgctgcgggccgggatcgacgacttcacctccaagtacccgacgggcgcgggggtgttgctggtcgcccactccttcggcggcaagctcgccctgtcggccgccgcgcactgcaccggcgacggcctgctcggcatcgacatctccggctgcggccaccgctacgccgtcaccccgggcgtgctgcgcaagggcctcaagcacatcgcccggcactggggcccgctgcggctctacccgccggacaccttccgcagcagcggctccctggtggcgccgatgccggagcgcgaggcgagtgaactcaagcgctggcccgagctgttcgcggccctcgcgccgcgcgtgcggatcccggtccggctcaccttcgccgagcacgagggctggtggctgcacggcgagcaggacctcgccgacctcgccgcccagctgaccgcctcgccccgtatcgtcgtcgaccgccagccggacgccggtcacaacatcagcctcggctgggcggcccgctcctaccacctgcgcaccctcgcgttcctggaggactgcatcacgcgggcgggacgcgatgggtga

SEQ ID NO: 41 - protein, 292 aa, Streptomyces murayamaensis ATCC 21414
MESTLAPGAVSQGVRRITLDAGGVTLSALLCEPEGTPRATVVAVHGGGMSAGYFDGQAHPELSLLTLGARLGYTVLAVDRPGYGRSAAQLPDGLTVAEQTEVLRAGIDDFTSKYPTGAGVLLVAHSFGGKLALSAAAHCTGDGLLGIDISGCGHRYAVTPGVLRKGLKHIARHWGPLRLYPPDTFRSSGSLVAPMPEREASELKRWPELFAALAPRVRIPVRLTFAEHEGWWLHGEQDLADLAAQLTASPRIVVDRQPDAGHNISLGWAARSYHLRTLAFLEDCITRAGRDG

SEQ ID NO: 42 - DNA, 1281 bp, Streptomyces murayamaensis ATCC 21414
atgaccagcactctggcaacccctttccgttccctgtccgtacggaacttccggctgttcgcggccgggcaggtggtctccgtcgcgggcacctggatgatggtcgtggcccaggactggatcgtcctgagcctggccgacaactccggtacggcgctgggcgtggtgaccgcgctgcagttcaccccgctgctgctgctcaccctgtacggcgggcgcctcgccgaccgctacgacaagcgcttcctgctgacctgtgccaatctcgcgtccggcgcgctggctctggtgctcgcgctgctcgcgttcgcggacgcggtgcagctgtggcacatctggctgtgcgcgttcggcctcgggatggtgaacgccgtcgaggtgccgacccggatggcgttcgtcagcgagctggtcggccccgaactgctgcccaacgcctccgcattgagcgccgcgtacttcaacaccgcccgggtcgtcggcccggcgctggccgggctgctcatcaccggcttcggcaccggctgggtcatgctgttcaactccgtcagctatctggccacggtggccgggctgcggatgatgcggccggacgaactgctgcgcggcgcacggcaggacacccgtccccgggtgatcgacgggctgcggtacatccgcagccgccccgatctgaagctgccgctcgccctgatcggggtgatctcgctcgtcgggctcaacttccagctgacgctgccgctgtatgccaaaacggttttccacgccgacgcggcctcgttcgggctgctgaccaccggcttcgcggcgggctccctggtcgccgcgttcgtcaccacggcgcgccgcggccgcccctccagccgtctggtggtcgcctcggcgatcgcgttcgcggccttggagacggtggcgggctgggcgcccaacttcgcctcggcgatcgtgctgctctcgctcaccggcggggcgaccatctacttcgtccaggcggccaaccatcgcgtccagctcggcagcgacccgcagtaccggggccgggtgatggcgctctacacgctcatcgtccagggctccaccccgctgggatcgctcctcatcggctggctcgccgaacacctgggcgcccgctcggggttctacgtgggcggcctggtctcgctggcggccgccctgacggcgctggccttcgaccggcgtacgggacaggaggcgtccgacgacgtgacgacggcgacgaaggccgcgcccgagggcgagcccgaggcggtgagccggtga

SEQ ID NO: 43 - protein, 426 aa, Streptomyces murayamaensis ATCC 21414
MTSTLATPFRSLSVRNFRLFAAGQVVSVAGTWMMVVAQDWIVLSLADNSGTALGVVTALQFTPLLLLTLYGGRLADRYDKRFLLTCANLASGALALVLALLAFADAVQLWHIWLCAFGLGMVNAVEVPTRMAFVSELVGPELLPNASALSAAYFNTARVVGPALAGLLITGFGTGWVMLFNSVSYLATVAGLRMMRPDELLRGARQDTRPRVIDGLRYIRSRPDLKLPLALIGVISLVGLNFQLTLPLYAKTVFHADAASFGLLTTGFAAGSLVAAFVTTARRGRPSSRLVVASAIAFAALETVAGWAPNFASAIVLLSLTGGATIYFVQAANHRVQLGSDPQYRGRVMALYTLIVQGSTPLGSLLIGWLAEHLGARSGFYVGGLVSLAAALTALAFDRRTGQEASDDVTTATKAAPEGEPEAVSR

SEQ ID NO: 44 - DNA, 576 bp, Streptomyces murayamaensis ATCC 21414
gtgtgcccgctgatgcaggccctggacgagctggaggcggcccgcgcggccaccttcgtccacgacagggaccgccgccagtacgtggcggcgcacgccaccctgcggcgcgtgctcgccgagtacaccgggcacgagcccagccgggtgccgctcggccgggccgaagggccctacgggaagccgcagttgatcggttcgccggtcccgctgcacttcaacctctcgcacagccacggcctgatcgccatcggggtcgcggcggacccggtgggcgtcgacgtccagcgcgtcccgtcgcccgaggcggtcgaggtggtcctgcccaggctgcatccgcgcgagcgcgaggaactgcgcgctctgcccgcatcggagcgcccggaggcgttcgcgcggctgtggacccgcaaggaggcctacctcaagggcctgggcaccggcctcacccgctcgcccgcggcggactatctgggcgagacggccgcggcccgcccggccggctggacggtgcgcaacgtgccggtacagccgggctacgcggccgcggccgcgctccgccaccacccgacctga

SEQ ID NO: 45 - protein, 191 aa, Streptomyces murayamaensis ATCC 21414
MCPLMQALDELEAARAATFVHDRDRRQYVAAHATLRRVLAEYTGHEPSRVPLGRAEGPYGKPQLIGSPVPLHFNLSHSHGLIAIGVAADPVGVDVQRVPSPEAVEVVLPRLHPREREELRALPASERPEAFARLWTRKEAYLKGLGTGLTRSPAADYLGETAAARPAGWTVRNVPVQPGYAAAAALRHHPT

SEQ ID NO: 46 - DNA, 1581 bp, Streptomyces murayamaensis ATCC 21414
atgccgaccacgccgaccacacagtcctccgccgaggtctccgaccggctggacgaactcagcgaacgcaaggaacaggccgtacgcggtcccagcgacaaggcgaccgaggcgcagcacgccaagggcaagctgaccgcacgcgagcggatcgaactcctcctggacaagggcagcttcaccgaggtcgagcagctgcggcggcaccgcgccaccgggttcggcctggaggccaagaagccgtacacggacggtgtcatcaccggctggggcacggtcgagggccgtacggtcttcgtctacgcccatgacttccgcatcttcggcggcgcgctcggcgaggcccacgcgaccaagatccacaagatcatggacatggcgctggcggcgggcgcgccgctggtctcgctgaacgacggcgcgggcgcccggatccaggagggcgtctcggcgctcgccggttacggcggcatcttccagcgcaacacgcgggcctcgggtgtcatcccgcagatctccgtgatgctcggcccgtgcgcgggcggcgcggcgtactcgccggcgctgaccgacttcgtgttcatggtccgcgagacctcgcagatgttcatcaccggcccggacgtcgtccaggccgtgacgggcgaggagatcagccagaacggactcggcggcgccgatgtgcacgccgggacctcgggcgtggcgcacttcgcgtacgacgacgaggagagctgcctcgccgaggtgcgctatctgctctccctgctgccgtccaacaaccgggagatgccgccgctggcgcagacctcggacccggtggaccgcgagggcaccgccctgctcgatctggtgccggccgacggcaaccgctcgtacgacgtgcgcggggtgatcgaggagctcgtcgacgacggcgagtacatggagatccacgccaactgggcgcccaacctggtggtggccctggcccggctggacggccatgtcgtcggcgtcgtcgccaaccagccgtccgccatggccggcgtcctggacatcaaggcgagcgagaagggcgcccggttcgtccagttctgcgactccttcagcatcccgctgatcaccctcgtcgacgtgcccgggttcctgccgggcgtcgaccaggagcacgacggcatcatccggcgcggcgcgaagctgctctacgcctactgcaacgcgaccgtgcctcgtatctcggtggtgctgcgcaaggcgtacggcggtgcctacatcgtgatggactcgcgttccatcggagccgacctgtcgttcgcctggcccaccaacgagatcgcggtgatgggcgccgagggcgcggcgaacgtggtgttccggcgggagatcgccgcggccgaggacccggacgcgatgcgcaagcagaagatcgacgagtacaagaacgagctggtgcacccctacttcgcggccgagcgcggtctggtcgacgacgtcatcgacccgcgcgagacccgctcggtgctgtgccgctcggtcacgatgctcatcgccaaggacgccgagctgccccgccgcaagcacggcaacccgccccagtag

SEQ ID NO: 47 - protein, 526 aa, Streptomyces murayamaensis ATCC 21414
MPTTPTTQSSAEVSDRLDELSERKEQAVRGPSDKATEAQHAKGKLTARERIELLLDKGSFTEVEQLRRHRATGFGLEAKKPYTDGVITGWGTVEGRTVFVYAHDFRIFGGALGEAHATKIHKIMDMALAAGAPLVSLNDGAGARIQEGVSALAGYGGIFQRNTRASGVIPQISVMLGPCAGGAAYSPALTDFVFMVRETSQMFITGPDVVQAVTGEEISQNGLGGADVHAGTSGVAHFAYDDEESCLAEVRYLLSLLPSNNREMPPLAQTSDPVDREGTALLDLVPADGNRSYDVRGVIEELVDDGEYMEIHANWAPNLVVALARLDGHVVGVVANQPSAMAGVLDIKASEKGARFVQFCDSFSIPLITLVDVPGFLPGVDQEHDGIIRRGAKLLYAYCNATVPRISVVLRKAYGGAYIVMDSRSIGADLSFAWPTNEIAVMGAEGAANVVFRREIAAAEDPDAMRKQKIDEYKNELVHPYFAAERGLVDDVIDPRETRSVLCRSVTMLIAKDAELPRRKHGNPPQ

SEQ ID NO: 48 - DNA, 306 bp, Streptomyces murayamaensis ATCC 21414
gtgcacgacatgagcgagatgacccagttcaccgagcccgccgagcccgccgccgagtccgccgggatgaccctggagcactgccgcgagctgttgcgggtcgagcggggcaaccccgatccggaggaactcgcggcgctggcggcgctgttcttcgcccacttctccgcgatcgaggcgcggcgggaggccgcccgtgtcctgatcccgcggcagcggcgctccgcgagctggcgccgcaccgagcgggcacccggcttcgacggcccgcgcacctggcgcgcgggcggtcccgcactcgtttga

SEQ ID NO: 49 - protein, 101 aa, Streptomyces murayamaensis ATCC 21414
VHDMSEMTQFTEPAEPAAESAGMTLEHCRELLRVERGNPDPEELAALAALFFAHFSAIEARREAARVLIPRQRRSASWRRTERAPGFDGPRTWRAGGPALV

SEQ ID NO: 50 - DNA, 783 bp, Streptomyces murayamaensis ATCC 21414
atgactcagagttcgtcagaggcagtgctggagcctcatatacctgtccagcgtggtcccggtggaccacacctgatcgaagagggcgtgtcggaagcggtgcgcagggtggccggcctctcgcggggccggcggatcctcgtggtcgacagcgacgtcgacggcgcggagtccctggtgtgccggctgcgcaggcacggccacgaggccatcggcgtgaagagcggtagcaccgcgctgcaggcgtacgaggacgtggaccttgtcctcctcgacctcgaactcccggacctggacgggctggaggtgtgccgggccatccgctccgtgagcggcatccctgtgatcatcgtcaccgcccggggctccgagctcgactgtgtgctcggcctacaggccggtgcagacgactatgtggtcaagccctatggcttccgggaattaatggcacggatcgaagccgtcatgcgtcgcgccaggttccaaccgcctgttgccagagagatcttgcacgggcggttgcgcattgacgtgagctcccgcgaggtgagcctggacggccgcgaggtggggctgacccgcaaggaattcgatctgctctgcctgctcgcgtcccatccggacacggtcattccgcgaaagcgcctgctccagcaggtctggggggactcctggtcccgccgtactgtcgacacccatgtcagcagccttcgcggaaaactcggcgacagcggctggatcattactgtgcgcggggtcggtttcaagctgggcaacgggtga

SEQ ID NO: 51 - protein, 260 aa, Streptomyces murayamaensis ATCC 21414
MTQSSSEAVLEPHIPVQRGPGGPHLIEEGVSEAVRRVAGLSRGRRILVVDSDVDGAESLVCRLRRHGHEAIGVKSGSTALQAYEDVDLVLLDLELPDLDGLEVCRAIRSVSGIPVIIVTARGSELDCVLGLQAGADDYVVKPYGFRELMARIEAVMRRARFQPPVAREILHGRLRIDVSSREVSLDGREVGLTRKEFDLLCLLASHPDTVIPRKRLLQQVWGDSWSRRTVDTHVSSLRGKLGDSGWIITVRGVGFKLGNG

SEQ ID NO: 52 - DNA, 405 bp, Streptomyces murayamaensis ATCC 21414
gtgacctggacgactgagcgggtggagggcgccgacctggacctggaggcggtcctcgacgtctaccgcagctccgggctcggcgagcgccgtcccatcgaggacgtggagcggttcgccgccatggtccgcaacgccaacctcgtggtggtggcgcgggacgcggagggcaggctcatcggcatcgcccgcagcatctccgacttctcctacgcgacgtacctctcggacatcgcggtgagcggcgactaccagcgctcgggcatcggccgcgcgctcatcgacgccacgcagaaggaggccccgcaggcgaagatcattctcctgtcggcgccggcggcggtggagtactacccgcacatcggcttcacccagcacaactccgcctgggtgctcaacccgtag

SEQ ID NO: 53 - protein, 134 aa, Streptomyces murayamaensis ATCC 21414
VTWTTERVEGADLDLEAVLDVYRSSGLGERRPIEDVERFAAMVRNANLVVVARDAEGRLIGIARSISDFSYATYLSDIAVSGDYQRSGIGRALIDATQKEAPQAKIILLSAPAAVEYYPHIGFTQHNSAWVLNP

SEQ ID NO: 54 - DNA, 1299 bp, Streptomyces murayamaensis ATCC 21414
gtgctggcccacatgattccccgctataccctgcccgcgatggcggacatcttctcggaccaggcgcggtacgcgacctgggtccgggtggagatcctggcttccgaggcgcaggcgcgcctgggacgggtgcccgaggacgcggtcgaggacatgcggcgggccaaggtcccgacgcccgagcgggtgcaggagatcgagcgcgagcgcgaccacgaagtgctctcgttcctcgccgcgtactgcgaggacatcccggacgagtcggcccgctgggtccacctcggcatgaccagctacgacctcgtcgacacctcgctgggctacaacctggcccgcgccaccgacctggtgatcgcggccgcggtcgagctgcgcaaggtcctggtcgaacgggccctggagcactgggagacggtcatcgtcggccgcacccacggcgtccacgccgagccgacgtccttcggccacaagctggcgcagttcgcgttcgcggtggaccgctcgatcacccggctgcgcgcggcgcgcaaggccgtggcggtgggcacgatctccggctcggtcggcacgtacgcgctgatcgacccctccgtcgaggcgtacgtctgcgaggagctggacctgggggtggagccggccccgagccaggtcgtcgcccgcgaccggcacgcccagctgatccaggccgtcgccgtgctcggcgcgagcgtcgagcagatcgccctggagctgcggctgctgcagcgcaccgaggtccgcgaggtcgaggagcagcgcacctcggcgtaccagggctccagcgccatgccccacaagcgcaacccgaccaccagcgagcgtctgacgggtctggcccggctgctgcgcggttacgcgaccacggccctggagaacgtggcgctgtggcacgagcgggatctggcccaccagtcggtggagcgggtgatcctgccggacgcgctgtcggtggggcacttccaggccacgatggcggccgacctggtccgtaacctcaaggtgttcccggagcggatgcgggcggggatcgaccagaccgacggtcttgtgttcagctctgccgtactcgccgatctgctggccgacggggtggagcgggagaaggcctaccgggccgtccagtccgccgccaaccgcaccatcgcgaccggcgagcacttcggggacacgctgcggcaggagggcatggacatcggccagctccgcccggagcgtttcctcgtcaaccacggtgtgattctcaagcgattggagcagcttcgtgacctggacgactga

SEQ ID NO: 55 - protein, 432 aa, Streptomyces murayamaensis ATCC 21414
VLAHMIPRYTLPAMADIFSDQARYATWVRVEILASEAQARLGRVPEDAVEDMRRAKVPTPERVQEIERERDHEVLSFLAAYCEDIPDESARWVHLGMTSYDLVDTSLGYNLARATDLVIAAAVELRKVLVERALEHWETVIVGRTHGVHAEPTSFGHKLAQFAFAVDRSITRLRAARKAVAVGTISGSVGTYALIDPSVEAYVCEELDLGVEPAPSQVVARDRHAQLIQAVAVLGASVEQIALELRLLQRTEVREVEEQRTSAYQGSSAMPHKRNPTTSERLTGLARLLRGYATTALENVALWHERDLAHQSVERVILPDALSVGHFQATMAADLVRNLKVFPERMRAGIDQTDGLVFSSAVLADLLADGVEREKAYRAVQSAANRTIATGEHFGDTLRQEGMDIGQLRPERFLVNHGVILKRLEQLRDLDD

SEQ ID NO: 56 - DNA, 1503 bp, Streptomyces murayamaensis ATCC 21414
atgtactcccggtcgtggtccagccccttctccgaggcgggcggcacaggacgtcccgcgttcgtcaccgagttcggcctgtggaccgacgaacaggccgctgcggccgagcagatcgaggcctcgctcgacgagatcgacctggtccgcctcgccttcgccgacccgcacgggctggcccgctccaagacgctgaccgtggacgcgttccgctcggtcctgcgcaacggcatggacttcagctcgggcccgttcatcttcgacaccggccacgcggccgccctggacttcctcgccgacccgggcgtcggcgtcgacgagatcgcgggcgcgggcagcttcgtgctggtcccggacccgctgacgttccaggtgctcccccacgagggaccgcgcaccgcgtgggtgctcggtgacgagtatctgcgcgacggctccccgcacccgctctccgcgcggaacgtgctgcgccaggtcgtcgcccggtacgcggcccgcgacctcaccccggtgctcggcctggaggtcgagtggtacctgacccgcaagctcgcgggtccgcccgggaacgcgggcaacggcttcggcctccagggcgcggcccccaaggtcgaggcgatgaactcgggctaccagttcaacctggacgccaactacgactcggtggcccacttcaccagcccgctggcgatgaagctgctcgaactcggcctgccgctgcgctcgatcgagcacgagtcgggcccgggccagatcgagacgaccttcaacccgatgcacgcgctggacaccgccgacgcgatgctcctgttccgcaccgtggtcaagcagaccgccgcgcgccagggctaccacgcctccttcatggcgctgccccgcgtcgacagcttcgacccgtgcggctggcatctgcaccagtcggtgatggacagcacgaacgggcgcaacctcttcgcggccgacggcggcggcatctcggaccagggcaaggcgtacatcgacgggctgctctcccgcgcccgcgacctgtgtctgctctcggtccccaccgtcaacggctaccgccgcatgggcgcggacttctcgctctcgccgacccgtctcggctggagctacgaggaccgcagcgcgatgatccgggtggtcggcggcggcgccggcacgcacatcgagaaccgggtgggcgagcccaccgccaacccgtacctcaacatcgccgcccagctctccgccgggttcgacggcatggccaccgaggtcggcgcgagcacgcgggagagcggcgaggagtcatacgagacgctgccgcaggacctcggcgaggcgctggaggccttccgggccggtcaggccgccgaactgctcggcaagccgctggccgccaccctggccaagctgaaggagagcgagctgtcccgctacgaggcctggcgcgcggccgagcggcccgccgacggccaggtcaccgagtgggagcagcgcgaatacttcgaggccttctga

SEQ ID NO: 57 - protein, 500 aa, Streptomyces murayamaensis ATCC 21414
MYSRSWSSPFSEAGGTGRPAFVTEFGLWTDEQAAAAEQIEASLDEIDLVRLAFADPHGLARSKTLTVDAFRSVLRNGMDFSSGPFIFDTGHAAALDFLADPGVGVDEIAGAGSFVLVPDPLTFQVLPHEGPRTAWVLGDEYLRDGSPHPLSARNVLRQVVARYAARDLTPVLGLEVEWYLTRKLAGPPGNAGNGFGLQGAAPKVEAMNSGYQFNLDANYDSVAHFTSPLAMKLLELGLPLRSIEHESGPGQIETTFNPMHALDTADAMLLFRTVVKQTAARQGYHASFMALPRVDSFDPCGWHLHQSVMDSTNGRNLFAADGGGISDQGKAYIDGLLSRARDLCLLSVPTVNGYRRMGADFSLSPTRLGWSYEDRSAMIRVVGGGAGTHIENRVGEPTANPYLNIAAQLSAGFDGMATEVGASTRESGEESYETLPQDLGEALEAFRAGQAAELLGKPLAATLAKLKESELSRYEAWRAAERPADGQVTEWEQREYFEAF

SEQ ID NO: 58 - DNA, 1386 bp, Streptomyces murayamaensis ATCC 21414
ttgacctcagcgtccattggcgacatacgcgacctcctcgcccggggagagctgtccgccgccgaccatgtgcagtcggtcctcaccgcgatccagaagaccgacatcgagctcggcgccttcgtctcggtcgcgggcgacgaggccgtgcgggaggccgaactggccgacgcccggatccgcgaggagggcccggccgtcttcgaccggcagccgctgctcgggatcacggtctcggtgaaggacctcatccagaccggggacctgcccactgcccgtgggtccctcctggagaaccggcggccccgggccgacgcgccctcggtcgcgcggctgcgggccgccggggccatcgtcatcggcaagaccacgacgtcggagtacgggtggagcgcctccacggtgagccgggtggccccgcccacccgtaacccgtgggatctggaactctccgccggcggctccagcggcggcgccgcggccgcggtcgcggcggggctcggctcgggggcgctcggcaccgacggcgcgggctcgatccgtatcccgtcggcgttctgcggtgtggtcggctacaagccgtcgttcgccaaggtgccgtatgtgcccgcctgcgccgaccggctctcccaccaggggccgatcgcgcgcaccgtgccggacgtcatcgagctcgcctcggtgatcaccggcgggcatccgcaggacccggactcgatgctcggcgtgcatgaactgccccgccagcggcggcggttgcgcatcggctggatcgagttcccgggcacctcgccggaggtccgccgggtcagcgagcagggtctggacgcgctcgccgcgcaggggcaccgcgtcgagcggatcgaggtgccgttccgcgacccgtatccggcgctcgtcgacatcctcgccgcgagcgacgcggcgggtacctcgcccgccgacgaggagtggtgcgacccgggccgcctcgcgatcgtgcggcacggccgcacgctcagcgcggccaccgtgatgcgggccgaggaggtgcgtctggcgctgcgcaccacactgcaccagatcttcgaccggtacgacctgctcgcgatggccaccgtgcccatcgagccgatcgatccccacgcgatcggccctgactgggccagccgtccggaggacctgctctggctggcgtggacacccgccgcgtatcccttcaatatgactggccagccggccgtttcgctcccggccggactcacccgcgccggtctcccggtggggctccaactcgtgggccccttcggcgcggacgatctggtcctgtccgccgcacgccgtctggaggcggacctcgggccgctgccggccgcaccggaccgagtaaccgaaaggatcctgtaa

SEQ ID NO: 59 - protein, 461 aa, Streptomyces murayamaensis ATCC 21414
MTSASIGDIRDLLARGELSAADHVQSVLTAIQKTDIELGAFVSVAGDEAVREAELADARIREEGPAVFDRQPLLGITVSVKDLIQTGDLPTARGSLLENRRPRADAPSVARLRAAGAIVIGKTTTSEYGWSASTVSRVAPPTRNPWDLELSAGGSSGGAAAAVAAGLGSGALGTDGAGSIRIPSAFCGVVGYKPSFAKVPYVPACADRLSHQGPIARTVPDVIELASVITGGHPQDPDSMLGVHELPRQRRRLRIGWIEFPGTSPEVRRVSEQGLDALAAQGHRVERIEVPFRDPYPALVDILAASDAAGTSPADEEWCDPGRLAIVRHGRTLSAATVMRAEEVRLALRTTLHQIFDRYDLLAMATVPIEPIDPHAIGPDWASRPEDLLWLAWTPAAYPFNMTGQPAVSLPAGLTRAGLPVGLQLVGPFGADDLVLSAARRLEADLGPLPAAPDRVTERIL

SEQ ID NO: 60 - DNA, 2058 bp, Streptomyces murayamaensis ATCC 21414
atgtcgccaatggacgctgaggtcaacggagcagctcctcgtcagaacggggtgggccgccggtcacgagacctgctgcggctgcttggcgcgcgccgccgcggcctggaccgacttgaggtgccactcggagtcgaaggcggtgacctcgatctcctcgcccgcggccaccagcgcggcggcggcgctcatggcgccggcgatgaaccccgaggtgcggtgcgcggcgagcaggtcggactcgtcgaacggggtgccgtcgcagcgcagcagacgcaggtccatgaacccgttgaacctctggcccagcacctcgaccgactgcgggctgcggatgctgaaccgcaccttcgacgccacatggccgtggtggtgctggtcggagtagaaggcgacgtcggggatggtcggcgggatgaagtggtcgcgctccaccacggcgggcaccttgggcaggttcccggcgatgaagtgccaggagttgtactgcatgcgggacgacatcgcccaggcgttgtcggccagttgcagcaccgagtcctcgtagtggcgcttggccgacggggcgggcaccacgcagcagaagaagtcggtgatgtcccacttggtgatctcggcgtagctctgctcccgcagggcgcgcatcagttcggggatcgaacgcatgccccggctcatggcgaagtcggcctcgaagacctccgtcgccccggccaccgtctcgtacaccagggcctcaagtccggtaccgaactcgccgtacggccgggcgagccaggagggggacgcggccaggctcgcgcggagctcggcgaaggtggcgccggtgaccgggagacgctcgctcagcagggcggcgagctccgcctcgcgctgctgcgacttctccccgtacggaccggccaggcgctcgatcttgtgcatcagcgcgccgtggatctcgcggtagagctgggcgttggggatgacatggcccatgcggcgctcgcgcagcgccatggcgagggcgttcagcgcctcgacgcggtgcgtgggagcgggcgggacgagctcgccgccggggaccgcgttgtagttgccgcgggtgcgctccagcatgtacgcgatgtgatccagcgtcagctgggtgccgttggcctcctcgatgcgcccggcgcccgcggagcgcaccagcagcgtgaggcagacgaccatcaccgcgtcgtggtccgaccactgctccatggagacgcccatcagacggctgaacgtctcgttggggtggcccgcccacagcgtcttgccgatgacgttgttggtctcgcggaagttggtgtagagcttcccctcctggtgcagcacgaagggggcggtccgcagcgcggactcccgcagcatctccaggagttcgtcacgggcctgggtgccgagcgccgccagccactggcggcagtcgacgacctggtcggcgaagcgggcggccgcgcggtcgacctcgctctcgtagtcgagcaggtcctgcgccgaccacggcacgctcatgacgccctcgaccagcttctcctcgtcctccaacggcccctgcgagggcacgcggacgacgatgcggccggtcgtggtcaggcccagctcgccgaggaacgcggggcgctccttgacgtcgcgcacgggcgtcggcaggttctcctcgcgccacgagatgccgcagagcaccttgagcgcggcctcgtcggcggtgcgcagggcgcgcagctggacctcggcggggacgtggccgtccagggtgagcagggcgttcaccgcgccgcgcaggtccggggccgcggtcgcgcgctcccaggcgcgcttgagcagagtggggaaaccggcctcctcgtctttgaggcggtccggcttcggcgtggccccggccaccgcgccctggcggctcggccggcggctctgccgcttcttggcgttgatggcccggcgggaggtgccgttgcgctcggcggaacttgtcatgacacaccctcagcggtaagcccgatgtgcttccacatctcgtcggtcgtgggcgtccagtcctcgtcgacgtag

SEQ ID NO: 61 - protein, 685 aa, Streptomyces murayamaensis ATCC 21414
MSPMDAEVNGAAPRQNGVGRRSRDLLRLLGARRRGLDRLEVPLGVEGGDLDLLARGHQRGGGAHGAGDEPRGAVRGEQVGLVERGAVAAQQTQVHEPVEPLAQHLDRLRAADAEPHLRRHMAVVVLVGVEGDVGDGRRDEVVALHHGGHLGQVPGDEVPGVVLHAGRHRPGVVGQLQHRVLVVALGRRGGHHAAEEVGDVPLGDLGVALLPQGAHQFGDRTHAPAHGEVGLEDLRRPGHRLVHQGLKSGTELAVRPGEPGGGRGQARAELGEGGAGDRETLAQQGGELRLALLRLLPVRTGQALDLVHQRAVDLAVELGVGDDMAHAALAQRHGEGVQRLDAVRGSGRDELAAGDRVVVAAGALQHVRDVIQRQLGAVGLLDAPGARGAHQQREADDHHRVVVRPLLHGDAHQTAERLVGVARPQRLADDVVGLAEVGVELPLLVQHEGGGPQRGLPQHLQEFVTGLGAERRQPLAAVDDLVGEAGGRAVDLALVVEQVLRRPRHAHDALDQLLLVLQRPLRGHADDDAAGRGQAQLAEERGALLDVAHGRRQVLLAPRDAAEHLERGLVGGAQGAQLDLGGDVAVQGEQGVHRAAQVRGRGRALPGALEQSGETGLLVFEAVRLRRGPGHRALAARPAALPLLGVDGPAGGAVALGGTCHDTPSAVSPMCFHISSVVGVQSSST

SEQ ID NO: 62 - DNA, 402 bp, Streptomyces murayamaensis ATCC 21414
atgctctcttaccgtggcacgagcgaaacaggacaagaggggggccgaatgcccacgaacacttcggacgactcgctggacgagacagtggaaggctcggtgtccgggcgcgacaagctgatcgccgagcggacccgcagcgaaacgtggaagaagccgccacgccgtatcgagcgcgcggagtgcatcacctgcgacacctgcctgcgtgcctgcccgcccgagttcaacgcgatcttcgacaacggactcgacgtcgtcatcatccccgaactgtgctccggctgccccaagtgcgtcctggagtgcccggtcgactgcatctacgtcgacgaggactggacgcccacgaccgacgagatgtggaagcacatcgggcttaccgctgagggtgtgtcatga

SEQ ID NO: 63 - protein, 133 aa, Streptomyces murayamaensis ATCC 21414
MLSYRGTSETGQEGGRMPTNTSDDSLDETVEGSVSGRDKLIAERTRSETWKKPPRRIERAECITCDTCLRACPPEFNAIFDNGLDVVIIPELCSGCPKCVLECPVDCIYVDEDWTPTTDEMWKHIGLTAEGVS

SEQ ID NO: 64 - DNA, 642 bp, Streptomyces murayamaensis ATCC 21414
ttggatctggccgacccggtcccggccgaactggccgccaaactcgcctcgatgggccccgcgcacgaggaggtgctggcggactcggtgggcctcgccctccttgtcgtcctggagacgctggacccggccgagcggatggcgttcgtcctgcacgatctgttcggcctcccctatgacgaggtggcgccgatcgtggggaccggcgccgaggaggcgtgcgagctggccgaccgggcccgggcgcgggtgcggcgggtcgatccgctgccggacgacggtacgacgcggctgcgccggatcgtcgacgcgttcctgaccgcctcgcgcggcggcgacttcgccaccctgaccgcgctgctcgccccggacgtggtgctccgcgccgacccggaggcggtggcggtgggggcgccgacggaggtgcggggcgcgggctcggtggcggacgcgttctcggggcgcgccaagtacgccaagctggcgctggtcgacggcgtcgtcggggcggtgtgggcgccgcgcgggcggccgagggtcgtcttcgggttcaccgtcgtggacgagaagatcaccgggatcgacatgcgggccgcgcccgagcggctggaccggctcgatctgacgatcctgaacgactga

SEQ ID NO: 65 - protein, 213 aa, Streptomyces murayamaensis ATCC 21414
MDLADPVPAELAAKLASMGPAHEEVLADSVGLALLVVLETLDPAERMAFVLHDLFGLPYDEVAPIVGTGAEEACELADRARARVRRVDPLPDDGTTRLRRIVDAFLTASRGGDFATLTALLAPDVVLRADPEAVAVGAPTEVRGAGSVADAFSGRAKYAKLALVDGVVGAVWAPRGRPRVVFGFTVVDEKITGIDMRAAPERLDRLDLTILND